(19) United States
(12) Patent Application Publication
(10) Pub. No.: US 2024/0032541 A1
(43) Pub. Date: Feb. 1, 2024

(54) HERBICIDAL COMPOUNDS

(71) Applicant: SYNGENTA CROP PROTECTION AG, Basel (CH)

(72) Inventors: Jeffrey Steven WAILES, Bracknell, Berkshire (GB); Russell Colin VINER, Bracknell, Berkshire (GB); James Alan MORRIS, Bracknell, Berkshire (GB); Mary Bernadette ASPINALL, Bracknell, Berkshire (GB); Catherine Mary HOLDEN, Bracknell, Berkshire (GB); Philip Michael ELVES, Bracknell, Berkshire (GB); Sean NG, Bracknell, Berkshire (GB)

(73) Assignee: SYNGENTA CROP PROTECTION AG, Basel (CH)

(21) Appl. No.: 18/044,821

(22) PCT Filed: Sep. 6, 2021
(86) PCT No.: PCT/EP2021/074448

§ 371 Date: Mar. 10, 2023

(30) Foreign Application Priority Data
Sep. 11, 2020 (GB) 2014303.8

(51) Int. Cl. A01N 43/54; A01P 13/00; C07D 239/34; C07D 239/74; C07D 403/12; C07D 401/12; A01N 25/32

(52) U.S. Cl. CPC A01N 43/54; A01P 13/00; C07D 239/34; C07D 239/74; C07D 403/12; C07D 401/12; A01N 25/32

(57) ABSTRACT

The present invention relates to compounds of Formula (I), or an agronomically acceptable salt of said compounds wherein X¹, Y¹, Y², Z¹, Z₂, R¹, R², R⁶, R⁹ and n are as defined herein. The invention further relates to herbicidal compositions which comprise a compound of Formula (I) and to the use of compounds of Formula (I) for controlling weeds, in particular in crops of useful plants.

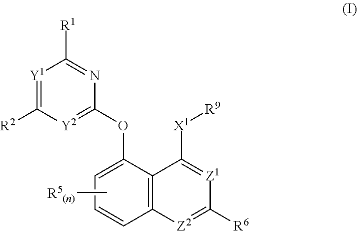

The present invention relates to novel herbicidal compounds, to processes for their preparation, to herbicidal compositions which comprise the novel compounds, and to their use for controlling weeds, in particular in crops of useful plants, or for inhibiting plant growth.

Thus, according to the present invention there is provided a compound of Formula (I):

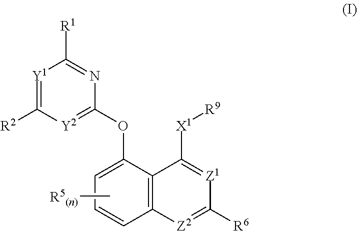

or an agronomically acceptable salt thereof,
Y¹ is N or CR³;
Y² is N or CR⁴;
with the proviso that Y¹ and Y² are not both N;
R¹ is selected from the group consisting of hydrogen, halogen, C₁-C₃alkyl and C₁-C₃haloalkyl;
R² is selected from the group consisting of hydrogen, halogen, C₁-C₃alkyl, C₁-C₃alkoxy-, C₁-C₃haloalkoxy- and C₁-C₃haloalkyl;
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
each R⁵ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁶ is independently selected from the group consisting of hydrogen, hydroxy, C₁-C₅alkyl, C₁-C₅haloalkyl-, C₁-C₅alkoxy-, C₁-C₆haloalkoxy- and C₃-C₆cycloalkyl-;
X¹ is CH₂ or O;
Z¹ is N or CR⁷;
Z² is N or CR⁸;
R⁷ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁸ is is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁹ is selected from the group consisting of C₁-C₅alkyl, C₁-C₅haloalkyl- and pyrimidin-2-yl wherein the pyrimidin-2-yl is optionally substituted by 1 or 2 substituents independently selected from the group consisting of halogen, CN, C₁-C₂ alkyl, C₁-C₂ alkoxy- and C₁-C₂ haloalkoxy-;
n=0, 1 or 2; and
p=0, 1 or 2.

C₁-C₄alkyl- and C₁-C₅alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₅haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl.

C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₂alkoxy includes, for example, methoxy and ethoxy.

C₁-C₅haloalkoxy- and C₁-C₄haloalkoxy- include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₄alkenyl- includes, for example, —CH═CH₂ (vinyl) and —CH₂—CH═CH₂ (allyl).

C₂-C₄alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention there is provided a compound of Formula (I) wherein Y¹ is CR³ and Y² is N (in this embodiment R¹ and R² are preferably hydrogen); or Y¹ is CR³ and R² is CR⁴ (in this embodiment R¹ and R² are preferably hydrogen); or Y¹ is N and Y² is CR⁴ (in this embodiment R¹ and R² are preferably hydrogen). In a more preferred embodiment of the present invention there is provided a compound of Formula (I) wherein Y¹ is CR³ and Y² is N wherein R³ is C₁-C₄alkyl (preferably methyl) or halo (preferably chloro), more preferably halo (preferably chloro).

In another embodiment of the present invention there is provided a compound of Formula (I) wherein Z¹ is CR⁷ (preferably CH) and Z² is CR³ (preferably CH); or Z¹ is CR⁷ (preferably CH) and Z² is N; or Z¹ is N and Z² is N; or Z¹ is N and Z² is CR^{B}. In a more preferred embodiment Z¹ is N and Z² is N.

In another embodiment of the present invention there is provided a compound of Formula (I) wherein n=0. In another embodiment of the present invention there is provided a compound of Formula (I) wherein n=1, wherein R⁵ is selected from the group consisting of fluoro, chloro, bromo and CN.

In another embodiment of the present invention there is provided a compound of Formula (I) wherein R⁶ is selected from the group consisting of hydrogen, C₁-C₆alkyl (preferably methyl), C₁-C₆alkoxy- (preferably methoxy) and C₁-C₆haloalkyl-(preferably CF₃). In another embodiment of the present invention there is provided a compound of Formula (I) wherein R⁶ is selected from the group consisting of hydrogen, C₁-C₆alkyl (preferably methyl) and C₁-C₆haloalkyl- (preferably CF₃).

In another embodiment of the present invention X¹ is CH₂.

In another embodiment of the present invention there is provided a compound of Formula (I) wherein R⁹ is pyrimidin-2-yl which is optionally substituted by 1 or 2 substituents independently selected from the group consisting of halogen, CN, C₁-C₂ alkyl, C₁-C₂ alkoxy- and C₁-C₂ haloalkoxy-, preferably chloro.

In another embodiment of the present invention there is provided a compound of Formula (I) wherein R⁹ is C₁-C₅alkyl or C₁-C₅haloalkyl-.

In preferred embodiment of the present invention X¹ is CH₂ and R⁹ is C₁-C₆alkyl or C₁-C₆haloalkyl-.

In a particularly preferred embodiment of the present invention R¹ and R² are hydrogen, Y¹ is CR³ and Y² is N wherein R³ is C₁-C₄alkyl (preferably methyl) or halo (preferably chloro), n is 0, R⁶ is selected from the group consisting of hydrogen, C₁-C₆alkyl (preferably methyl), C₁-C₆alkoxy- (preferably methoxy) and C₁-C₆haloalkyl-(preferably CF₃), Z¹ is N, Z² is N, X¹ is CH₂ and R⁹ is C₁-C₆alkyl or C₁-C₆haloalkyl-.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester,4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, tetrahydrofuran-2-ylmethyl (2R)-2-[(4-amino-3,5-dichloro-6-fluoro-2-pyridyl)oxy]propanoate, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate and 6-amino-5-chloro-2-(4-chloro-2-fluoro-3-methoxy-phenyl)pyrimidine-4-carboxylic acid.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012.

The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield@ summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

Processes for preparation of compounds, e.g. a compound of formula (I) (which optionally can be an agrochemically acceptable salt thereof), are now described, and form further aspects of the present invention.

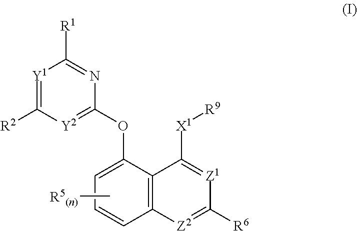

Processes for preparation of compounds, e.g. a compound of formula (I) (which optionally can be an agrochemically acceptable salt thereof), are now described, and form further aspects of the present invention.

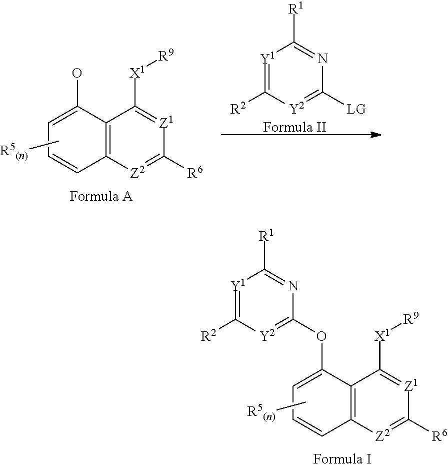

A compound of Formula I may be prepared from a compound of Formula A by reaction with a compound of Formula II (where LG represents a suitable leaving group such as F, Cl, Br or SO₂Me) in the presence of a suitable base and in a suitable solvent. Suitable bases may include NaH, K₂CO₃, Cs₂CO₃. Suitable solvents may include THF, CH₃CN or DMF. Compounds of Formula II are commercially available or may be prepared by known methods.

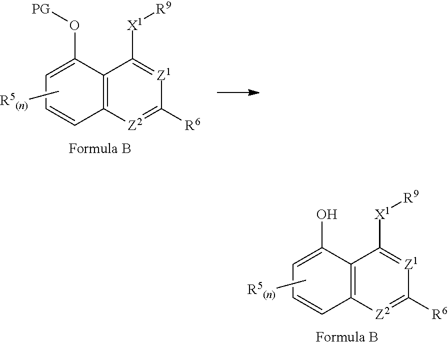

A compound of Formula A may be prepared from a compound of Formula B (where PG represents a suitable protecting group such as Me or Tf) by a deprotection reaction in a suitable solvent. Suitable deprotecting conditions may include BBr₃ or dodecanethiol/LiO^{t}Bu (for PG=Me) or K₂CO₃ (for PG=Tf. Suitable solvents may include DCM, DCE or CH₃CN.

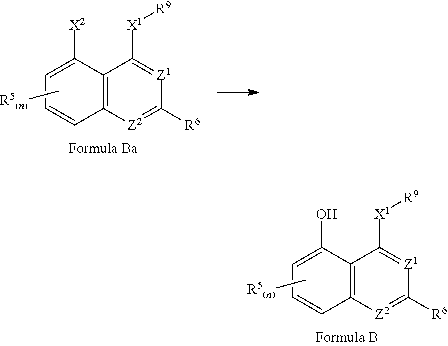

In an alternative method, a compound of Formula A may be prepared from compound of formula Ba where X2 is a leaving group like Cl, Br, F. It involves subjecting compound of formula Ba with nucleophilic surrogate for hydroxide such as acetohydroxamic acid in presence of a suitable base such as potassium carbonate and a suitable solvent such as dimethyl sulfoxide as found in literature Org letter 2016, 18, 2244-2247.

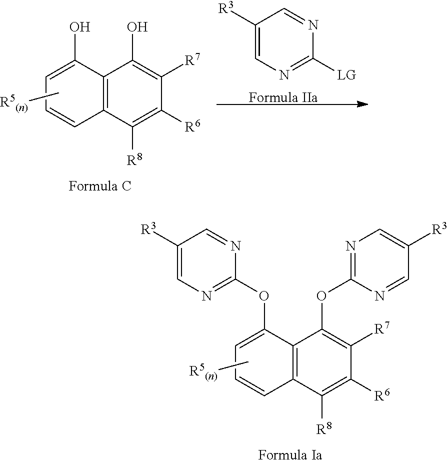

In an alternative method, a compound of Formula Ia (a compound of Formula I where X¹═O, Z¹═CR⁷, Z³═CR³ and R⁹=pyrimidin-2-yl) may be prepared from a compound of Formula C by reaction with a compound of formula Ia (a compound of Formula II where R¹═R²═H, Y¹═C—R³, Y²═N and LG represents a suitable leaving group such as F, Cl, Br or SO₂Me) in the presence of a suitable base and in a suitable solvent. Suitable bases may include K₂CO₃ or Cs₂CO₃. Suitable solvents may include CH₃CN or DMF. Compounds of Formula C and of Formula Ia are commercially available or may be prepared by known methods.

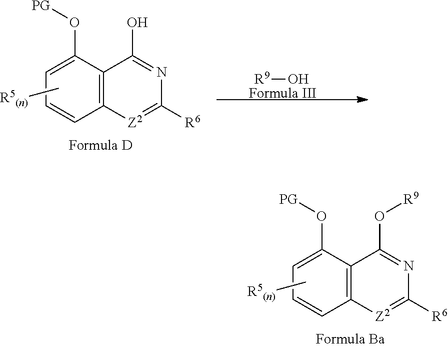

A compound of Formula Ba (a compound of Formula B where X¹═O and Z¹═N) may be prepared from a compound of Formula D by reaction with a compound of Formula III in the presence of a suitable phosphine and a suitable azodicarboxylate reagent in a suitable solvent. Suitable phosphines may include triphenylphosphine. Suitable azodicarboxylate reagents may include diisopropylazodicarboxylate. Suitable solvents may include chloroform. Compounds of Formula III are commercially available or may be prepared by known methods.

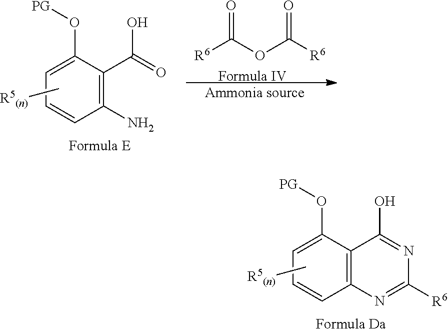

A compound of Formula Da (a compound of Formula D where Z²═N) may be prepared from a compound of Formula E by reaction with a compound of Formula IV and an ammonia source, in the presence of a suitable base and optionally in the presence of a suitable catalyst and in a suitable solvent. Suitable ammonia sources may include ammonium acetate. Suitable bases may include triethylamine. Suitable catalysts may include 4-dimethylaminopyridine. Suitable solvents may include toluene.

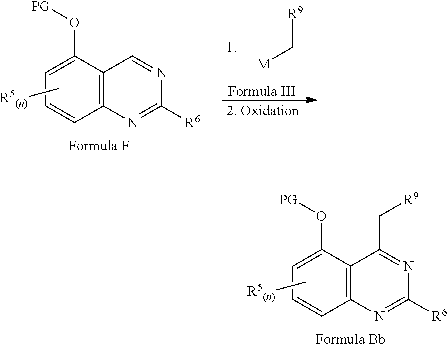

A compound of Formula Bb (a compound of Formula B where X¹═CH₂, Z¹ and Z²══N) may be prepared from a compound of Formula F in a two-step process. The first step involves reaction with a compound of Formula V (where M is a suitable organometallic such as Li or MgHal), optionally in the presence of a suitable catalyst and in a suitable solvent. Suitable catalysts may include lanthanum (III) chloride bis(lithium chloride) complex. Suitable solvents may include THF. The second step involves reaction with a suitable oxididising agent in a suitable solvent. Suitable oxidising agents may include 3-dichloro-5,6-dicyano-1,4-benzoquinone or potassium ferricyanide. Suitable solvents may include THF or Et₂O/water.

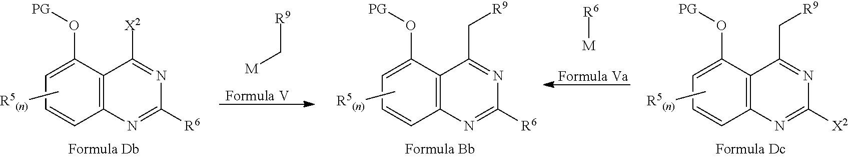

Alternatively compound of formula Bb can also be prepared from compound of formula Db or Dc where X² is either Cl, Br or F. It involves reaction with compound of formula V or Va with compound of formula Db or Dc (where M is a suitable organometallic such as Li or MgHal), optionally in the presence of a suitable catalyst and in a suitable solvent. Suitable catalyst may include copper chloride, iron(III) acetylacetonate and suitable solvent may include tetrahydrofuran.

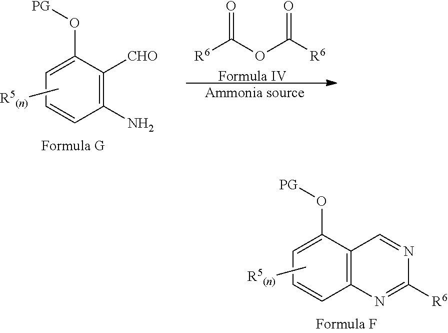

A compound of Formula F may be prepared from a compound of Formula G by reaction with a compound of Formula IV and an ammonia source, in the presence of a suitable base and optionally in the presence of a suitable catalyst and in a suitable solvent. Suitable ammonia sources may include ammonium acetate. Suitable bases may include triethylamine. Suitable catalysts may include 4-dimethylaminopyridine. Suitable solvents may include toluene. Compounds of Formula G and of Formula IV are commercially available or may be prepared by known methods.

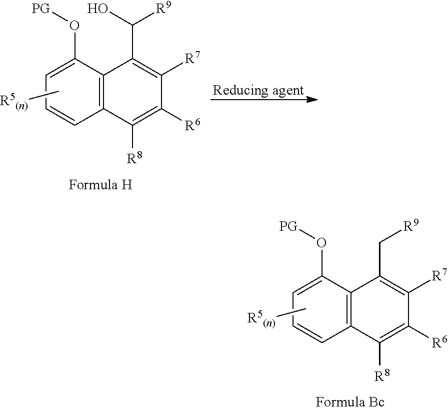

A compound of Formula Bc (a compound of Formula B where X¹═CH₂, Z¹═CR⁷, Z² ═CR⁸) may be prepared from a compound of formula H by reaction with a suitable reducing agent in a suitable solvent. Suitable reducing agents may include triethylsilane/trifluoroacetic acid. Suitable solvents may include DCM.

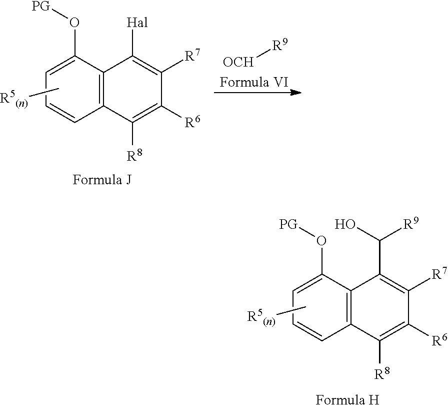

A compound of Formula H may be prepared from a compound of Formula J by metalation with a suitable organometallic reagent and reaction with a compound of Formula VI in a suitable solvent. Suitable organometallic reagents may include n-butyl lithium. Suitable solvents may include THF. Compounds of Formula VI and of Formula J are commercially available or may be prepared by known methods.

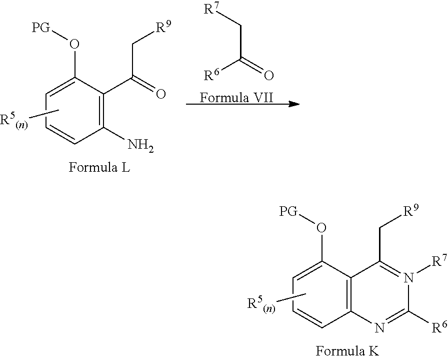

Compounds of Formula K may be prepared from a compound of Formula L by reacting it with compound of formula VII in presence of a suitable catalyst such as p-toluene sulfonic acid and a suitable solvent such as dimethyl formamide. Compounds of formula L may be prepared by known methods.

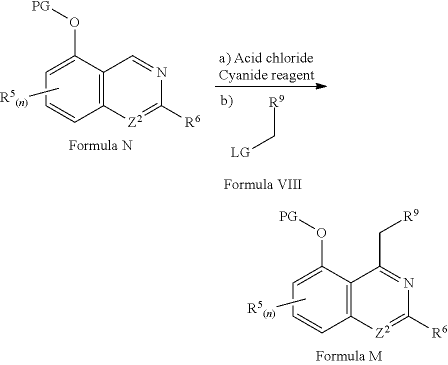

Compounds of formula M may be prepared from a compound of formula N by reacting it with a acid chloride and cyanide reagent followed by reacting it with compound of formula VIII in presence of suitable base such as sodium hydride.

Suitable cyanide reagent may be trimethylsilyl cyanide. Compounds of formula N are commercially available or may be prepared by known methods.

The following non-limiting examples provide specific synthesis methods for representative compounds of the present invention, as referred to in the Table below.

LCMS spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### EXAMPLE 1: SYNTHESIS OF 5-CHLORO-2-[[8-(5-CHLOROPYRIMIDIN-2-YL)OXY-1-NAPHTHYL]OXY]PYRIMIDINE (COMPOUND 1.001)

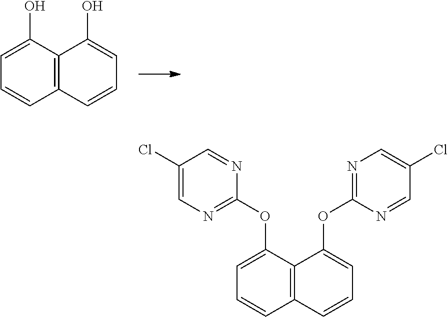 [SMILES: Oc1cccc2cccc(O)c12 ; Clc1cnc(Oc2cccc3cccc(Oc4ncc(Cl)cn4)c23)nc1]

To a stirred solution of naphthalene-1,8-diol (0.200 g, 1.25 mmol) and K₂CO₃ (0.88 g, 6.25 mmol) in N,N-dimethylformamide (6.2 mL) was added 2,5-dichloropyrimidine (0.558 g, 3.75 mmol) and the resultant mixture was heated to 80° C. for 1 h. The reaction was allowed to cool to RT, diluted with H₂O and extracted with DCM. The combined organic extracts were dried over MgSO₄ and evaporated to dryness under reduced pressure. The crude product was purified by flash chromatography on silica gel using a gradient of 0-30% EtOAc in cyclohexane as eluent to give the desired product (0.204 g, 42%) as a white solid.

¹H NMR (400 MHz, CDCl₃) δ 8.41 (s, 4H), 7.89 (dd, 2H), 7.55 (t, 2H), 7.21 (dd, 2H)

### EXAMPLE 2: SYNTHESIS OF 5-CHLORO-2-[[8-(4,4,4-TRIFLUOROBUTYL)-1-NAPHTHYL]OXY]PYRIMIDINE (COMPOUND 1.002)

### Step 1: Synthesis of 4,4,4-trifluoro-1-(8-methoxy-1-naphthyl)butan-1-ol

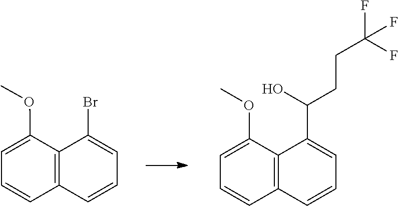 [SMILES: COc1cccc2cccc(Br)c12 ; COc1cccc2cccc(C(O)CCC(F)(F)F)c12]

To a solution of 1-bromo-8-methoxy-naphthalene (0.23 g, 0.97 mmol) in tetrahydrofuran (9.7 mL) at −78° C. under an N₂ atmosphere was added dropwise n-butyllithium (0.47 mL of a 2.5M solution in hexanes, 1.2 mmol). The mixture was stirred at −78° C. for 30 min then 4,4,4-trifluorobutanal (0.13 g, 1.1 mmol) in 1 mL THF was added and the reaction was stirred for 1 h at −78° C. The reaction was allowed to warm to room temp and quenched by the addition of saturated aqueous ammonium chloride solution, extracted into ethyl acetate, dried over MgSO₄ and evaporated to dryness under reduced pressure. The crude product was purified by flash chromatography on silica gel using a gradient of 0-30% EtOAc/cyclohexane to give the desired product (92 mg, 33%).

¹H NMR (400 MHz, CDCl₃) δ 7.75-7.70 (m, 2H), 7.48-7.35 (m, 3H), 6.90 (d, 1H), 5.80 (br, 1H), 3.97 (s, 3H), 2.75 (br, 1H), 2.50-2.25 (m, 2H), 2.22-2.15 (m, 1H), 2.00-1.92 (m, 1H).

### Step 2: Synthesis of 1-methoxy-8-(4,4,4-trifluorobutyl)naphthalene

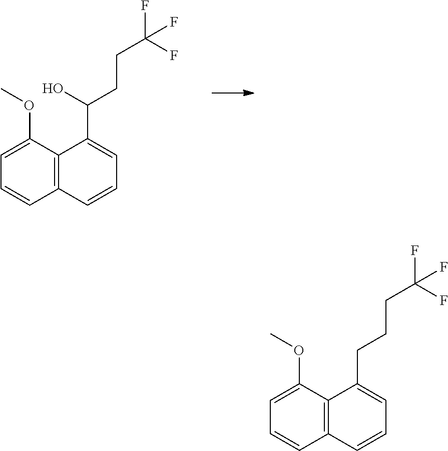 [SMILES: COc1cccc2cccc(C(O)CCC(F)(F)F)c12 ; COc1cccc2cccc(CCCC(F)(F)F)c12]

To a stirred solution of 4,4,4-trifluoro-1-(8-methoxy-1-naphthyl)butan-1-ol (0.080 g, 0.28 mmol) in dichloromethane (0.95 mL) was added triethylsilane (0.18 mL, 1.1 mmol). After stirring for 15 minutes, trifluoroacetic acid (0.13 mL, 1.7 mmol) was added and the reaction was heated to 40° C. overnight. The reaction was allowed to cool to room temperature and evaporated to dryness under reduced pressure. The crude product was purified by flash chromatography using a gradient of 0-20% EtOAc/cyclohexane as eluent to give the desired product (19 mg, 25%).

¹H NMR (400 MHz, CDCl₃) δ 7.67 (dd, 1H), 7.45-7.40 (m, 1H), 7.40-7.32 (m, 2H), 7.19 (d, 1H), 6.88-6.82 (m, 1H), 3.96 (s, 3H), 3.36-3.28 (m, 2H), 2.23-2.10 (m, 2H), 2.00-1.90 (m, 2H)

### Step 3: Synthesis of 8-(4,4,4-trifluorobutyl)naphthalen-1-ol

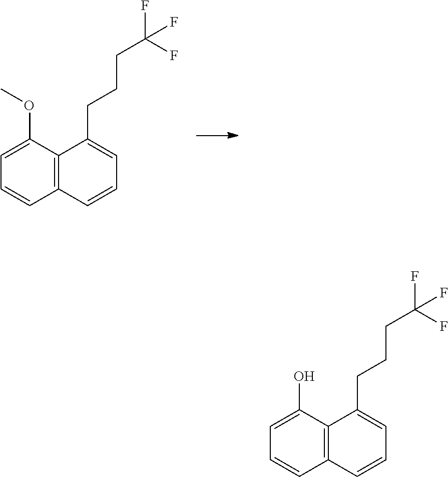 [SMILES: COc1cccc2cccc(CCCC(F)(F)F)c12 ; Oc1cccc2cccc(CCCC(F)(F)F)c12]

To a stirred solution of 1-methoxy-8-(4,4,4-trifluorobutyl)naphthalene (0.03 g, 0.112 mmol) in DCM (1.12 mL) at 0° C. under an atmosphere of N₂ was added dropwise boron tribromide (0.28 mL of a 1M solution in DCM, 0.28 mmol). The reaction was stirred at room temperature for 6 h, then quenched by the addition of sat. aq. NaHCO₃. The organic phase was separated and evaporated to dryness under reduced pressure. The crude product was purified by flash chromatography on silica gel using a gradient of 0-30% EtOAc/cyclohexane as eluent to give the desired product (12 mg, 42%).

¹H NMR (400 MHz, CDCl₃) δ 7.72-7.61 (m, 1H), 7.43 (dd, 1H), 7.38-7.31 (m, 1H), 7.29-7.22 (m, 1H), 7.22-7.14 (m, 1H), 6.74-6.68 (m, 1H), 5.33-5.23 (m, 1H), 3.37-3.26 (m, 2H), 2.23-2.08 (m, 2H), 2.06-1.94 (m, 2H).

### Step 4: Synthesis of 5-chloro-2-[[8-(4,4,4-trifluorobutyl)-1-naphthyl]oxy]pyrimidine (compound 1.002)

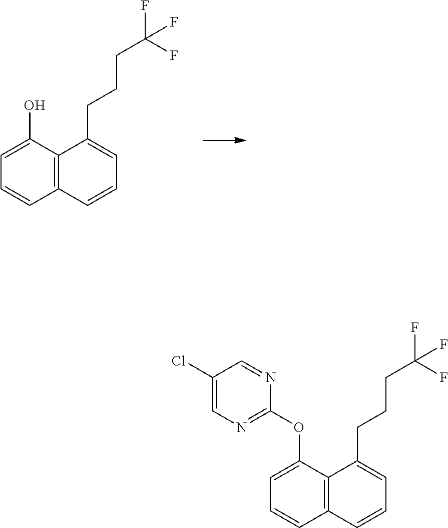 [SMILES: Oc1cccc2cccc(CCCC(F)(F)F)c12 ; FC(F)(F)CCCc1cccc2cccc(Oc3ncc(Cl)cn3)c12]

To a stirred solution of 8-(4,4,4-trifluorobutyl)naphthalen-1-ol (12 mg, 0.047 mmol) and potassium carbonate (13 mg, 0.094 mmol) in DMF (0.12 mL) was added 2,5-dichloropyrimidine (8.5 mg, 0.057 mmol). The reaction mixture was heated at 80° C. for 3.5 h, then allowed to cool to RT. The reaction was diluted with water and extracted with EtOAc (×2). The combined organic extracts were dried over MgSO₄ and evaporated to dryness under reduced pressure. The crude product was purified by flash chromatography on silica gel using a gradient of 0-20% EtOAc/cyclohexane as eluent to give the desired product (16 mg, 94%).

¹H NMR (400 MHz, CDCl₃) δ 8.50 (s, 2H), 7.85-7.78 (m, 2H), 7.48 (t, 1H), 7.40 (t, 1H), 7.30 (d, 1H), 7.20 (d, 1H), 3.10 (t, 2H), 2.20-2.07 (m, 2H), 1.92-1.83 (m, 2H)

### EXAMPLE 3: SYNTHESIS OF 5-(5-CHLOROPYRIMIDIN-2-YL)OXY-4-(4,4,4-TRIFLUOROBUTYL)QUINAZOLINE (COMPOUND 1.003)

### Step 1: Synthesis of 5-methoxy-4-(4,4,4-trifluorobutyl)quinazoline

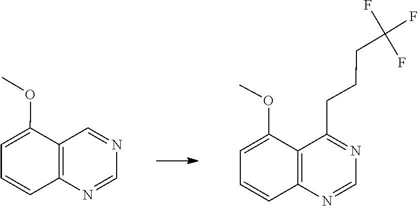 [SMILES: COc1cccc2ncncc12 ; COc1cccc2ncnc(CCCC(F)(F)F)c12]

To stirred magnesium turnings (0.155 g, 6.24 mmol) in THF (60 mL) was added a crystal of iodine. The reaction was stirred for 10 mins and a small amount of 1-bromo-4,4,4-trifluorobutane was added. The reaction was heated with a heat gun until the solution went from yellow to colourless. The remaining 1-bromo-4,4,4-trifluorobutane (1.20 g, 6.24 mmol) was added dropwise and then the reaction heated at 75° C. until the no solid remained (˜30 minutes) then allowed to cool to RT.

A solution of 5-methoxyquinazoline (0.500 g, 3.12 mmol) in THF (30 mL) was added to the above formed bromo(4,4,4-trifluorobutyl)magnesium and the reaction was stirred at RT for 1 hour. The reaction was quenched with saturated aqueous ammonium chloride and extracted with EtOAc. The combined organics were washed with brine, dried over MgSO₄ and evaporated to dryness under reduced pressure to give 5-methoxy-4-(4,4,4-trifluorobutyl)-3,4-dihydroquinazoline as a pale orange gum.

The crude intermediate was dissolved in diethyl ether (15 mL) and KOH (2.5 mL of a 20% aqueous solution) and potassium ferricyanide (2.08 g, 6.24 mmol) added and stirred vigorously at RT for 44 hours. The reaction was diluted with water and extracted with diethyl ether. The combined organic extracts were dried over MgSO₄ and evaporated to dryness under reduced pressure. The crude product was purified by flash chromatography on silica gel using a gradient of 0-50% EtOAc/cyclohexane as eluent to give the desired product (0.548 g, 65%) as a white solid.

¹H NMR (400 MHz, CDCl₃) δ 9.12 (s, 1H), 7.82-7.75 (m, 1H), 7.62 (dd, 1H), 6.98 (d, 1H), 4.03 (s, 3H), 3.58-3.49 (m, 2H), 2.37-2.20 (m, 2H) 2.16-2.05 (m, 2H).

### Step 2: Synthesis of 5-(5-chloropyrimidin-2-yl)oxy-4-(4,4,4-trifluorobutyl)quinazoline (compound 1.003)

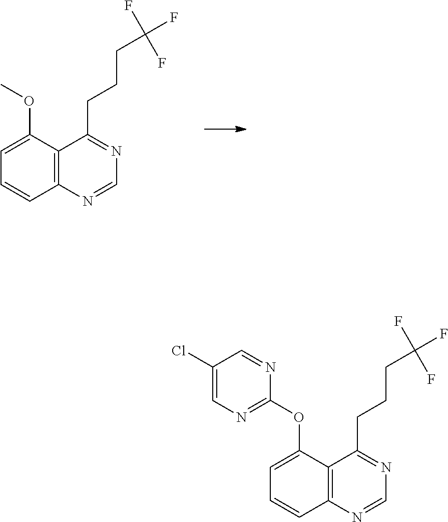 [SMILES: COc1cccc2ncnc(CCCC(F)(F)F)c12 ; FC(F)(F)CCCc1ncnc2cccc(Oc3ncc(Cl)cn3)c12]

To a stirred solution of 5-methoxy-4-(4,4,4-trifluorobutyl)quinazoline (0.050 g, 0.19 mmol) and 1-dodecanethiol (0.076 g, 0.37 mmol) in DMF (0.5 mL) was added LiO^{t}Bu (0.37 mL of a 1M solution in THF, 0.37 mmol). The reaction was heated at 100° C. for 1.5 hours then allowed to cool to RT.

To the cooled reaction mixture were added Cs₂CO₃ (0.15 g, 0.46 mmol) and 2,5-dichloropyrimidine (0.073 g, 0.46 mmol) and the reaction heated to 80° C. for 18 hours. The reaction was allowed to cool to RT and evaporated to dryness under reduced pressure. The crude product was purified by flash chromatography on silica gel using a gradient of 0-50% EtOAc/cyclohexane as eluent to give the desired product (0.032 g, 47%) as a pale yellow solid.

¹H NMR (400 MHz, CDCl₃) δ 9.22 (s, 1H), 8.53 (s, 2H), 8.02 (dd, 1H), 7.90 (t, 1H), 7.37 (dd, 1H), 3.38 (t, 2H), 2.27-2.18 (m, 2H), 2.08-2.04 (m, 2H)

### EXAMPLE 4: SYNTHESIS OF 5-(5-CHLOROPYRIMIDIN-2-YL)OXY-4-(4,4,4-TRIFLUOROBUTYL)-2-(TRIFLUOROMETHYL)QUINAZOLINE (1.004)

### Step 1: Synthesis of 5-methoxy-2-(trifluoromethyl)quinazoline

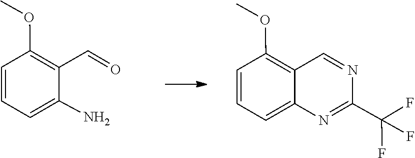 [SMILES: COc1cccc(N)c1C=O ; COc1cccc2nc(C(F)(F)F)ncc12]

To a stirred solution of 2-amino-6-methoxy-benzaldehyde (1.00 g, 6.62 mmol) and 4-dimethylaminopyridine (0.082 g, 0.662 mmol) in toluene (20 mL) at 0° C. was added triethylamine (2.04 mL, 14.6 mmol) followed by the slow addition of trifluoroacetic acid anhyride (1.43 mL, 9.92 mmol). The reaction was allowed to warm to RT and stirred for 18 hours. To the reaction was added ammonium acetate (1.16 g, 14.6 mmol), the reaction heated at 90° C. for 24 hours then allowed to cool to RT. The reaction was diluted with water, stirred vigorously for 30 minutes, then the phases separated and the aqueous phase extracted with EtOAc (×2). The combined organic phases were dried over MgSO₄ and evaporated to dryness under reduced pressure. The crude product was purified by flash chromatography on silica gel using a gradient of 0-50% EtOAc/cyclohexane as eluent to give the desired product (0.69 g, 46%) as a pale yellow solid.

¹H NMR (400 MHz, CDCl₃) δ 9.87 (s, 1H), 7.95 (t, 1H), 7.75 (d, 1H), 7.08 (d, 1H), 4.09 (s, 3H)

### Step 2: Synthesis of 5-methoxy-4-(4,4,4-trifluorobutyl)-2-(trifluoromethyl)quinazoline

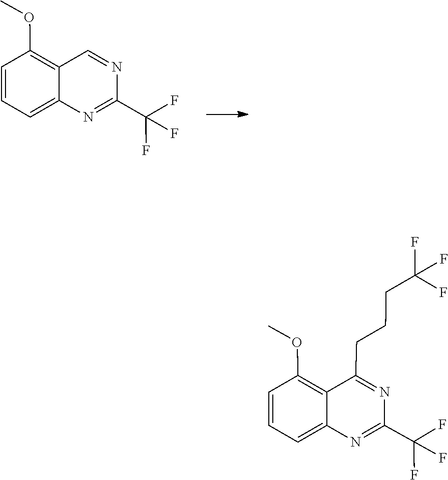 [SMILES: COc1cccc2nc(C(F)(F)F)ncc12 ; COc1cccc2nc(C(F)(F)F)nc(CCCC(F)(F)F)c12]

To stirred magnesium turnings (0.170 g, 6.84 mmol) in THF (10 mL) at RT under an N₂ atmosphere was added a crystal of iodine. The reaction was stirred for 10 mins and a small amount of 1-bromo-4,4,4-trifluorobutane was added. The reaction was heated with a heat gun until the solution went from yellow to colourless. The remaining 1-bromo-4,4,4-trifluorobutane (0.848 mL, 6.84 mmol) was added dropwise and then heated at 75° C. for 30 minutes until no solid remained.

To the Grignard reagent formed above was added 5-methoxy-2-(trifluoromethyl)quinazoline (0.52 g, 2.28 mmol) and lanthanum(III) chloride bis(lithium chloride) complex (11.4 mL of a 0.6M solution in THF, 6.84 mmol) and the reaction was stirred at RT for 18 hours. The reaction was quenched with saturated aqueous ammonium chloride solution and extracted with EtOAc (×2). The combined organic extracts were washed with brine, dried over MgSO₄ and evaporated to dryness under reduced pressure to give 5-methoxy-4-(4,4,4-trifluorobutyl)-2-(trifluoromethyl)-3,4-dihydroquinazoline as a pale orange gum.

To a solution of the crude 5-methoxy-4-(4,4,4-trifluorobutyl)-2-(trifluoromethyl)-3,4-dihydroquinazoline in THF (10 mL) at 0° C. was added 2,3-dichloro-5,6-dicyano-1,4-benzoquinone (0.581 g, 2.51 mmol). The reaction was allowed to warm to RT and stirred for 2 hours. The reaction was cooled to 0° C., quenched with 2M NaOH and stirred for 5 minutes then extracted with EtOAc (×2). The combined organic extracts were washed with water and brine, dried over MgSO₄ and evaporated to dryness under reduced pressure. The crude product was purified by flash chromatography on silica gel using a gradient of 0-10% EtOAc/cyclohexane as eluent to give the desired product (0.377 g, 49%) as a pale yellow solid.

¹H NMR (400 MHz, CDCl₃) δ 7.93-7.84 (m, 1H), 7.76 (dd, 1H), 7.10 (d, 1H), 4.06 (s, 3H), 3.66-3.55 (m, 2H), 2.39-2.24 (m, 2H), 2.29-2.08 (m, 2H).

### Step 3: Synthesis of 4-(4,4,4-trifluorobutyl)-2-(trifluoromethyl)quinazolin-5-ol

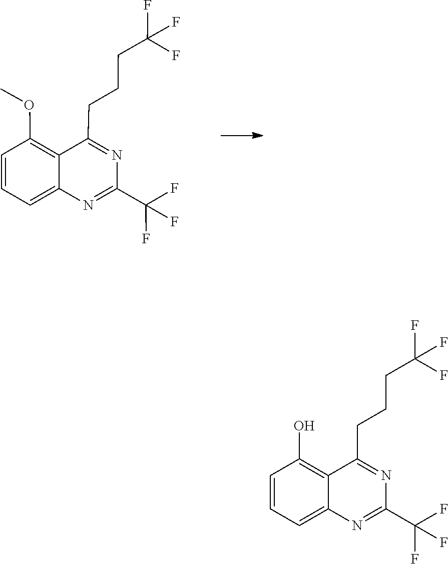 [SMILES: COc1cccc2nc(C(F)(F)F)nc(CCCC(F)(F)F)c12 ; Oc1cccc2nc(C(F)(F)F)nc(CCCC(F)(F)F)c12]

To a solution of 5-methoxy-4-(4,4,4-trifluorobutyl)-2-(trifluoromethyl)quinazoline (0.360 g, 1.06 mmol) and 1-dodecanethiol (0.52 mL, 2.13 mmol) in DMF (3.6 mL) was added lithium t-butoxide (2.13 mL of a 1M solution in THF, 2.13 mmol). The reaction was heated at 100° C. for 1.5 hours, then allowed to cool to RT, quenched with 1M HCl and extracted with EtOAc (×2). The combined organic extracts were washed with brine, dried over MgSO₄ and evaporated to dryness under reduced pressure. The crude product was purified by flash chromatography on silica gel using a gradient of 0-20% EtOAc/cyclohexane as eluent to give the desired product (0.250 g, 72%) as a pale yellow solid.

¹H NMR (400 MHz, CDCl₃) δ 7.83-7.71 (m, 2H), 7.05 (dd, 1H), 6.54 (br s, 1H), 3.66 (t, 2H), 2.39-2.24 (m, 2H), 2.24-2.14 (m, 2H).

### Step 4: Synthesis of 5-(5-chloropyrimidin-2-yl)oxy-4-(4,4,4-trifluorobutyl)-2-(trifluoromethyl)quinazoline (1.004)

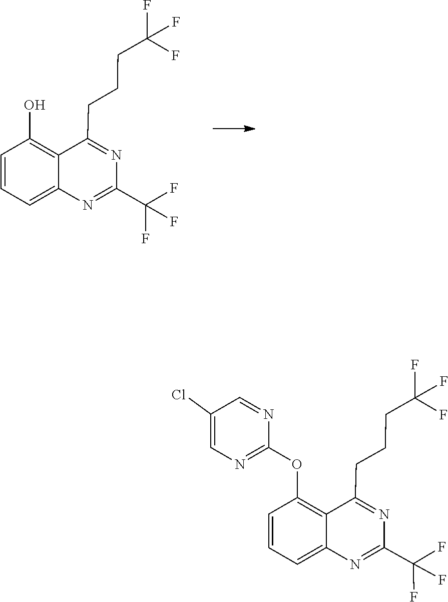 [SMILES: Oc1cccc2nc(C(F)(F)F)nc(CCCC(F)(F)F)c12 ; FC(F)(F)CCCc1nc(C(F)(F)F)nc2cccc(Oc3ncc(Cl)cn3)c12]

To a stirred suspension of sodium hydride (0.019 g of a 60% suspension in mineral oil, 0.463 mmol) in THF (2 mL) at 0° C. under an N₂ atmosphere was added dropwise a solution of 4-(4,4,4-trifluorobutyl)-2-(trifluoromethyl)quinazolin-5-ol (0.100 g, 0.308 mmol) in THF (2 mL). The reaction was stirred at 0° C. for 30 minutes then 2,5-dichloropyrimidine (0.073 g, 0.463 mmol) in THF (2 mL) was added dropwise. The reaction was heated at reflux for 96 hours, then allowed to cool to RT and evaporated to dryness under reduced pressure. The crude product was purified by flash chromatography on silica gel using a gradient of 0-10% EtOAc/cyclohexane as eluent to give the desired product (0.057 g, 42%) as a pale yellow solid.

¹H NMR (400 MHz, CDCl₃) δ 8.54 (s, 2H), 8.16 (d, 1H), 8.02 (t, 1H), 7.51 (d, 1H), 3.47 (t, 2H), 2.28-2.21 (m, 2H), 2.12-2.09 (m, 2H)

### EXAMPLE 5: SYNTHESIS OF 5-(5-CHLOROPYRIMIDIN-2-YL)OXY-2-(TRIFLUOROMETHYL)-4-(3,3,3-TRIFLUOROPROPOXY)QUINAZOLINE (1.006)

### Step 1: Synthesis of 5-methoxy-2-(trifluoromethyl)-3H-quinazolin-4-one

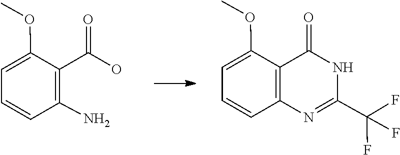

To a stirred mixture of 2-amino-6-methoxy-benzoic acid (2.00 g, 12.0 mmol) and 4-dimethylaminopyridine (0.148 g, 1.20 mmol) in toluene (36 mL) at 0° C. was added triethylamine (7.39 mL, 52.6 mmol) followed by dropwise addition of trifluoroacetic acid anhydride (5.35 mL, 37.1 mmol). The reaction mixture was allowed to warm to RT and stirred overnight, then ammonium acetate (4.18 g, 52.6 mmol) was added and reaction was heated at 90° C. overnight. The reaction was allowed to cool to RT, diluted with water (50 mL) and stirred vigorously. The resultant solid was filtered, washed with water and Et₂O and vacuum dried to give the desired product (1.57 g, 54%) as a cream solid.

¹H NMR (400 MHz, d₆-DMSO) δ 13.29 (br s, 1H), 7.80 (t, 1H), 7.31 (d, 1H), 7.20 (d, 1H), 3.90 (s, 3H).

### Step 2: Synthesis of 5-hydroxy-2-(trifluoromethyl)-3H-quinazolin-4-one

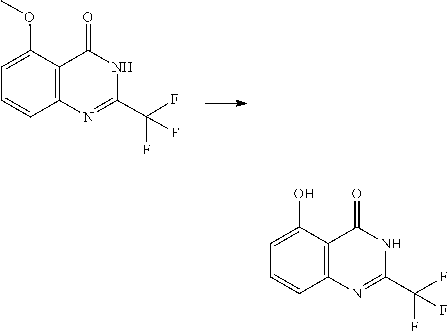 [SMILES: COc1cccc2nc(C(F)(F)F)[nH]c(=O)c12 ; O=c1[nH]c(C(F)(F)F)nc2cccc(O)c12]

To a solution of 5-methoxy-2-(trifluoromethyl)-3H-quinazolin-4-one (0.500 g, 2.05 mmol) in DCE (41.0 mL) at 0° C. was added dropwise boron tribromide (5.12 mL of a 1M solution in DCM, 5.12 mmol). The reaction was allowed to warm to RT and then heated at reflux overnight. The reaction was allowed to cool to RT, quenched with water, made basic with saturated aqueous NaHCO₃ solution and extracted with EtOAc (×3). The combined organic extracts were washed with water and brine, dried over MgSO₄ and evaporated to dryness under reduced pressure to give the desired product (0.445 g, 94%) as a brown solid.

¹H NMR (400 MHz, d6-DMSO) δ 11.74 (br, 1H), 7.77 (t, 1H), 7.27 (d, 1H), 7.02 (d, 1H)

### Step 3: Synthesis of [4-oxo-2-(trifluoromethyl)-3H-quinazolin-5-yl]trifluoromethanesulfonate

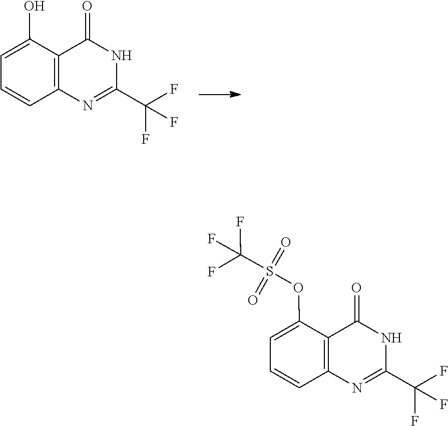 [SMILES: O=c1[nH]c(C(F)(F)F)nc2cccc(O)c12 ; O=c1[nH]c(C(F)(F)F)nc2cccc(OS(=O)(=O)C(F)(F)F)c12]

To a suspension of 5-hydroxy-2-(trifluoromethyl)-3H-quinazolin-4-one (0.435 g, 1.89 mmol) in THF (8.70 mL) at 0° C. was added Cs₂CO₃ (0.616 g, 1.89 mmol), followed by 1,1,1-trifluoro-N-(2-pyridyl)-N-(trifluoromethylsulfonyl)methanesulfonamide (0.677 g, 1.89 mmol). After 1 hour the reaction was allowed to warm to RT and stirred for 72 h. The reaction was cooled to 0° C. and further Cs₂CO₃ (300 mg) and 1,1,1-trifluoro-N-(2-pyridyl)-N-(trifluoromethylsulfonyl)methanesulfonamide (330 mg) were added and stirred overnight at RT. The reaction was heated at 40° C. for 1 hour and then heated at 50° C. overnight. The reaction was allowed to cool to RT, then quenched with water and extracted with EtOAc (×3). The combined organic extracts were washed with brine, dried over MgSO₄ and evaporated to dryness under reduced pressure. The crude product was purified by flash chromatography on silica gel using a gradient of 0-100% EtOAc/cyclohexane as eluent to give the desired product (0.109 g) as a beige solid as an inseparable mixture with starting material.

### Step 4: Synthesis of [2-(trifluoromethyl)-4-(3,3,3-trifluoropropoxy)quinazolin-5-yl] trifluoromethanesulfonate

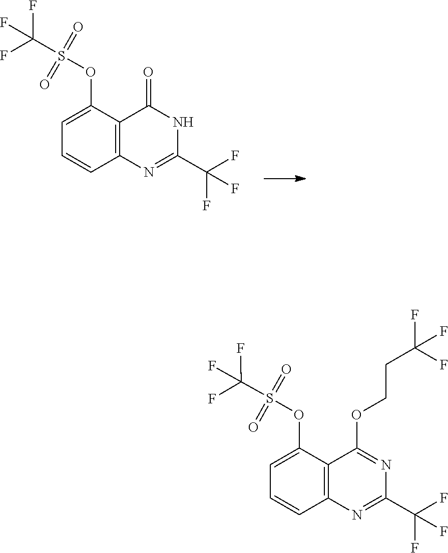 [SMILES: O=c1[nH]c(C(F)(F)F)nc2cccc(OS(=O)(=O)C(F)(F)F)c12 ; O=S(=O)(Oc1cccc2nc(C(F)(F)F)nc(OCCC(F)(F)F)c12)C(F)(F)F]

To a suspension of [4-oxo-2-(trifluoromethyl)-3H-quinazolin-5-yl]trifluoromethanesulfonate (0.060 g, 0.17 mmol) and triphenylphosphine (0.11 g, 0.41 mmol) in chloroform (1.2 mL) at RT under an N₂ atmosphere was added 3,3,3-trifluoropropan-1-ol (0.037 mL, 0.41 mmol). The solution was cooled to 0° C. and DIAD (0.82 mL, 0.41 mmol) was added dropwise. The reaction mixture was stirred at 0° for 10 minutes and then allowed to warm to RT. The reaction was quenched with water and extracted with EtOAc (×3). The combined organic extracts were washed with 2M HCl then brine, dried over MgSO₄ and evaporated to dryness under reduced pressure. The crude product was purified by flash chromatography on silica gel using a gradient of 0-40% EtOAc/cyclohexane as eluent to give the desired product (0.068 g, 90%) as a colourless gum.

¹H NMR (400 MHz, CDCl₃) δ 8.19 (d, 1H), 7.99 (t, 1H), 7.59 (d, 1H), 4.87 (t, 2H), 2.89 (m, 2H)

### Step 5: Synthesis of Synthesis of 5-(5-chloropyrimidin-2-yl)oxy-2-(trifluoromethyl)-4-(3,3,3-trifluoropropoxy)quinazoline (1.006)

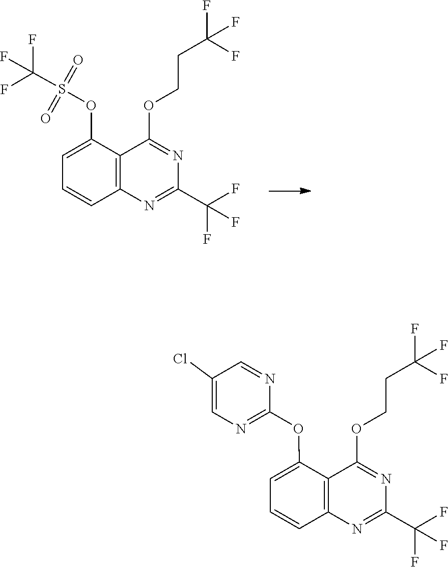 [SMILES: O=S(=O)(Oc1cccc2nc(C(F)(F)F)nc(OCCC(F)(F)F)c12)C(F)(F)F ; FC(F)(F)CCOc1nc(C(F)(F)F)nc2cccc(Oc3ncc(Cl)cn3)c12]

To a solution of [2-(trifluoromethyl)-4-(3,3,3-trifluoropropoxy)quinazolin-5-yl]trifluoromethanesulfonate (0.068 g, 0.15 mmol) in acetonitrile (1.1 mL) was added Cs₂CO₃ (0.21 g, 1.1 mmol) and 2,5-dichloropyrimidine (0.033 g, 0.22 mmol) and the reaction heated at 80° C. overnight. The reaction was diluted with water and extracted with EtOAc (×3). The combined organic extracts were washed with brine, dried over MgSO₄ and evaporated to dryness under reduced pressure. The crude product was purified by flash chromatography on silica gel using a gradient of 0-40% EtOAc/cyclohexane as eluent to give the desired product (24 mg, 37%) as a white solid.

¹H NMR (400 MHz, CDCl₃) δ 8.47 (s, 2H), 8.08 (d. 1H), 8.00 (t, 1H), 7.48 (d, 1H), 4.67 (t, 2H), 2.48 (m, 2H)

### EXAMPLE 6: SYNTHESIS OF 5-(5-CHLOROPYRIMIDIN-2-YL)OXY-2-METHYL-4-(4,4,4-TRIFLUOROBUTYL)QUINAZOLINE (1.013)

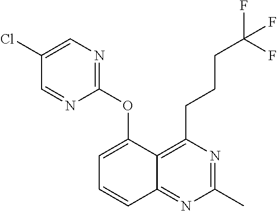 [SMILES: Cc1nc(CCCC(F)(F)F)c2c(Oc3ncc(Cl)cn3)cccc2n1]

### Step 1: Synthesis of 1-(2,6-difluorophenyl)-5,5,5-trifluoro-pentan-1-ol

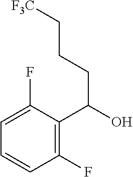 [SMILES: OC(CCCC(F)(F)F)c1c(F)cccc1F]

To stirred magnesium turnings (3.42 g, 140.74 mmol) in tetrahydrofuran (40 mL) was added a crystal of iodine. The reaction was stirred for 10 mins and heated to reflux. Then added a small amount of 1-bromo-4,4,4-trifluorobutane in tetrahydrofuran. The heating was continued until the solution went from yellow to grey in colour. The remaining 1-bromo-4,4,4-trifluorobutane (20.16 g, 105.55 mmol) solution in tetrahydrofuran (70 ml) was added dropwise under heating condition and then the reaction heated at 75° C. for 1 h, then allowed to cool to RT. The above solution of bromo(4,4,4-trifluorobutyl)magnesium in tetrahydrofuran was added dropwise to a cooled solution of 2,6-difluorobenzaldehyde (10 g, 70.37 mmol) in tetrahydrofuran (80 mL) at 0° C. and the reaction was stirred at RT for 16 hour. The reaction was quenched with saturated aqueous ammonium chloride and extracted with ethyl acetate (3×500 mL). The combined organics were washed with brine, dried over sodium sulfate and evaporated to dryness under reduced pressure to give 1-(2,6-difluorophenyl)-5,5,5-trifluoro-pentan-1-ol (17.8 g, 99.5%) as yellow oil.

¹H NMR (400 MHz, CHLOROFORM-d) δ ppm 7.15-7.23 (m, 1H), 6.80-6.90 (m, 2H), 5.01 (br s, 1H), 2.11-2.30 (m, 2H), 1.97-2.15 (m, 2H) 1.69-1.90 (m, 2H)

### Step 2: Synthesis of 1-(2,6-difluorophenyl)-5,5,5-trifluoro-pentan-1-one

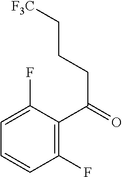 [SMILES: O=C(CCCC(F)(F)F)c1c(F)cccc1F]

To a solution of 1-(2,6-difluorophenyl)-5,5,5-trifluoro-pentan-1-ol (17 g, 66.87 mmol) in acetonitrile (340 mL) added (1,1-diacetoxy-3-oxo-1lambda5,2-benziodoxol-1-yl) acetate (30.70 g, 70.220 mmol) portion wise at 0° C. and warmed the reaction mass to rt, stirred at room temperature for 2 h. The reaction was quenched with saturated solution of sodium thiosulfate (32.04 g, 200.63 mmol) in water (1000 mL), followed by sodium bicarbonate solution and extracted in ethyl acetate (3×500 mL), dried over anhydrous sodium sulfate, filtered and concentrated under reduced pressure. The residue was purified by (200-400) silica using the ethyl acetate and cyclohexane (10:90), to give 1-(2,6-difluorophenyl)-5,5,5-trifluoro-pentan-1-one (14.8 g 87.8%) as yellow oil.

¹H NMR (400 MHz, CHLOROFORM-d) δ ppm 7.40 (tt, 1H) 6.91-7.00 (m, 2H) 2.98 (t, 2H) 2.13-2.26 (m, 2H) 1.96-2.05 (m, 2H)

### Step 3: Synthesis of 1-(2-amino-6-fluoro-phenyl)-5,5,5-trifluoro-pentan-1-one

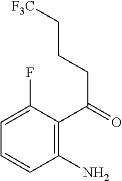 [SMILES: Nc1cccc(F)c1C(=O)CCCC(F)(F)F]

To a solution of 1-(2,6-difluorophenyl)-5,5,5-trifluoro-pentan-1-one (8 g, 31.72 mmol) in acetonitrile (26 mL) added 30% aqueous ammonia solution (210 mL, 1546.5 mmol). This was heated at 120° C. for 10 h in miniclave. The reaction mass was cooled to room temperature, extracted in ethyl acetate (3×500 mL), dried over anhydrous sodium sulfate, filtered and concentrated under reduced pressure. The residue was purified by (200-400) silica using the ethyl acetate and cyclohexane (05:95), obtained 1-(2-amino-6-fluoro-phenyl)-5,5,5-trifluoro-pentan-1-one (4.5 g, 33%) as yellow oil.

¹H NMR (400 MHz, CHLOROFORM-d) δ ppm 7.40 (tt, 1H), 6.91-7.00 (m, 2H), 2.98 (t, 2H), 2.13-2.26 (m, 2H), 1.96-2.05 (m, 2H)

### Step 4: Synthesis of 5-fluoro-2-methyl-4-(4,4,4-trifluorobutyl)quinazoline

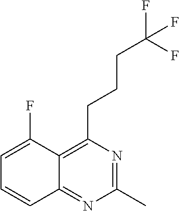 [SMILES: Cc1nc(CCCC(F)(F)F)c2c(F)cccc2n1]

To a solution of 1-(2-amino-6-fluoro-phenyl)-5,5,5-trifluoro-pentan-1-one (2.3 g, 7.4 mmol) in 2,4-pentanedione (0.91 mL, 8.9 mmol) added ammonium acetate (1.7 g, 22 mmol) and then heated to 120° C. for 18 h. The reaction mixture cooled to room temperature, extracted in ethyl acetate (3×200 mL), washed with brine solution (100 mL), dried over anhydrous sodium sulfate, filtered and concentrated under reduced pressure. The residue was purified by (200-400) silica using the ethyl acetate and cyclohexane (10:90), obtained 5-fluoro-2-methyl-4-(4,4,4-trifluorobutyl)quinazoline (1 g, 49%) as yellow solid.

¹H NMR (400 MHz, CHLOROFORM-d) δ ppm 7.71-7.79 (m, 2H), 7.17-7.25 (m, 1H), 3.39 (td, 2H), 2.83 (s, 3H) 2.19-2.36 (m, 2H) 2.07-2.19 (m, 2H).

### Step 5: Synthesis of 5-methoxy-2-methyl-4-(4,4,4-trifluorobutyl)quinazoline

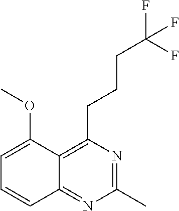 [SMILES: COc1cccc2nc(C)nc(CCCC(F)(F)F)c12]

To a solution of 5-fluoro-2-methyl-4-(4,4,4-trifluorobutyl)quinazoline (1 g, 3.67 mmol) in Methanol (11.02 mL) and N,N-Dimethylformamide (11.02 mL) added 25% solution of sodium methoxide (1.26 mL, 5.51 mmol). The reaction mass was heated to 55° C. for 8 h. The reaction mixture cooled to room temperature, quenched with ice cold water and, extracted in ethyl acetate (3×100 mL), washed with brine solution (100 mL), dried over anhydrous sodium sulfate, filtered and concentrated under reduced pressure. The residue was purified by (200-400) silica using the ethyl acetate and cyclohexane (10:90), obtained 5-methoxy-2-methyl-4-(4,4,4-trifluorobutyl)quinazoline (0.7 g, 67.02%) as light yellow solid.

¹H NMR (400 MHz, CHLOROFORM-d) δ ppm 7.74 (t, 1H), 7.53 (dd, 1H), 6.91 (d, 1H), 4.02 (s, 3H) 3.44-3.51 (m, 2H) 2.82 (s, 3H), 2.22-2.31 (m, 2H), 2.04-2.12 (m, 2H).

### Step 5: Synthesis of 2-methyl-4-(4,4,4-trifluorobutyl)quinazolin-5-ol

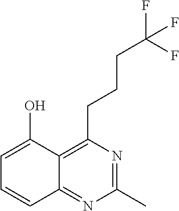 [SMILES: Cc1nc(CCCC(F)(F)F)c2c(O)cccc2n1]

To 5-methoxy-2-methyl-4-(4,4,4-trifluorobutyl)quinazoline (0.1 g, 0.35 mmol), pyridin-1-ium hydrochloride (1.046 g, 8.794 mmol) was added. This was heated at 180° C. for 90 min. The reaction mixture was cooled to room temperature, quenched with ice cold water and extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (25:75), obtained 2-methyl-4-(4,4,4-trifluorobutyl)quinazolin-5-ol (0.03 g, 30.3%) as white solid.

¹H NMR (400 MHz, METHANOL-d4) δ ppm 7.70 (t, 1H), 7.35 (dd, 1H), 6.96 (dd, 1H), 3.49-3.57 (m, 2H), 2.73 (s, 3H), 2.24-2.40 (m, 2H), 2.00-2.11 (m, 2H).

### Step 6: Synthesis of 5-(5-chloropyrimidin-2-yl)oxy-2-methyl-4-(4,4,4-trifluorobutyl)quinazoline (1.013)

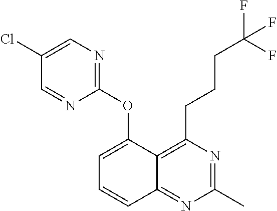 [SMILES: Cc1nc(CCCC(F)(F)F)c2c(Oc3ncc(Cl)cn3)cccc2n1]

To a solution of 2-methyl-4-(4,4,4-trifluorobutyl)quinazolin-5-ol (25 mg, 0.092 mmol) in DMF (0.5 mL) added Potassium carbonate (0.038 g, 0.2776 mmol) and 5-chloro-2-methylsulfonyl-pyrimidine (0.022 g, 0.111 mmol). This was heated to 50° C. for 2 h. The reaction mixture cooled to room temperature, quenched with ice cold water and, extracted in ethyl acetate (3×20 mL), washed with brine solution (20 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-2-methyl-4-(4,4,4-trifluorobutyl)quinazoline (24.5 mg, 69.2%) as white solid.

¹H NMR (400 MHz, CHLOROFORM-d) δ ppm 8.53 (s, 2H), 7.91 (dd, 1H), 7.85 (t, 1H), 7.29 (s, 1H) 3.32 (t, 2H), 2.85 (s, 3H), 2.14-2.26 (m, 2H), 1.97-2.07 (m, 2H).

### EXAMPLE 7: SYNTHESIS OF 8-CHLORO-5-(5-CHLOROPYRIMIDIN-2-YL)OXY-2-METHYL-4-(4,4,4-TRIFLUOROBUTYL)QUINAZOLINE (1.019)

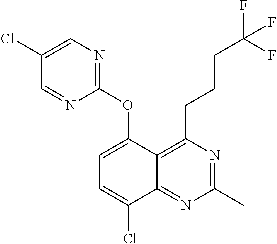 [SMILES: Cc1nc(CCCC(F)(F)F)c2c(Oc3ncc(Cl)cn3)ccc(Cl)c2n1]

### Step 1: Synthesis of 8-bromo-5-methoxy-2-methyl-4-(4,4,4-trifluorobutyl) quinazoline

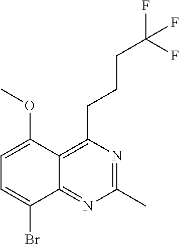 [SMILES: COc1ccc(Br)c2nc(C)nc(CCCC(F)(F)F)c12]

To a solution of 5-methoxy-2-methyl-4-(4,4,4-trifluorobutyl)quinazoline (0.5 g, 1.759 mmol) in acetonitrile (5.3 mL)) added N-bromosuccinimide (0.313 g, 1.76 mmol) at room temperature. This was stirred at room temperature for 1 h. The reaction mixture quenched with ice cold water and extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (10:90), obtained 8-bromo-5-methoxy-2-methyl-4-(4,4,4-trifluorobutyl)quinazoline (0.56 g, 87.67%) as yellow solid.

¹H NMR (400 MHz, CHLOROFORM-d) δ ppm 8.04 (d, 1H), 6.81 (d, 1H), 4.01 (s, 3H), 3.45-3.52 (m, 2H), 2.88 (s, 3H), 2.20-2.33 (m, 2H), 2.02-2.11 (m, 2H)

### Step 2: Synthesis of 8-chloro-2-methyl-4-(4,4,4-trifluorobutyl)quinazolin-5-ol

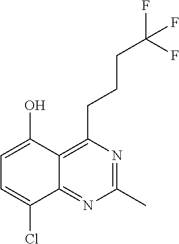 [SMILES: Cc1nc(CCCC(F)(F)F)c2c(O)ccc(Cl)c2n1]

To 8-bromo-5-methoxy-2-methyl-4-(4,4,4-trifluorobutyl)quinazoline (0.2 g, 0.55 mmol) pyridin-1-ium hydrochloride (1.64 g, 13.77 mmol) was added. This was heated at 180° C. for 90 min. The reaction mixture cooled to room temperature, quenched with ice cold water and, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 8-chloro-2-methyl-4-(4,4,4-trifluorobutyl)quinazolin-5-ol (0.04 g, 23.84%) as off white solid.

¹H NMR (400 MHz, METHANOL-d4) δ ppm 7.80 (d, 1H), 6.92 (d, Hz, 1H), 3.50-3.58 (m, 2H), 2.79 (s, 3H), 2.24-2.38 (m, 2H), 2.01-2.11 (m, 2H).

### Step 3: Synthesis of 8-chloro-2-methyl-4-(4,4,4-trifluorobutyl)quinazolin-5-ol (1.019)

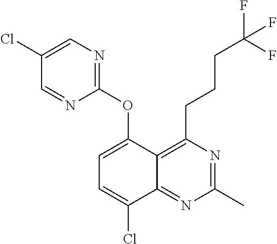 [SMILES: Cc1nc(CCCC(F)(F)F)c2c(Oc3ncc(Cl)cn3)ccc(Cl)c2n1]

To a solution of 8-chloro-2-methyl-4-(4,4,4-trifluorobutyl)quinazolin-5-ol (23 mg, 0.074 mmol) in DMF (0.5 mL) added Potassium carbonate (0.031 g, 0.22 mmol) and 5-chloro-2-methylsulfonyl-pyrimidine (0.013 g, 0.067 mmol). This was heated to 40° C. for 3 h. The reaction mixture cooled to room temperature, quenched with ice cold water and, extracted in ethyl acetate (3×20 mL), washed with brine solution (20 mL), dried over anhydrous sodium sulfate, filtered and concentrated under reduced pressure. The residue was purified by (200-400) silica using the ethyl acetate and cyclohexane (10:90), obtained 8-chloro-5-(5-chloropyrimidin-2-yl)oxy-2-methyl-4-(4,4,4-trifluorobutyl)quinazoline (15 mg, 48.6%) as yellow solid.

¹H NMR (400 MHz, CHLOROFORM-d) δ ppm 8.53 (s, 2H), 7.96 (d, 1H), 7.22-7.25 (d, 1H), 3.33 (t, 2H), 2.92 (s, 3H), 2.14-2.26 (m, 2H), 1.97-2.08 (m, 2H)

### EXAMPLE 8: SYNTHESIS OF 5-(5-CHLOROPYRIMIDIN-2-YL)OXY-2,8-DIMETHYL-4-(4,4,4-TRIFLUOROBUTYL)QUINAZOLINE (1.017)

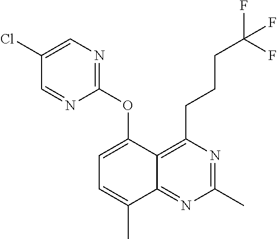 [SMILES: Cc1nc(CCCC(F)(F)F)c2c(Oc3ncc(Cl)cn3)ccc(C)c2n1]

### Step 1: Synthesis of 5-methoxy-2,8-dimethyl-4-(4,4,4-trifluorobutyl)quinazoline

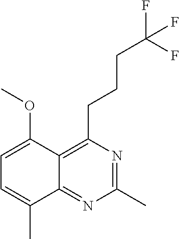 [SMILES: COc1ccc(C)c2nc(C)nc(CCCC(F)(F)F)c12]

To 8-bromo-5-methoxy-2-methyl-4-(4,4,4-trifluorobutyl)quinazoline (0.21 g, 0.58 mmol) (0.2 g, 0.55 mmol) in 1,4-dioxane (12.6 mL) added methylboronic acid (0.111 g, 1.85 mmol) and cesium carbonate (0.61 g, 1.85 mmol). After degassing for 15 minutes added [1,1′-Bis(Diphenylphosphino)Ferrocene]DichloroPalladium (II) (0.051 g, 0.069 mmol). This was heated at 100° C. for 60 min in microwave. The reaction mixture cooled to room temperature, quenched with water and extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 5-methoxy-2,8-dimethyl-4-(4,4,4-trifluorobutyl)quinazoline (0.14 g, 81.17%) as off white solid.

¹H NMR (400 MHz, CHLOROFORM-d) δ ppm 7.57 (dd, 1H), 6.80 (d, 1H), 3.98 (s, 3H), 3.42-3.49 (m, 2H), 2.83 (s, 3H), 2.63 (s, 3H), 2.20-2.32 (m, 2H), 2.00-2.11 (m, 2H)

### Step 2: Synthesis of 2,8-dimethyl-4-(4,4,4-trifluorobutyl)quinazolin-5-ol

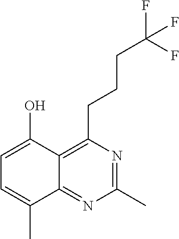 [SMILES: Cc1nc(CCCC(F)(F)F)c2c(O)ccc(C)c2n1]

To 5-methoxy-2,8-dimethyl-4-(4,4,4-trifluorobutyl)quinazoline (0.14 g, 0.47 mmol) pyridin-1-ium;hydrochloride (1.36 g, 11.73 mmol) was added. This was heated at 180° C. for 90 min. The reaction mixture cooled to room temperature, quenched with ice cold water and, extracted in ethyl acetate (3×50 mL), washed with brine solution (50 mL), dried over anhydrous sodium sulfate, filtered and concentrated under reduced pressure. The residue was purified by reverse phase column chromatography (C₁₈ column) using the acetonitrile and water (10:90), obtained 2,8-dimethyl-4-(4,4,4-trifluorobutyl)quinazolin-5-ol (0.06 g, 45%) as off white solid.

¹H NMR (400 MHz, METHANOL-d4) δ ppm 7.45 (dd, 1H), 6.78 (d, 1H), 3.41-3.46 (m, 2H), 2.72 (s, 3H), 2.50 (s, 3H), 2.19-2.36 (m, 2H) 1.92-2.06 (m, 2H)

### Step-3: Synthesis of 5-(5-chloropyrimidin-2-yl)oxy-2,8-dimethyl-4-(4,4,4-trifluorobutyl)quinazoline (1.017)

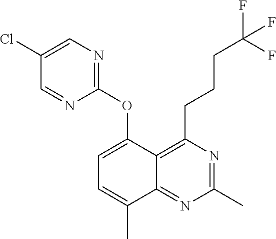 [SMILES: Cc1nc(CCCC(F)(F)F)c2c(Oc3ncc(Cl)cn3)ccc(C)c2n1]

To a solution of 2,8-dimethyl-4-(4,4,4-trifluorobutyl)quinazolin-5-ol (42 mg, 0.148 mmol) in dimethyl formamide (0.8 mL) added Potassium carbonate (0.061 g, 0.44 mmol) and 5-chloro-2-methylsulfonyl-pyrimidine (0.026 g, 0.148 mmol). This was heated to 40° C. for 1 h. The reaction mixture cooled to room temperature, quenched with ice cold water and, extracted in ethyl acetate (3×30 mL), washed with brine solution (20 mL), dried over anhydrous sodium sulfate filtered and concentrated under reduced pressure. The residue was purified by (200-400) silica using the ethyl acetate and cyclohexane (10:90), obtained 5-(5-chloropyrimidin-2-yl)oxy-2,8-dimethyl-4-(4,4,4-trifluorobutyl)quinazoline (0.041 g, 69.94%) as off white solid.

¹H NMR (400 MHz, CHLOROFORM-d) δ ppm 8.51 (s, 2H), 7.68 (d, 1H), 7.16 (d, 1H), 3.29 (t, 2H), 2.85 (s, 3H), 2.75 (s, 3H), 2.12-2.25 (m, 2H), 1.95-2.04 (m, 2H).

### EXAMPLE 9 SYNTHESIS OF 8-BROMO-5-(5-CHLOROPYRIMIDIN-2-YL)OXY-4-(4,4,4-TRIFLUOROBUTYL)-2-(TRIFLUOROMETHYL)QUINAZOLINE (1.048)

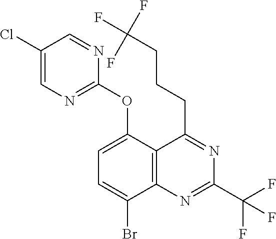 [SMILES: FC(F)(F)CCCc1nc(C(F)(F)F)nc2c(Br)ccc(Oc3ncc(Cl)cn3)c12]

### Step-1: Synthesis of 5-methoxy-2-(trifluoromethyl)-3H-quinazolin-4-one

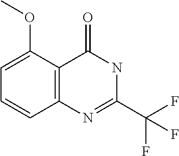

To a stirred mixture of 2-amino-6-methoxy-benzoic acid (25.00 g, 149.55 mmol) and 4-dimethylaminopyridine (1.85 g, 14.95 mmol) in toluene (450 mL) at 0° C. was added trifluoroacetic acid anhydride (66.82 mL, 463.62 mmol) followed by dropwise addition of triethylamine (92.4 mL, 658.04 mmol). The reaction mixture was allowed to warm to room temperature and stirred overnight. Then ammonium acetate (20.92 g, 263.22 mmol) was added and reaction was heated at 110° C. for 18 h. The reaction was allowed to cool to room temperature, diluted with ice cold water (1000 mL) and stirred vigorously. The resultant solid was filtered, washed with water and methy tertiary butyl ether and vacuum dried to afford 5-methoxy-2-(trifluoromethyl)-3H-quinazolin-4-one (30.4 g, 82%) as a cream solid.

¹H NMR (400 MHz, DMSO-d6) δ ppm 7.80 (t, 1H), 7.32 (d, 1H), 7.20 (d, 1H), 3.90 (s, 3H).

### Step-2: Synthesis of 4-bromo-5-methoxy-2-(trifluoromethyl)quinazoline

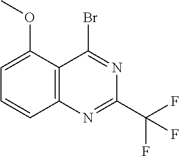 [SMILES: COc1cccc2nc(C(F)(F)F)nc(Br)c12]

To a stirred mixture of 5-methoxy-2-(trifluoromethyl)quinazolin-4-ol (8.0 g, 33 mmol) in toluene (30 mL) added phosphorus oxybromide (14 g, 49 mmol). This was heated to 140° C. for 3 h. The reaction mixture was allowed to cool to room temperature and poured into saturated sodium bicarbonate solution, extracted in ethyl acetate (3×500 mL), washed with brine solution (200 mL), dried over anhydrous sodium sulfate, filtered and concentrated under reduced pressure. The residue was purified by (200-400) silica using the ethyl acetate and cyclohexane (5:95), obtained 4-bromo-5-methoxy-2-(trifluoromethyl)quinazoline (5.5 g, 55%) as light yellow solid.

¹H NMR (400 MHz, CHLOROFORM-d) δ ppm 7.95 (t, 1H), 7.76 (d, 1H), 7.16 (d, 1H), 4.06 (s, 3H).

### Step-3: Synthesis of 5-methoxy-4-(4,4,4-trifluorobutyl)-2-(trifluoromethyl) quinazoline

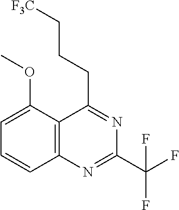 [SMILES: COc1cccc2nc(C(F)(F)F)nc(CCCC(F)(F)F)c12]

To stirred magnesium turnings (1.018 g, 41.88 mmol) in tetrahydrofuran (25 mL) was added a crystal of iodine. The reaction was stirred for 10 mins and heated to reflux. Then a small amount of 1-bromo-4,4,4-trifluorobutane in tetrahydrofuran was added. The heating was continued until the solution went from yellow to grey in colour. The remaining 1-bromo-4,4,4-trifluorobutane (4.0 g, 20.94 mmol) solution in tetrahydrofuran (25 mL) was added dropwise under heating condition and then the reaction heated at 75° C. for 1 h, then allowed to cool to room temperature. The above solution of bromo(4,4,4-trifluorobutyl)magnesium in tetrahydrofuran was added dropwise to ice cooled solution of 4-bromo-5-methoxy-2-(trifluoromethyl)quinazoline (2.0 g, 6.51 mmol) and Copper(I) iodide (0.027 g, 0.105 mmol) in tetrahydrofuran (16 mL) at 0° C. and the reaction was stirred at room temperature for 16 h. The reaction was quenched with saturated aqueous ammonium chloride and extracted with ethyl acetate (3×500 mL), the combined organics were washed with brine (200 mL), dried over sodium sulfate, filtered and evaporated to dryness under reduced pressure to give 5-methoxy-4-(4,4,4-trifluorobutyl)-2-(trifluoromethyl)quinazoline (2.1 g, 95%).

¹H NMR (400 MHz, CHLOROFORM-d) δ ppm 7.88 (t, 1H), 7.74 (dd, 1H), 7.09 (d, 1H), 4.06 (s, 3H), 3.59 (t, 2H), 2.23-2.36 (m, 2H), 2.07-2.21 (m, 2H).

### Step-4: Synthesis of 8-bromo-5-methoxy-4-(4,4,4-trifluorobutyl)-2-(trifluoromethyl) quinazoline

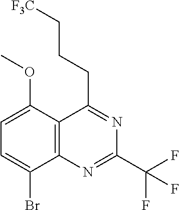 [SMILES: COc1ccc(Br)c2nc(C(F)(F)F)nc(CCCC(F)(F)F)c12]

To a solution of 5-methoxy-4-(4,4,4-trifluorobutyl)-2-(trifluoromethyl)quinazoline (1.75 g, 5.17 mmol) in acetonitrile (15.5 mL)) added N-bromosuccinimide (0.921 g, 5.17 mmol) at room temperature. This was stirred at room temperature for 1 h. The reaction mixture was quenched with ice cold water and extracted in ethyl acetate (3×200 mL), washed with brine solution (100 mL), dried over anhydrous sodium sulfate, filtered and concentrated under reduced pressure. The residue was purified by (200-400) silica using the ethyl acetate and cyclohexane (10:90), obtained 8-bromo-5-methoxy-4-(4,4,4-trifluorobutyl)-2-(trifluoromethyl)quinazoline (1.51 g, 53.3%) as yellow solid.

¹H NMR (400 MHz, CHLOROFORM-d) δ ppm 8.15 (d, 1H), 6.99 (d, 1H), 4.07 (s, 3H), 3.57-3.64 (m, 2H), 2.31 (dt, 2H), 2.11-2.18 (m, 2H)

### Step 5: Synthesis of 8-bromo-4-(4,4,4-trifluorobutyl)-2-(trifluoromethyl) quinazolin-5-ol

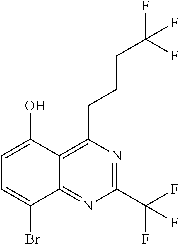 [SMILES: Oc1ccc(Br)c2nc(C(F)(F)F)nc(CCCC(F)(F)F)c12]

To a stirred solution of 1-Dodecanethiol (2.877 mmol) in N,N-Dimethylformamide (3 mL) was added Lithium tert-butoxide (0.235 g, 2.877 mmol). After stirring for 5 minutes, added a solution of 8-bromo-5-methoxy-4-(4,4,4-trifluorobutyl)-2-(trifluoromethyl)quinazoline (600 mg, 1.439 mmol) in N,N-Dimethylformamide (1 mL). The reaction was heated at 100° C. for 4 hours. The reaction mixture was cooled to room temperature, quenched with ice cold water, acidified with 2 N hydrochloric acid solution and extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (20:80), obtained 8-bromo-4-(4,4,4-trifluorobutyl)-2-(trifluoromethyl)quinazolin-5-ol (0.30 g, 51.74%) as brown solid.

¹H NMR (400 MHz, METHANOL-d4) δ ppm 8.11 (d, 1H), 7.06 (d, 1H), 3.68 (t, 2H), 2.28-2.41 (m, 2H), 2.08-2.17 (m, 2H).

### Step-6: Synthesis of 8-bromo-5-(5-chloropyrimidin-2-yl)oxy-4-(4,4,4-trifluorobutyl)-2-(trifluoromethyl)quinazoline (1.048)

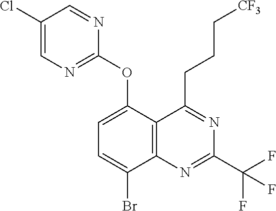 [SMILES: FC(F)(F)CCCc1nc(C(F)(F)F)nc2c(Br)ccc(Oc3ncc(Cl)cn3)c12]

To a solution of 8-bromo-4-(4,4,4-trifluorobutyl)-2-(trifluoromethyl)quinazolin-5-ol (0.30 g, 0.744 mmol) in isopropyl alcohol (3 mL) added Potassium carbonate (0.309 g, 2.23 mmol) and 5-chloro-2-methylsulfonyl-pyrimidine (0.302 g, 1.49 mmol). This was heated to 55° C. for 18 h. The reaction mixture cooled to room temperature, quenched with ice cold water and, extracted in ethyl acetate (3×50 mL), washed with brine solution (20 mL), dried over anhydrous sodium sulfate, filtered and concentrated under reduced pressure. The residue was purified by (200-400) silica using the ethyl acetate and cyclohexane (10:90), obtained 8-bromo-5-(5-chloropyrimidin-2-yl)oxy-4-(4,4,4-trifluorobutyl)-2-(trifluoromethyl)quinazoline (0.178 g, 46.39%) as off white solid.

¹H NMR (400 MHz, CHLOROFORM-d) δ ppm 8.54 (s, 2H), 8.32 (m, 1H), 7.41 (m, 1H), 3.48 (t, 2H), 2.20-2.32 (m, 2H), 2.07-2.20 (m, 2H)

### EXAMPLE 10: SYNTHESIS OF 8-BROMO-5-(5-CHLOROPYRIMIDIN-2-YL)OXY-4-(4,4,4-TRIFLUOROBUTYL)-2-(TRIFLUOROMETHYL)QUINAZOLINE (1.047)

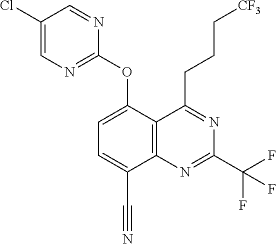 [SMILES: N#Cc1ccc(Oc2ncc(Cl)cn2)c2c(CCCC(F)(F)F)nc(C(F)(F)F)nc12]

### Step-1: Synthesis of 8-bromo-5-(5-chloropyrimidin-2-yl)oxy-4-(4,4,4-trifluorobutyl)-2-(trifluoromethyl)quinazoline (1.047)

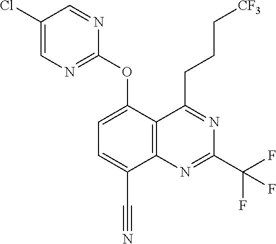 [SMILES: N#Cc1ccc(Oc2ncc(Cl)cn2)c2c(CCCC(F)(F)F)nc(C(F)(F)F)nc12]

To a solution of 8-bromo-5-(5-chloropyrimidin-2-yl)oxy-4-(4,4,4-trifluorobutyl)-2-(trifluoromethyl)quinazoline (1.048; 0.09 g, 0.175 mmol) in N, N-dimethylformamide (1.75 mL) was added 4,5-bis(diphenylphosphino)-9,9-dimethylxanthene (0.028 g, 0.0349 mmol), Tris(dibenzylideneacetone) dipalladium (0) (0.0165 g, 0.0175 mmol) followed by zinc cyanide (0.0307 mg, 0.26 mmol). The reaction mixture was heated at 90° C. for 4 h. Then quenched in water (20 mL), extracted in ethyl acetate (3×50 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-4-(4,4,4-trifluorobutyl)-2-(trifluoromethyl)quinazoline-8-carbonitrile (0.023 g, 28.53%) as off white solid.

¹H NMR (400 MHz, CHLOROFORM-d) δ ppm 8.59 (s, 2H), 8.39 (d, 1H), 7.61 (d, 1H), 3.55 (t, 2H), 2.20-2.32 (m, 2H), 2.08-2.20 (m, 2H)

### EXAMPLE 11: SYNTHESIS OF 5-(5-CHLOROPYRIMIDIN-2-YL)OXY-8-METHYL-4-(4,4,4-TRIFLUOROBUTYL)-2-(TRIFLUOROMETHYL)QUINAZOLINE (1.046)

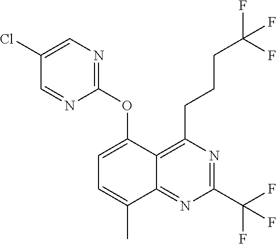 [SMILES: Cc1ccc(Oc2ncc(Cl)cn2)c2c(CCCC(F)(F)F)nc(C(F)(F)F)nc12]

### Step 1: Synthesis of 5-methoxy-8-methyl-4-(4,4,4-trifluorobutyl)-2-(trifluoromethyl)quinazoline

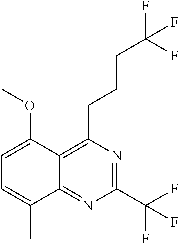 [SMILES: COc1ccc(C)c2nc(C(F)(F)F)nc(CCCC(F)(F)F)c12]

To 8-bromo-5-methoxy-4-(4,4,4-trifluorobutyl)-2-(trifluoromethyl)quinazoline (0.32 g, 0.767 mmol) in 1,4-dioxane (5 mL) added methylboronic acid (0.147 g, 2.45 mmol) and cesium carbonate (0.80 g, 2.45 mmol). After degassing for 15 minutes added [1,1′-Bis(Diphenylphosphino)Ferrocene]DichloroPalladium (II) (0.067 g, 0.092 mmol). This was heated at 100° C. for 90 min. The reaction mixture was cooled to room temperature, quenched with water and extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 5-methoxy-8-methyl-4-(4,4,4-trifluorobutyl)-2-(trifluoromethyl)quinazoline (0.232 g, 85.84%) as off white solid.

¹H NMR (400 MHz, CHLOROFORM-d) δ ppm 7.72 (d, 1H), 7.00 (d, 1H), 4.03 (s, 3H) 3.57-3.63 (m, 2H), 2.69 (s, 3H), 2.30 (m, 2H), 2.09-2.18 (m, 2H)

### Step 2: Synthesis of 8-methyl-4-(4,4,4-trifluorobutyl)-2-(trifluoromethyl) quinazolin-5-ol

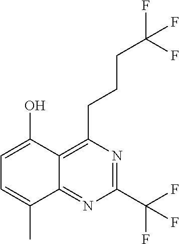 [SMILES: Cc1ccc(O)c2c(CCCC(F)(F)F)nc(C(F)(F)F)nc12]

To 5-methoxy-8-methyl-4-(4,4,4-trifluorobutyl)-2-(trifluoromethyl)quinazoline (0.19 g, 0.54 mmol) pyridin-1-ium;hydrochloride (1.60 g, 13.48 mmol) was added. This was heated at 190° C. for 2 h. The reaction mixture cooled to room temperature, quenched with ice cold water and, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 8-methyl-4-(4,4,4-trifluorobutyl)-2-(trifluoromethyl)quinazolin-5-ol (0.11 g, 57.9%) as off white solid.

¹H NMR (400 MHz, METHANOL-d4) δ ppm 7.52 (d, 1H), 6.92 (d, 1H), 3.55 (t, 2H), 2.52 (s, 3H), 2.22-2.37 (m, 2H), 1.99-2.12 (m, 2H).

### Step-3: Synthesis of 5-(5-chloropyrimidin-2-yl)oxy-8-methyl-4-(4,4,4-trifluorobutyl)-2-(trifluoromethyl)quinazoline (1.046)

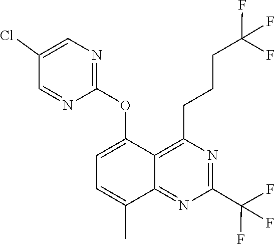 [SMILES: Cc1ccc(Oc2ncc(Cl)cn2)c2c(CCCC(F)(F)F)nc(C(F)(F)F)nc12]

To a solution of 8-methyl-4-(4,4,4-trifluorobutyl)-2-(trifluoromethyl)quinazolin-5-ol (0.07 mg, 0.207 mmol) in N,N-Dimethylformamide (0.8 mL) added Potassium carbonate (0.0858 g, 0.62 mmol) and 5-chloro-2-methylsulfonyl-pyrimidine (0.063 g, 0.31 mmol). This was heated to 50° C. for 18 h. The reaction mixture cooled to room temperature, quenched with ice cold water and, extracted in ethyl acetate (3×30 mL), washed with brine solution (20 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-8-methyl-4-(4,4,4-trifluorobutyl)-2-(trifluoromethyl)quinazoline (66 mg, 70.74%) as off white solid.

¹H NMR (400 MHz, CHLOROFORM-d) δ ppm 8.53 (s, 2H), 7.86 (dd, 1H), 7.40 (d, 1H), 3.44 (t, 2H), 2.83 (s, 3H), 2.17-2.30 (m, 2H), 2.05-2.14 (m, 2H)

### EXAMPLE 12: SYNTHESIS OF 5-(5-CHLOROPYRIMIDIN-2-YL)OXY-2,7-DIMETHYL-4-(4,4,4-TRIFLUOROBUTYL)QUINAZOLINE (1.016)

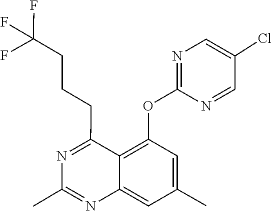 [SMILES: Cc1cc(Oc2ncc(Cl)cn2)c2c(CCCC(F)(F)F)nc(C)nc2c1]

### Step 1: Synthesis of 5-methoxy-2,7-dimethyl-4-(4,4,4-trifluorobutyl)quinazoline

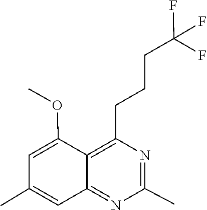 [SMILES: COc1cc(C)cc2nc(C)nc(CCCC(F)(F)F)c12]

To 7-bromo-5-methoxy-2-methyl-4-(4,4,4-trifluorobutyl)quinazoline (0.45 g, 1.24 mmol) in 1,4-dioxane (27 mL) added methylboronic acid (0.24 g, 3.96 mmol) and cesium carbonate (1.29 g, 3.96 mmol). After degassing for 15 minutes added [1,1′-Bis(Diphenylphosphino)Ferrocene]DichloroPalladium (II) 0.109 g, 0.149 mmol). This was heated at 100° C. for 4 h. The reaction mixture cooled to room temperature, quenched with water and extracted in ethyl acetate (3×100 mL), washed with brine solution (50 mL), dried over anhydrous sodium sulfate, filtered and concentrated under reduced pressure. The residue was purified by (200-400) silica using the ethyl acetate and cyclohexane (15:85), obtained 5-methoxy-2,7-dimethyl-4-(4,4,4-trifluorobutyl)quinazoline (0.28 g, 75.76%) as off white solid.

¹H NMR (400 MHz, CHLOROFORM-d) δ ppm 7.34 (s, 1H), 6.73 (s, 1H) 4.01 (s, 3H) 3.41-3.48 (m, 2H), 2.80 (s, 3H), 2.54 (s, 3H), 2.27 (br s, 2H), 2.07 (br d, 2H)

### Step 2: Synthesis of 2,7-dimethyl-4-(4,4,4-trifluorobutyl)quinazolin-5-ol

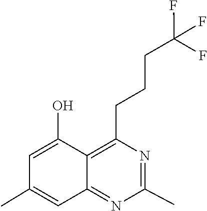 [SMILES: Cc1cc(O)c2c(CCCC(F)(F)F)nc(C)nc2c1]

To 5-methoxy-2,8-dimethyl-4-(4,4,4-trifluorobutyl)quinazoline (0.24 g, 0.805 mmol) pyridin-1-ium;hydrochloride (2.39 g, 20.11 mmol) was added. This was heated at 190° C. for 1 h. The reaction mixture cooled to room temperature, quenched with ice cold water and, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (30:70), obtained 2,7-dimethyl-4-(4,4,4-trifluorobutyl)quinazolin-5-ol (0.14 g, 58.74%) as light brown solid.

¹H NMR (400 MHz, METHANOL-d4) δ ppm 7.15 (s, 1H), 6.81 (d, 1H), 3.45-3.53 (m, 2H), 2.70 (s, 3H), 2.45 (s, 3H), 2.23-2.36 (m, 2H), 1.98-2.11 (m, 2H)

### Step-3: Synthesis of 5-(5-chloropyrimidin-2-yl)oxy-2,7-dimethyl-4-(4,4,4-trifluorobutyl)quinazoline (1.016)

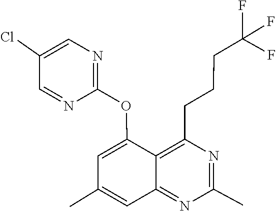 [SMILES: Cc1cc(Oc2ncc(Cl)cn2)c2c(CCCC(F)(F)F)nc(C)nc2c1]

To a solution of 2,8-dimethyl-4-(4,4,4-trifluorobutyl)quinazolin-5-ol (60 mg, 0.211 mmol) in N,N-Dimethylformamide (1.2 mL) added Potassium carbonate (0.088 g, 0.63 mmol) and 5-chloro-2-methylsulfonyl-pyrimidine (0.048 g, 0.253 mmol). This was heated to 50° C. for 2 h. The reaction mixture cooled to room temperature, quenched with ice cold water and extracted in ethyl acetate (3×30 mL), washed with brine solution (20 mL), dried over anhydrous sodium sulfate filtered and concentrated under reduced pressure. The residue was purified by (200-400) silica using the ethyl acetate and cyclohexane (10:90), obtained 5-(5-chloropyrimidin-2-yl)oxy-2,7-dimethyl-4-(4,4,4-trifluorobutyl)quinazoline (0.040 g, 48.6%) as off white solid.

¹H NMR (400 MHz, CHLOROFORM-d) δ ppm 8.55 (s, 2H), 7.70 (s, 1H), 7.12 (s, 1H), 3.28 (t, 2H), 2.84 (s, 3H), 2.57 (s, 3H), 2.12-2.26 (m, 2H), 1.97-2.06 (m, 2H)

### EXAMPLE 13: SYNTHESIS OF 5-(5-CHLOROPYRIMIDIN-2-YL)OXY-2-METHYL-4-(4,4,4-TRIFLUOROBUTYL)QUINOLINE (1.035)

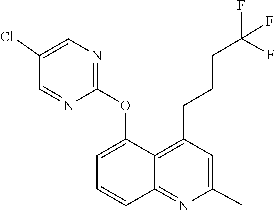 [SMILES: Cc1cc(CCCC(F)(F)F)c2c(Oc3ncc(Cl)cn3)cccc2n1]

### Step 1: Synthesis of 5-fluoro-2-methyl-4-(4,4,4-trifluorobutyl)quinoline

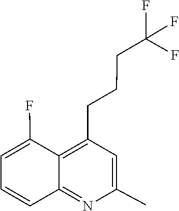 [SMILES: Cc1cc(CCCC(F)(F)F)c2c(F)cccc2n1]

To 1-(2-amino-6-fluoro-phenyl)-5,5,5-trifluoro-pentan-1-one (0.45 g, 1.806 mmol) in ethanol (9.0 mL) added acetone (0.66 mL, 9.03 mmol) and potassium hydroxide (0.21 g, 3.61 mmol). This was heated to 85° C. for 5 h. The reaction mixture cooled to room temperature, quenched with ice cold water and, extracted in ethyl acetate (3×100 mL), washed with brine solution (50 mL), dried over anhydrous sodium sulfate, filtered and concentrated under reduced pressure. The residue was purified by (200-400) silica using the ethyl acetate and cyclohexane (5:95), obtained 5-fluoro-2-methyl-4-(4,4,4-trifluorobutyl)quinoline (0.31 g, 63.3%) as yellow oil.

¹H NMR (400 MHz, CHLOROFORM-d) δ ppm 7.84 (d, 1H), 7.58 (td, 1H), 7.16 (ddd, 1H), 7.10 (s, 1H), 3.19 (td, 2H), 2.70 (s, 3H), 2.15-2.27 (m, 2H), 1.94-2.05 (m, 2H)

### Step 2: Synthesis of 5-methoxy-2-methyl-4-(4,4,4-trifluorobutyl)quinoline

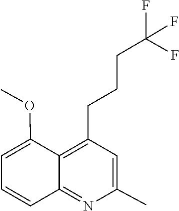 [SMILES: COc1cccc2nc(C)cc(CCCC(F)(F)F)c12]

To a solution of 5-fluoro-2-methyl-4-(4,4,4-trifluorobutyl)quinoline (0.29 g, 1.069 mmol) in Methanol (2.6 mL) and N,N-Dimethylformamide (2 mL) added 25% solution of sodium methoxide (0.37 mL, 1.60 mmol). The reaction mass was heated to 90° C. for 18 h. The reaction mixture cooled to room temperature, quenched with ice cold water and, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (10:90), obtained 5-methoxy-2-methyl-4-(4,4,4-trifluorobutyl)quinoline (0.18 g, 59.42%) as light yellow oil.

¹H NMR (400 MHz, CHLOROFORM-d) δ ppm 7.64 (dd, 1H), 7.55 (t, 1H), 7.00 (s, 1H), 6.84 (d, 1H), 3.96 (s, 3H), 3.23-3.29 (m, 2H), 2.66 (s, 3H), 2.13-2.24 (m, 2H), 1.91-1.98 (m, 2H).

### Step 3: Synthesis of 2-methyl-4-(4,4,4-trifluorobutyl)quinolin-5-ol

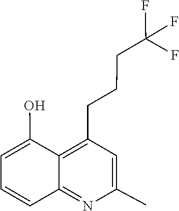 [SMILES: Cc1cc(CCCC(F)(F)F)c2c(O)cccc2n1]

To 5-methoxy-2-methyl-4-(4,4,4-trifluorobutyl)quinoline (0.16 g, 0.565 mmol) pyridin-1-ium;hydrochloride (1.68 g, 14.12 mmol) was added. This was heated at 180° C. for 2 h. The reaction mixture cooled to room temperature, quenched with ice cold water and then with 2N hydrochloric acid, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 2-methyl-4-(4,4,4-trifluorobutyl)quinolin-5-ol (0.075 g, 47.34%) as gummy mass.

¹H NMR (400 MHz, METHANOL-d4) δ ppm 7.41-7.49 (m, 2H), 7.12 (s, 1H), 6.86 (dd, 1H), 3.33-3.39 (m, 2H), 2.62 (s, 3H), 2.15-2.37 (m, 2H), 1.90-2.07 (m, 2H).

### Step 4: Synthesis of 5-(5-chloropyrimidin-2-yl)oxy-2-methyl-4-(4,4,4-trifluorobutyl)quinoline (1.035)

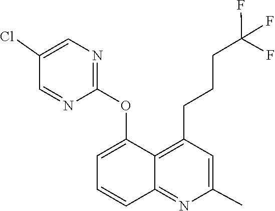 [SMILES: Cc1cc(CCCC(F)(F)F)c2c(Oc3ncc(Cl)cn3)cccc2n1]

To a solution of 2-methyl-4-(4,4,4-trifluorobutyl)quinolin-5-ol (25 mg, 0.093 mmol) in N,N-Dimethylformamide (0.5 mL) added Potassium carbonate (0.019 g, 0.14 mmol) and 5-chloro-2-methylsulfonyl-pyrimidine (0.018 g, 0.093 mmol). This was heated to 50° C. for 1 h. The reaction mixture cooled to room temperature, quenched with ice cold water and, extracted in ethyl acetate (3×30 mL), washed with brine solution (20 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-2-methyl-4-(4,4,4-trifluorobutyl)quinoline (24 mg, 48.6%) as off white solid.

¹H NMR: (400 MHz, CHLOROFORM-d) δ ppm 8.50 (s, 2H), 7.99 (d, 1H), 7.67 (t, 1H), 7.21 (d, 1H), 7.08 (s, 1H), 3.02-3.11 (m, 2H), 2.70 (s, 3H), 2.08-2.22 (m, 2H), 1.81-1.96 (m, 2H).

### EXAMPLE 14: SYNTHESIS OF 5-(5-CHLOROPYRIMIDIN-2-YL)OXY-4-(4,4,4-TRIFLUOROBUTYL)-2-(TRIFLUOROMETHYL)QUINOLINE (1.015)

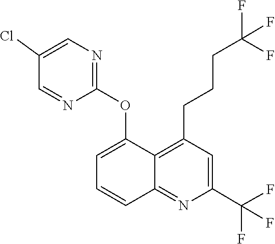 [SMILES: FC(F)(F)CCCc1cc(C(F)(F)F)nc2cccc(Oc3ncc(Cl)cn3)c12]

### Step 1: Synthesis of 8-bromo-5-methoxy-2-(trifluoromethyl)-1H-quinolin-4-one

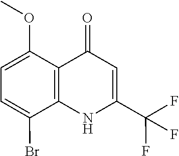 [SMILES: COc1ccc(Br)c2[nH]c(C(F)(F)F)cc(=O)c12]

A mixture of ethyl-4,4,4-trifluoroacetoacetate (4.3283 g, 23.51 mmol) and polyphosphoric acid (9.76 mL) was heated to 100° C. for 15 mins. To this reaction mass, 2-bromo-5-methoxyaniline (5 g, 23.51 mmol) was added drop wise. The reaction mass was refluxed at 150° C. for 3 h. The reaction mixture cooled to room temperature and slowly poured into a solution of sodium hydroxide (20 g in 100 mL water). The solid formed was filtered off. The filtrate was acidified using concentrated hydrochloric acid. The solid formed was filtered, washed with water, and dried under vacuum to give 8-bromo-5-methoxy-2-(trifluoromethyl)-1H-quinolin-4-one (3.48 g, 46.0%) as off white solid.

¹H NMR (400 MHz, DMSO-d6) δ ppm 8.09 (d, 1H), 7.28 (s, 1H), 7.03 (d, 1H), 3.99 (s, 3H)

### Step 2: Synthesis of 5-methoxy-2-(trifluoromethyl)quinolin-4-ol

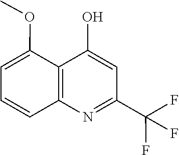 [SMILES: COc1cccc2nc(C(F)(F)F)cc(O)c12]

To a solution of 8-bromo-5-methoxy-2-(trifluoromethyl)quinolin-4-ol (1.9 g, 5.9 mmol) in tetrahydrofuran (19 mL), added n-butyl lithium (2.0 mol/L) in hexane (8.8 mL, 18 mmol) dropwise at −78° C. After stirring at −70° C. for 1 h, added methanol (5 mL/g, 9.5 mL). The reaction mass was stirred at −70° C. for 1 h and slowly increased t to room temperature, stirred for 3 h and then cooled to 5° C., quenched with saturated solution of ammonium chloride, extracted in ethyl acetate (3×200 mL), washed with brine solution (50 mL), dried over anhydrous sodium sulfate, filtered and concentrated under reduced pressure to afford 5-methoxy-2-(trifluoromethyl)quinolin-4-ol (1.5 g, 100%) as off white solid.

¹H NMR (400 MHz, CHLOROFORM-d) δ ppm 9.89-10.12 (m, 1H), 7.79 (dd, 1H), 7.64 (t, 1H), 7.11 (s, 1H), 6.93 (d, 1H), 4.13 (s, 3H).

### Step 3: Synthesis of 4-bromo-5-methoxy-2-(trifluoromethyl)quinoline

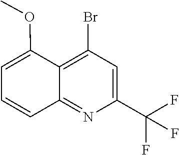 [SMILES: COc1cccc2nc(C(F)(F)F)cc(Br)c12]

To 5-methoxy-2-(trifluoromethyl)quinolin-4-ol (1.5 g, 6.2 mmol) added phosphorus oxybromide (1.8 g, 6.2 mmol). This was heated to 150° C. for 3 h. The reaction mixture was allowed to cool to room temperature and poured into saturated sodium bicarbonate solution, extracted in ethyl acetate (3×200 mL), washed with brine solution (100 mL), dried over anhydrous sodium sulfate, filtered and concentrated under reduced pressure. The residue was purified by (200-400) silica using the ethyl acetate and cyclohexane (10:90), obtained 4-bromo-5-methoxy-2-(trifluoromethyl)quinoline (0.89 g, 47%) as light yellow solid.

¹H NMR (400 MHz, CHLOROFORM-d) δ ppm 7.97 (s, 1H), 7.83 (dd, 1H), 7.73 (t, 1H), 7.06 (d, 1H), 4.00 (s, 3H)

### Step 4: Synthesis of 5-methoxy-4-(4,4,4-trifluorobutyl)-2-(trifluoromethyl) quinoline

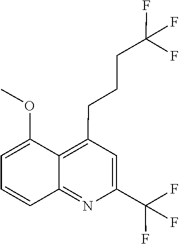 [SMILES: COc1cccc2nc(C(F)(F)F)cc(CCCC(F)(F)F)c12]

To stirred magnesium turnings (0.51 g, 21 mmol) in tetrahydrofuran (10 mL) was added a crystal of iodine. The reaction was stirred for 10 mins and heated to reflux. Then added a small amount of 1-bromo-4,4,4-trifluorobutane in tetrahydrofuran was added. The heating was continued until the solution went from yellow to grey in colour. The remaining 1-bromo-4,4,4-trifluorobutane (2.0 g, 10.00 mmol) solution in tetrahydrofuran (15 mL) was added dropwise under heating condition and then the reaction heated at 75° C. for 1 h, then allowed to cool to RT. The above formed solution of bromo(4,4,4-trifluorobutyl)magnesium in tetrahydrofuran was added dropwise to ice cooled solution of 4-bromo-5-methoxy-2-(trifluoromethyl)quinazoline (0.89 g, 2.91 mmol) and Copper(I) iodide (0.11 g, 0.058 mmol) in tetrahydrofuran (7 mL) at 0° C. and the reaction was stirred at room temperature for 16 hours. The reaction was quenched with saturated aqueous ammonium chloride and extracted with ethyl acetate (3×200 mL), the combined organics were washed with brine (50 mL), dried over sodium sulfate and evaporated to dryness under reduced pressure to give 5-methoxy-4-(4,4,4-trifluorobutyl)-2-(trifluoromethyl)quinazoline (0.275 g, 28.04%).

¹H NMR (400 MHz, CHLOROFORM-d) δ ppm 7.83 (d, 1H), 7.69 (t, 1H), 7.44 (s, 1H), 7.01 (d, 1H), 3.99-4.04 (m, 3H), 3.36-3.43 (m, 2H), 2.14-2.30 (m, 2H), 1.90-2.10 (d, 2H).

### Step 5: Synthesis of 4-(4,4,4-trifluorobutyl)-2-(trifluoromethyl)quinolin-5-ol

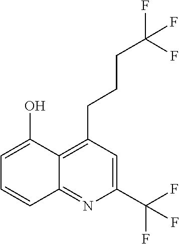 [SMILES: Oc1cccc2nc(C(F)(F)F)cc(CCCC(F)(F)F)c12]

A mixture of 5-methoxy-4-(4,4,4-trifluorobutyl)-2-(trifluoromethyl)quinoline (0.27 g, 0.80 mmol) and pyridin-1-ium;hydrochloride (2.38 g, 20.01 mmol) was s heated at 180° C. for 2 h. The reaction mixture cooled to room temperature, quenched with ice cold water and then with 2N HCl, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (15:85), obtained 4-(4,4,4-trifluorobutyl)-2-(trifluoromethyl)quinolin-5-ol (0.07 g, 25.97%) as light brown solid.

¹H NMR (400 MHz, METHANOL-d4) δ ppm 7.61-7.67 (m, 2H), 7.54 (s, 1H), 7.05 (dd, 1H), 3.47-3.52 (m, 2H), 2.21-2.36 (m, 2H), 1.95-2.05 (m, 2H).

### Step 6: Synthesis of 5-(5-chloropyrimidin-2-yl)oxy-4-(4,4,4-trifluorobutyl)-2-(trifluoromethyl)quinoline (1.015)

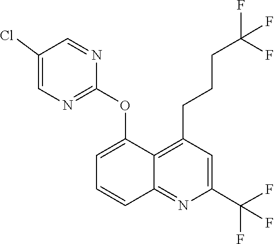 [SMILES: FC(F)(F)CCCc1cc(C(F)(F)F)nc2cccc(Oc3ncc(Cl)cn3)c12]

To a solution of 4-(4,4,4-trifluorobutyl)-2-(trifluoromethyl)quinolin-5-ol (0.054 g, 0.167 mmol) in N,N-dimethylformamide (1.08 mL) was added potassium carbonate (0.035 g, 0.25 mmol) and 5-chloro-2-methylsulfonyl-pyrimidine (0.032 g, 0.167 mmol). This was heated to 55° C. for 1 h. The reaction mixture cooled to room temperature, quenched with ice cold water and, extracted in ethyl acetate (3×50 mL), washed with brine solution (20 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-4-(4,4,4-trifluorobutyl)-2-(trifluoromethyl)quinoline (0.05 g, 68.67%) as light yellow gummy mass.

¹H NMR (400 MHz, CHLOROFORM-d) δ ppm 8.52 (s, 2H), 8.21 (d, 1H), 7.82 (t, 1H), 7.53 (s, 1H), 7.42 (dd, 1H), 3.18-3.25 (m, 2H), 2.14-2.27 (m, 2H), 1.87-1.98 (m, 2H)

### EXAMPLE 15: SYNTHESIS OF 5-(5-CHLOROPYRIMIDIN-2-YL)OXY-4-(4,4,4-TRIFLUOROBUTYL)-2-(TRIFLUOROMETHYL)QUINOLINE-3-CARBONITRILE (1.045)

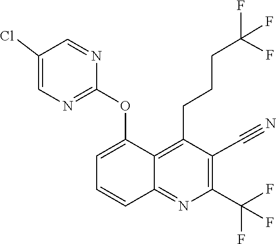 [SMILES: N#Cc1c(C(F)(F)F)nc2cccc(Oc3ncc(Cl)cn3)c2c1CCCC(F)(F)F]

### Step 1: Synthesis of ethyl 5-fluoro-4-(4,4,4-trifluorobutyl)-2-(trifluoromethyl)quinoline-3-carboxylate

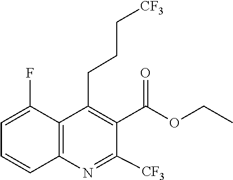 [SMILES: CCOC(=O)c1c(C(F)(F)F)nc2cccc(F)c2c1CCCC(F)(F)F]

To a solution of 1-(2-amino-6-fluoro-phenyl)-5,5,5-trifluoro-pentan-1-one (3.0 g, 12.0 mmol) in N,N-dimethylformamide (15.0 mL) added Copper(I) oxide (0.17 g, 1.2 mmol), ethyl-4,4,4-trifluorobut-2-yoate (2.2 g, 2.2 mmol) and N,N-Diisopropylethylamine (0.21 mL, 1.20 mmol). This was heated to 120° C. for 18 h. The reaction mixture cooled to room temperature, diluted with ice cold water (100 mL) and, extracted in ethyl acetate (3×200 mL), washed with brine solution (100 mL), dried over anhydrous sodium sulfate, filtered and concentrated under reduced pressure. The residue was purified by (200-400) silica using the ethyl acetate and cyclohexane (25:75), obtained ethyl 5-fluoro-4-(4,4,4-trifluorobutyl)-2-(trifluoromethyl)quinoline-3-carboxylate (2.7 g, 55%) as light yellow oil.

¹H NMR (400 MHz, CHLOROFORM-d) δ ppm 8.08 (d, 1H), 7.79 (td, 1H), 7.36-7.48 (m, 1H), 4.49 (q, 2H), 3.23 (td, 2H), 2.21-2.33 (m, 2H), 1.98-2.06 (m, 2H), 1.42 (t, 3H).

### Step 2: Synthesis of ethyl 5-methoxy-4-(4,4,4-trifluorobutyl)-2-(trifluoromethyl) quinoline-3-carboxylate

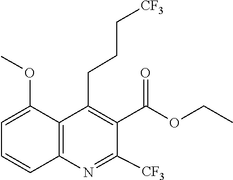 [SMILES: CCOC(=O)c1c(C(F)(F)F)nc2cccc(OC)c2c1CCCC(F)(F)F]

To a solution of ethyl 5-fluoro-4-(4,4,4-trifluorobutyl)-2-(trifluoromethyl)quinoline-3-carboxylate (2.7 g, 6.8 mmol) in Methanol (14 mL) and N,N-Dimethylformamide (14 mL) added sodium methylate (4.7 mL, 20 mmol). This was heated to 90° C. for 18 h. The reaction mixture cooled to room temperature, diluted with ice cold water (100 mL) and extracted in ethyl acetate (3×200 mL), washed with brine solution (100 mL), dried over anhydrous sodium sulfate, filtered and concentrated under reduced pressure. The residue was purified by (200-400) silica using the ethyl acetate and cyclohexane (25:75), obtained ethyl 5-methoxy-4-(4,4,4-trifluorobutyl)-2-(trifluoromethyl)quinoline-3-carboxylate (2.4 g, 86%) as yellow oil.

¹H NMR (400 MHz, CHLOROFORM-d) δ ppm 7.80-7.83 (m, 1H), 7.73 (t, 1H), 7.06 (d, 1H), 4.47 (q, 2H), 4.01 (s, 3H), 3.26-3.39 (m, 2H), 2.23 (m, 3H), 2.00 (m, 2H), 1.41 (t, 2H)

### Step 3: Synthesis of obtained 5-methoxy-4-(4,4,4-trifluorobutyl)-2-(trifluoromethyl)quinoline-3-carboxylic acid

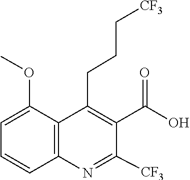 [SMILES: COc1cccc2nc(C(F)(F)F)c(C(=O)O)c(CCCC(F)(F)F)c12]

A mixture of ethyl 5-methoxy-4-(4,4,4-trifluorobutyl)-2-(trifluoromethyl)quinoline-3-carboxylate (1.8 g, 4.4 mmol) and pyridin-1-ium;hydrochloride (10 g, 88 mmol) was heated at 200° C. for 2 h. The reaction mixture cooled to room temperature, quenched with ice cold water and then with 2N HCl, extracted in ethyl acetate (4×200 mL), washed with brine solution (100 mL), dried over anhydrous Na₂SO₄, filtered and concentrated under reduced pressure. The residue was purified by (200-400) silica using the ethyl acetate and cyclohexane (30:70), obtained 5-methoxy-4-(4,4,4-trifluorobutyl)-2-(trifluoromethyl)quinoline-3-carboxylic acid (0.93 g, 53%) as light yellow solid.

¹H NMR (400 MHz, DMSO-d6) δ ppm 7.86 (t, 1H), 7.74 (d, 1H), 7.33 (d, 1H), 4.03 (s, 3H), 3.22-3.46 (m, 2H),), 2.35-2.48 (m, 2H), 1.80-1.92 (m, 2H) one COOH proton missing

### Step 4: Synthesis of 5-methoxy-4-(4,4,4-trifluorobutyl)-2-(trifluoromethyl) quinoline-3-carboxamide

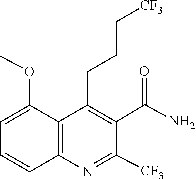 [SMILES: COc1cccc2nc(C(F)(F)F)c(C(N)=O)c(CCCC(F)(F)F)c12]

To a stirred solution of 5-methoxy-4-(4,4,4-trifluorobutyl)-2-(trifluoromethyl)quinoline-3-carboxylic acid (0.4 g, 1.05 mmol) in thionyl chloride (8.0 ml, 109 mmol) was added catalytic N,N-dimethyl formamide. The reaction mass was heated at 60° C. for 60 min. The crude mass obtained after concentration was dissolved in tetrahydrofuran (4 mL) and added dropwise to a stirred solution of aqueous ammonia (13 mL). The reaction mass stirred at room temperature for 16 h. The reaction mixture extracted in ethyl acetate (3×100 mL), washed with brine solution (50 mL), dried over anhydrous sodium sulfate, filtered, and concentrated under reduced pressure to give 5-methoxy-4-(4,4,4-trifluorobutyl)-2-(trifluoromethyl)quinoline-3-carboxamide (0.28 g, 70.23%) as brown gummy mass.

LCMS: RT: 1.09 min; 381.2 (M+H)

### Step 5: Synthesis of 5-methoxy-4-(4,4,4-trifluorobutyl)-2-(trifluoromethyl) quinoline-3-carbonitrile

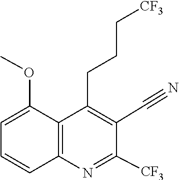 [SMILES: COc1cccc2nc(C(F)(F)F)c(C#N)c(CCCC(F)(F)F)c12]

To a solution of 5-methoxy-4-(4,4,4-trifluorobutyl)-2-(trifluoromethyl)quinoline-3-carboxamide (0.26 g, 0.684 mmol) in dichloromethane (2.6 mL) added triethylamine (0.422 mL, 3.01 mmol) at 0° C., followed by trifluoroacetic anhydride (0.25 mL, 1.71 mmol). The reaction mass stirred at room temperature for 16 h. The reaction mixture diluted with ice cold water and, extracted in ethyl acetate (3×100 mL), washed with brine solution (50 mL), dried over anhydrous sodium sulfate, filtered, and concentrated under reduced pressure. The residue was purified by (200-400) silica using the ethyl acetate and cyclohexane (5:95), to give 5-methoxy-4-(4,4,4-trifluorobutyl)-2-(trifluoromethyl)quinoline-3-carbonitrile (0.112 g, 45.22%) as yellow oil.

¹H NMR (400 MHz, CHLOROFORM-d) δ ppm 7.84-7.91 (m, 2H), 7.16 (dd, 1H), 4.07 (s, 3H), 3.70-3.78 (m, 2H), 2.30-2.42 (m, 2H), 1.98-2.07 (m, 2H).

### Step 6: Synthesis of 5-hydroxy-4-(4,4,4-trifluorobutyl)-2-(trifluoromethyl)quinoline-3-carbonitrile

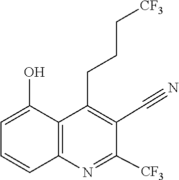 [SMILES: N#Cc1c(C(F)(F)F)nc2cccc(O)c2c1CCCC(F)(F)F]

To a solution of 5-methoxy-4-(4,4,4-trifluorobutyl)-2-(trifluoromethyl)quinoline-3-carbonitrile (0.1 g, 0.2760 mmol) in N-Methyl pyrrolidinone (1.5 mL) added Lithium chloride (0.1170 g, 2.760 mmol) and p-toluene sulphonic acid (0.480 g, 2.76 mmol).

This was heated at 160° C. for 2 h. The reaction mixture cooled to room temperature, quenched with ice cold water and then with 2N hydrochloric acid, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (25:75), to give 5-hydroxy-4-(4,4,4-trifluorobutyl)-2-(trifluoromethyl)quinoline-3-carbonitrile (0.065 g, 64.93%) as thick gummy mass.

¹H NMR (400 MHz, METHANOL-d4) δ ppm 7.81 (t, 1H), 7.69 (dd, 1H), 7.19 (dd, 1H), 3.74-3.81 (m, 2H), 2.30-2.47 (m, 2H), 1.98-2.07 (m, 2H).

### Step 7: Synthesis of 5-(5-chloropyrimidin-2-yl)oxy-4-(4,4,4-trifluorobutyl)-2-(trifluoromethyl)quinoline-3-carbonitrile (1.045)

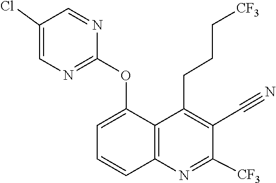 [SMILES: N#Cc1c(C(F)(F)F)nc2cccc(Oc3ncc(Cl)cn3)c2c1CCCC(F)(F)F]

To a solution of 5-hydroxy-4-(4,4,4-trifluorobutyl)-2-(trifluoromethyl)quinoline-3-carbonitrile (0.05 g, 0.144 mmol) in isopropyl alcohol (1.00 mL) added Potassium carbonate (0.030 g, 0.215 mmol) and 5-chloro-2-methylsulfonyl-pyrimidine (0.042 g, 0.215 mmol). This was heated to 50° C. for 16 h. The reaction mixture cooled to room temperature, quenched with ice cold water and, extracted in ethyl acetate (3×50 mL), washed with brine solution (20 mL), dried over anhydrous sodium sulfate, filtered and concentrated under reduced pressure. The residue was purified by (200-400) silica using the ethyl acetate and cyclohexane (10:90), obtained 5-(5-chloropyrimidin-2-yl)oxy-4-(4,4,4-trifluorobutyl)-2-(trifluoromethyl)quinoline-3-carbonitrile (0.029 g, 43.83%) as off white solid.

¹H NMR: 400 MHz, CHLOROFORM-d) δ ppm 8.55 (s, 2H), 8.24 (dd, 1H), 7.99 (t, 1H), 7.57 (dd, 1H), 3.56-3.64 (m, 2H), 2.24-2.38 (m, 2H), 1.87-1.97 (m, 2H).

### EXAMPLE 16: SYNTHESIS 8-(5-CHLOROPYRIMIDIN-2-YL)OXY-1-(4,4,4-TRIFLUOROBUTYL)ISOQUINOLINE (1.012)

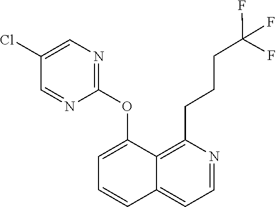 [SMILES: FC(F)(F)CCCc1nccc2cccc(Oc3ncc(Cl)cn3)c12]

### Step 1: Synthesis of 2-benzoyl-8-methoxy-1H-isoquinoline-1-carbonitrile

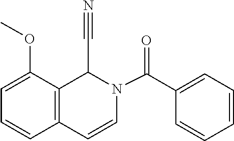 [SMILES: COc1cccc2c1C(C#N)N(C(=O)c1ccccc1)C=C2]

To a solution of 8-methoxyisoquinoline (1 g, 6.28 mmol) in dichloromethane (20 mL) was added anhydrous Aluminium chloride (0.0042 g, 0.0314 mmol), Trimethysilyl cyanide (1.71 mL, 12.564 mmol) dropwise followed by benzoyl chloride (1.62 mL, 13.821 mmol). The reaction mixture was warmed at 30° C. The reaction mass was stirred at room temperature for 8 h. The reaction mass was quenched with water (100 mL), stirred for 30 min and the organic layer was separated. The organic layer was washed with 1N hydrochloric (100 mL), water (100 mL), 1N sodium hydroxide (100 mL) and finally water (100 mL). The organic layer was dried over anhydrous sodium sulfate, filtered and concentrated to give gummy mass which was then triturated with diethyl ether (30 mL×2) to give 2-benzoyl-8-methoxy-1H-isoquinoline-1-carbonitrile (1.17 g, 64.15%) as brown solid.

¹H NMR (400 MHz, CHLOROFORM-d) δ ppm 8.08-8.18 (m, 1H), 7.63-7.73 (m, 1H), 7.44-7.61 (m, 4H), 7.34 (t, 1H), 6.87 (d, 1H), 6.80 (d, 1H), 6.53-6.67 (m, 1H), 5.98 (br d, 1H).

### Step 2: Synthesis of 8-methoxy-1-(4,4,4-trifluorobutyl)isoquinoline

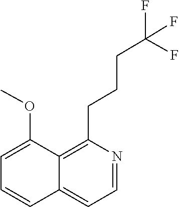 [SMILES: COc1cccc2ccnc(CCCC(F)(F)F)c12]

To a suspension of sodium hydride (0.20 g, 4.1 mmol) in N,N-Dimethylformamide (17 mL) was added dropwise a solution of 2-benzoyl-8-methoxy-1H-isoquinoline-1-carbonitrile (1.0 g, 3.4 mmol) and 1-bromo-4,4,4-trifluorobutane (0.79 g, 4.1 mmol) in N,N-Dimethylformamide (17 mL) at −10° C. After complete addition, the reaction mass was allowed to come to room temperature and stirred for 16 h. The reaction mixture was quenched with saturated ammonium chloride solution, extracted in ethyl acetate (3×200 mL), washed with brine solution (10 mL), dried over anhydrous sodium sulfate, filtered and concentrated under reduced pressure. The residue was purified by (200-400) silica using the ethyl acetate and cyclohexane (10:90) to give 8-methoxy-1-(4,4,4-trifluorobutyl)isoquinoline (0.33 g, 36%) as light yellow solid.

¹H NMR (400 MHz, CHLOROFORM-d) δ ppm 8.37 (d, 1H), 7.55 (t, 1H), 7.44 (d, 1H), 7.36 (d, 1H), 6.92 (d, 1H), 3.99 (s, 3H), 3.49-3.57 (m, 2H), 2.18-2.30 (m, 2H), 2.00-2.13 (m, 2H).

### Step 3: Synthesis of 1-(4,4,4-trifluorobutyl)isoquinolin-8-ol

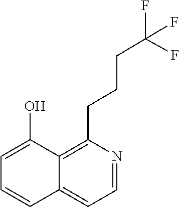 [SMILES: Oc1cccc2ccnc(CCCC(F)(F)F)c12]

A mixture of 8-methoxy-1-(4,4,4-trifluorobutyl)isoquinoline (0.27 g, 1.00 mmol) and pyridin-1-ium;hydrochloride (2.98 g, 25.06 mmol) was heated at 210° C. for 2 h. The reaction mixture cooled to room temperature, quenched with ice cold water and then with 2N HCl, extracted in ethyl acetate (3×50 mL), washed with brine solution (50 mL), dried over anhydrous sodium sulfate, filtered and concentrated under reduced pressure. The residue was purified by (200-400) silica using the ethyl acetate and cyclohexane (20:80) to give 1-(4,4,4-trifluorobutyl)isoquinolin-8-ol (0.144 g, 54.03%) as thick gummy mass.

¹H NMR (400 MHz, METHANOL-d4) δ ppm 8.18 (d, 1H), 7.51-7.59 (m, 2H), 7.35 (d, 1H), 7.00 (d, 1H), 3.53-3.59 (m, 2H), 2.18-2.33 (m, 2H), 1.98-2.06 (m, 2H)

### Step 4: Synthesis of 8-(5-chloropyrimidin-2-yl)oxy-1-(4,4,4-trifluorobutyl)isoquinoline (1.012)

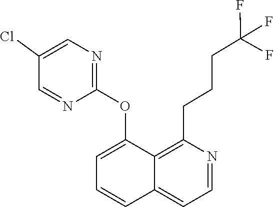 [SMILES: FC(F)(F)CCCc1nccc2cccc(Oc3ncc(Cl)cn3)c12]

To a solution of 1-(4,4,4-trifluorobutyl)isoquinolin-8-ol (0.16 g, 0.627 mmol) in N,N-Dimethylformamide (3.2 mL) added Potassium carbonate (0.268 g, 1.88 mmol) and 5-chloro-2-methylsulfonyl-pyrimidine (0.145 g, 0.752 mmol). This was heated to 60° C. for 2 h. The reaction mixture cooled to room temperature, quenched with ice cold water and extracted in ethyl acetate (3×50 mL), washed with brine solution (20 mL), dried over anhydrous sodium sulfate, filtered and concentrated under reduced pressure. The residue was purified by (200-400) silica using the ethyl acetate and cyclohexane (10:90), obtained 8-(5-chloropyrimidin-2-yl)oxy-1-(4,4,4-trifluorobutyl)isoquinoline (0.148 g, 64.18%) as light brown gummy mass.

¹H NMR (400 MHz, CHLOROFORM-d) δ ppm 8.52 (s, 2H), 8.45 (d, 1H), 7.77 (dd, 1H) 7.68 (t, 1H), 7.58 (d, 1H), 7.30 (dd, 1H), 3.33-3.39 (m, 2H) 2.11-2.23 (m, 2H) 1.96-2.06 (m, 2H).

### EXAMPLE 17: SYNTHESIS OF 3-CHLORO-5-(5-CHLOROPYRIMIDIN-2-YL) OXY-2-METHYL-4-(4,4,4-TRIFLUOROBUTYL) QUINOLINE (1.040)

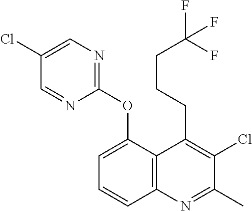 [SMILES: Cc1nc2cccc(Oc3ncc(Cl)cn3)c2c(CCCC(F)(F)F)c1Cl]

### Step 1: Synthesis of 3-chloro-5-fluoro-2-methyl-4-(4,4,4-trifluorobutyl) quinoline

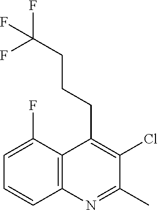 [SMILES: Cc1nc2cccc(F)c2c(CCCC(F)(F)F)c1Cl]

To a solution of 1-(2-amino-6-fluoro-phenyl)-5,5,5-trifluoro-pentan-1-one (400 mg, 1.6051 mmol) in N,N-dimethylformamide (2 mL) added 1-chloropropan-2-one (0.1782 g, 1.9262 mmol) at 0° C. followed by choro(trimethyl)silane (1.0 mL, 8.0257 mmol) dropwise. Then warmed to room temperature overnight, heated under MW irradiation at 100° C. for 2 h. Quenched with water (10 mL), extracted in ethyl acetate (3×10 mL), dried over anhydrous (Na₂SO₄), filtered and concentrated under reduced pressure. The residue was purified by (200-400) silica using the ethyl acetate and cyclohexane (30:70), obtained white solid (220 mg, 44%)

¹H NMR (400 MHz, CHLOROFORM-d) δ ppm 7.89 (d, 1H) 7.58-7.63 (m, 1H) 7.20-7.25 (m, 1H) 3.45 (td, 2H) 2.85 (s, 3H) 2.23-2.36 (m, 2H) 1.90-2.02 (m, 2H)

### Step 2: Synthesis of 3-chloro-5-methoxy-2-methyl-4-(4,4,4-trifluorobutyl) quinoline

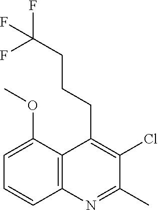 [SMILES: COc1cccc2nc(C)c(Cl)c(CCCC(F)(F)F)c12]

To a solution of 3-chloro-5-fluoro-2-methyl-4-(4,4,4-trifluorobutyl) quinoline (220 mg, 0.7197 mmol) in methanol (2.2 mL) and DMF (2.2 mL) added sodium methoxide (2.159 mmol, 25 mass %, 0.4753 g,) then heated to 60° C. for 60 h. The reaction mixture was cooled to room temperature, quenched with aqueous solution of ammonium chloride (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80), obtained yellow gummy mass (180 mg, 86%).

¹H NMR (400 MHz, CHLOROFORM-d) δ ppm 7.48-7.63 (m, 2H), 6.89 (dd, 1H), 3.97 (s, 3H), 3.50-3.64 (m, 2H), 2.79 (s, 3H), 2.21-2.37 (m, 2H), 1.89-2.00 (m, 2H)

### Step 3: Synthesis of 3-chloro-2-methyl-4-(4,4,4-trifluorobutyl) quinolin-5-ol

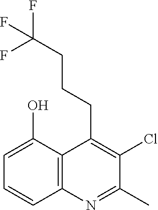 [SMILES: Cc1nc2cccc(O)c2c(CCCC(F)(F)F)c1Cl]

A mixture of 3-chloro-5-methoxy-2-methyl-4-(4,4,4-trifluorobutyl) quinoline (180 mg, 0.3399 mmol) and pyridine hydrochloride (0.9 mL, 115.562) was heated to 180° C. for 2 h. The reaction mixture was cooled to room temperature, quenched with aqueous solution of ammonium chloride (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80) to give yellow gummy mass (30 mg, 29%).

¹H NMR (400 MHz, CHLOROFORM-d) δ ppm 7.61 (d, 1H), 7.45 (t, 1H), 6.79 (d, 1H), 3.56-3.65 (m, 2H), 2.79 (s, 3H), 2.23-2.34 (m, 2H), 1.96-2.04 (m, 2H)

### Step 4: Synthesis of 3-chloro-5-(5-chloropyrimidin-2-yl) oxy-2-methyl-4-(4,4,4-trifluorobutyl) quinoline (1.040)

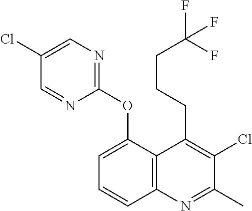 [SMILES: Cc1nc2cccc(Oc3ncc(Cl)cn3)c2c(CCCC(F)(F)F)c1Cl]

To a solution of 3-chloro-2-methyl-4-(4,4,4-trifluorobutyl) quinolin-5-ol (30 mg, 0.09878 mmol) in dimethyl formamide (1 mL) was added Potassium Carbonate (41.37 mg, 0.2963 mmol) followed by 5-methyl-2-methylsulfonyl-pyrimidine (17 mg, 0.09878 mmol) then heated to 50° C. for 2 h. The reaction mixture was cooled to room temperature, extracted in ethyl acetate (3×10 mL) washed with brine (200 mL), dried over anhydrous sodium sulfate, filtered and concentrated under reduced pressure. The residue was purified by (200-400) silica using the ethyl acetate and cyclohexane (20:80) to give brown solid (16 mg, 38.92%).

1H NMR (400 MHz, CHLOROFORM-d) δ ppm 8.51 (s, 2H), 7.97 (dd, 1H), 7.67 (dd, 1H), 7.21-7.25 (m, 1H), 3.32-3.44 (m, 2H), 2.82 (s, 3H), 2.12-2.29 (m, 2H), 1.70-1.83 (m, 2H)

### EXAMPLE 18: 5-(5-CHLOROPYRIMIDIN-2-YL) OXY-2-METHYL-4-(4,4,4-TRIFLUOROBUTYL) QUINAZOLINE-7-CARBONITRILE (1.018)

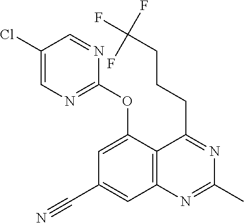 [SMILES: Cc1nc(CCCC(F)(F)F)c2c(Oc3ncc(Cl)cn3)cc(C#N)cc2n1]

### Step 1: Synthesis of 1-(4-bromo-2,6-difluoro-phenyl)-5,5,5-trifluoro-pentan-1-ol

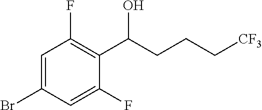 [SMILES: OC(CCCC(F)(F)F)c1c(F)cc(Br)cc1F]

To stirred magnesium turnings (1.1 g, 45 mmol) in THF (30 mL) was added a crystal of iodine. The reaction was stirred for 10 mins and heated to reflux. Then added a small amount of 1-bromo-4,4,4-trifluorobutane in THF was added. The heating was continued until the solution went from yellow to grey in colour. The remaining 1-bromo-4,4,4-trifluorobutane (7.3 g, 38 mmol) solution in THF (30 ml) was added dropwise under heating condition and then the reaction heated at 75° C. for 1 h, then allowed to cool to RT. The above formed solution of bromo(4,4,4-trifluorobutyl) magnesium in THF was added dropwise to a cooled solution of 4-bromo-2,6-difluoro-benzaldehyde (5 g, 23 mmol) in THF (40 mL) at 0° C. and the reaction was stirred at room temperature for 16 h. The reaction was quenched with saturated aqueous ammonium chloride (100 mL) and extracted with ethyl acetate (3×200 mL). The combined organics were washed with brine (100 mL), dried over anhydrous sodium sulfate and evaporated to dryness under reduced pressure to give 1-(4-bromo-2,6-difluoro-phenyl)-5,5,5-trifluoro-pentan-1-ol (7.4 g, 96%) as yellow oil.

1H NMR (400 MHz, CHLOROFORM-d) δ ppm 7.01-7.19 (m, 2H) 5.00 (dd, 1H) 2.06-2.21 (m, 2H), 1.71-1.95 (m, 2H) 1.49-1.69 (m, 2H) one hydroxy missing

### Step 2: Synthesis of 1-(4-bromo-2,6-difluoro-phenyl)-5,5,5-trifluoro-pentan-1-one

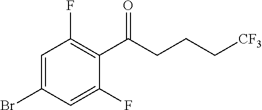 [SMILES: O=C(CCCC(F)(F)F)c1c(F)cc(Br)cc1F]

To a solution of 1-(4-bromo-2,6-difluoro-phenyl)-5,5,5-trifluoro-pentan-1-ol (7.5 g, 22 mmol) in acetonitrile (150 mL) was added (1,1-diacetoxy-3-oxo-1lambda5,2-benziodoxol-1-yl) acetate (10 g, 23 mmol) at 0° C. portion wise over a period of 15 mins. The resultant mixture was stirred at room temperature overnight. Then quenched with solution of sodium thiosulfate (11 g, 66 mmol) in water (100 mL). It was stirred for 10 min, then added the solution of sodium hydrogen carbonate to adjust the basic pH and extracted in ethyl acetate (3×100 mL) washed with brine (200 mL), dried over anhydrous (Na₂SO₄), filtered and concentrated under reduced pressure. The residue was purified by (200-400) silica using the ethyl acetate and cyclohexane (10:90), afforded yellow liquid (7.5 g, quant.)

¹H NMR (400 MHz, CHLOROFORM-d) δ ppm 7.16 (d, 2H), 2.94 (t, 2H), 2.09-2.27 (m, 2H), 1.96-2.02 (m, 2H)

### Step 3: Synthesis of 1-(2-amino-4-bromo-6-fluoro-phenyl)-5,5,5-trifluoro-pentan-1-one

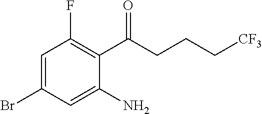 [SMILES: Nc1cc(Br)cc(F)c1C(=O)CCCC(F)(F)F]

To a solution of 1-(4-bromo-2,6-difluoro-phenyl)-5,5,5-trifluoro-pentan-1-one (7.5 g, 22 mmol) in Acetonitrile (30 mL) was added ammonium hydroxide (88 mL, 660 mmol) then resultant mixture was heated at 120° C. in Eyela equipment for 10 h. The reaction mixture was diluted with 50 mL water and extracted in ethyl acetate (3×100 mL) washed with brine (100 mL), dried over anhydrous sodium sulfate, filtered and concentrated under reduced pressure. The residue was purified by (200-400) silica using the ethyl acetate and cyclohexane (10:90), afforded yellow liquid (4 g, 48%.)

¹H NMR (400 MHz, CHLOROFORM-d) δ ppm 6.53 (d, 2H) 6.30-6.50 (br s, 2H) 2.95-3.04 (m, 2H) 2.09-2.23 (m, 2H) 1.93-1.97 (m, 2H)

### Step 4: Synthesis of 7-bromo-5-fluoro-2-methyl-4-(4,4,4-trifluorobutyl) quinazoline

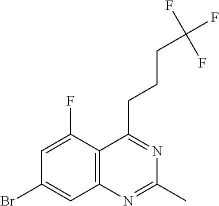 [SMILES: Cc1nc(CCCC(F)(F)F)c2c(F)cc(Br)cc2n1]

To a suspension of 1-(2-amino-4-bromo-6-fluoro-phenyl)-5,5,5-trifluoro-pentan-1-one (3.5 g, 9.6 mmol), in 2,4-pentanedione (1.2 mL, 12 mmol) was added ammonium acetate (2.2 g, 29 mmol,) stirred the reaction mixture at 130° C. for 3 h. Then quenched in water (50 mL), extracted in ethyl acetate (3×50 mL), dried over anhydrous sodium sulfate, filtered and concentrated under reduced pressure. The residue was purified by (200-400) silica using the ethyl acetate and cyclohexane (25:75) to give yellow liquid (1.5 g, 44%)

¹H NMR (400 MHz, CHLOROFORM-d) δ ppm 7.94-7.96 (m, 1H), 7.36-7.39 (m, 1H), 3.35-3.70 (m, 2H), 2.83 (s, 3H), 2.20-2.35 (m, 2H), 2.06-2.20 (m, 2H)

### Step 5: Synthesis of 7-bromo-5-methoxy-2-methyl-4-(4,4,4 trifluoro butyl) quinazoline

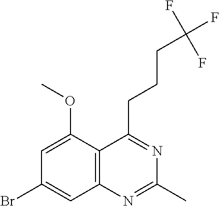 [SMILES: COc1cc(Br)cc2nc(C)nc(CCCC(F)(F)F)c12]

To a solution of 7-bromo-5-fluoro-2-methyl-4-(4,4,4-trifluorobutyl) quinazoline (1.4 g, 4.0 mmol) in Methanol (11 mL) was added Sodium methylate (2.7 mL, 12 mmol, 25 mass %) at 0° C. then warmed to room temperature overnight. quenched with ice cold water and filtered the precipitate as a yellow solid (1.1 gm, 76%).

¹H NMR (400 MHz, CHLOROFORM-d) δ ppm 7.71 (d, 1H), 6.99 (d, 1H), 4.01 (s, 3H), 3.38-3.46 (m, 2H), 2.79 (s, 3H), 2.22-2.29 (m, 2H), 1.98-2.11 (m, 2H)

### Step 6: Synthesis of 7-bromo-2-methyl-4-(4,4,4-trifluorobutyl) quinazolin-5-ol

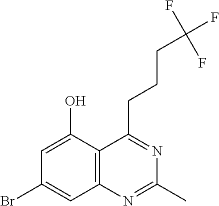 [SMILES: Cc1nc(CCCC(F)(F)F)c2c(O)cc(Br)cc2n1]

A solution of 7-bromo-5-methoxy-2-methyl-4-(4,4,4-trifluorobutyl) quinazoline (1.1 g, 3.0 mmol) in hydrobromic acid (48 mass %) in water (5.5 mL) was heated at 130° C. for 5 h. The reaction mixture was diluted with water (20 mL), extracted in ethyl acetate (3×50 mL), dried over anhydrous sodium sulfate, filtered and concentrated under reduced pressure. The residue was purified by (200-400) silica using the ethyl acetate and cyclohexane (50:50) to give yellow solid (400 mg, 38%)

¹H NMR (400 MHz, CHLOROFORM-d) δ ppm 11.56 (br s, 1H), 7.48 (d, 1H), 7.07 (d, 1H), 3.45 (t, 2H), 2.65 (s, 3H), 2.31-2.45 (m, 2H), 1.88-2.02 (m, 2H)

### Step 7: Synthesis of 7-bromo-5-(5-chloropyrimidin-2-yl) oxy-2-methyl-4-(4,4,4-trifluorobutyl) quinazoline (1.034)

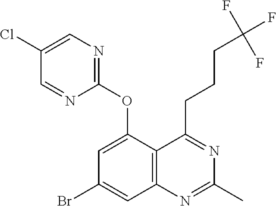 [SMILES: Cc1nc(CCCC(F)(F)F)c2c(Oc3ncc(Cl)cn3)cc(Br)cc2n1]

To a solution of 7-bromo-2-methyl-4-(4,4,4-trifluorobutyl) quinazolin-5-ol (200 mg, 0.5727 mmol) in isopropyl alcohol (4 mL) was added potassium carbonate (0.239 g, 1.718 mmol) and 5-chloro-2-methylsulfonyl-pyrimidine (0.3310 g, 1.718 mmol). The resultant mixture was heated to 50° C. for 16 h. Then quenched in water (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80) to give yellow solid (120 mg, 45.38%)

¹H NMR (400 MHz, CHLOROFORM-d) δ ppm 8.54 (s, 2H), 8.07 (d, 1H), 7.40 (d, 1H), 3.29 (t, 2H), 2.83 (s, 3H), 2.12-2.24 (m, 2H), 1.96-2.05 (m, 2H)

### Step 8: Synthesis of 5-(5-chloropyrimidin-2-yl) oxy-2-methyl-4-(4,4,4-trifluorobutyl) quinazoline-7-carbonitrile (1.018)

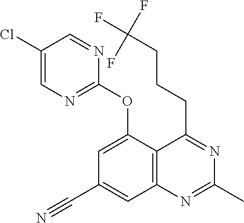 [SMILES: Cc1nc(CCCC(F)(F)F)c2c(Oc3ncc(Cl)cn3)cc(C#N)cc2n1]

To a solution of 7-bromo-5-(5-chloropyrimidin-2-yl) oxy-2-methyl-4-(4,4,4-trifluorobutyl) quinazoline (50 mg, 0.1083 mmol,) in N, N-dimethylformamide (1 mL) added 4,5-bis(diphenylphosphino)-9,9-dimethylxanthene (12.92 mg, 0.02166 mmol), Tris(dibenzylideneacetone) dipalladium (0) (10.22 mg, 0.01083 mmol) followed by zinc cyanide (19.08 mg, 0.1624 mmol). The reaction mixture was heated to 90° C. for 4 h. Then quenched in water (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80) to give yellow solid (24 mg, 54.34%)

¹H NMR (400 MHz, CHLOROFORM-d) δ ppm 8.55 (s, 2H), 8.20 (d, 1H), 7.47 (d, 1H), 3.36 (t, 2H), 2.86 (s, 3H), 2.15-2.27 (m, 2H), 1.99-2.08 (m, 2H)

### EXAMPLE 19: SYNTHESIS OF 5-(5-CHLOROPYRIMIDIN-2-YL) OXY-2,3-DIMETHYL-4-(4,4,4-TRIFLUOROBUTYL) QUINOLINE (1.042)

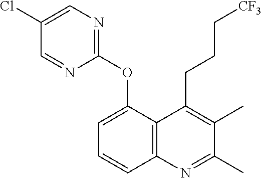 [SMILES: Cc1nc2cccc(Oc3ncc(Cl)cn3)c2c(CCCC(F)(F)F)c1C]

### Step 1: Synthesis of 5-fluoro-2,3-dimethyl-4-(4,4,4-trifluorobutyl) quinoline

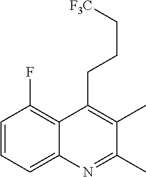 [SMILES: Cc1nc2cccc(F)c2c(CCCC(F)(F)F)c1C]

To a solution of 1-(2-amino-6-fluoro-phenyl)-5,5,5-trifluoro-pentan-1-one (700 mg, 2.80 mmol), in butan-2-one (2.5 mL, 28.09 mmol) was added p-toluene sulfonic acid (489 mg, 2.80 mmol). The resultant mixture was heated under microwave irradiation at 100° C. for 2 h. The reaction mass was then quenched in water (20 mL), extracted in ethyl acetate (3×20 mL), dried over anhydrous sodium sulfate filtered and concentrated under reduced pressure. The residue was purified by (200-400) silica using the ethyl acetate and cyclohexane (20:80) to give yellow solid (380 mg, 47.42%)

### Step 2: Synthesis of 5-methoxy-2,3-dimethyl-4-(4,4,4-trifluorobutyl) quinoline

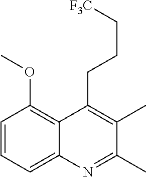 [SMILES: COc1cccc2nc(C)c(C)c(CCCC(F)(F)F)c12]

To a solution of 5-fluoro-2,3-dimethyl-4-(4,4,4-trifluorobutyl) quinoline (0.38 g, 1.332 mmol) in Methanol (3.8 mL) and DMF (1.9 mL) was added Sodium methylate (0.457 mL, 1.998 mmol) at room temperature. After complete addition, it was heated at 80° C. for 12 h. The reaction mass was then quenched with aqueous solution of ammonium chloride (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80) to give off white solid (190 mg, 47.98%).

### Step 3: Synthesis of 2,3-dimethyl-4-(4,4,4-trifluorobutyl) quinolin-5-ol

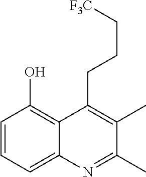 [SMILES: Cc1nc2cccc(O)c2c(CCCC(F)(F)F)c1C]

A suspension of 5-methoxy-2,3-dimethyl-4-(4,4,4-trifluorobutyl) quinoline (0.15 g, 0.5045 mmol) and pyridine hydrochloride (1.500 g, 12.61 mmol) was heated to 180° C. for 3 h. Then quenched in 1N hydrochloric acid (20 mL), extracted in ethyl acetate (3×20 mL), dried over anhydrous sodium sulfate, filtered and concentrated under reduced pressure. The residue was purified by (200-400) silica using the ethyl acetate and cyclohexane (20:80), afforded yellow mass (110 mg, 73.88%)

1H NMR (400 MHz, CHLOROFORM-d) δ ppm 7.42 (br d, 1H) 7.26-7.34 (m, 1H) 6.82 (br d, 1H) 3.38-3.42 (m, 2H) 2.61 (s, 3H) 2.32 (s, 3H) 2.21-2.23 (m, 2H) 1.76-1.94 (m, 2H).

### Step 4: Synthesis of 5-(5-chloropyrimidin-2-yl) oxy-2,3-dimethyl-4-(4,4,4 trifluoro butyl) quinoline (1.042)

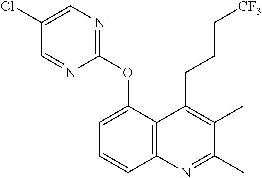 [SMILES: Cc1nc2cccc(Oc3ncc(Cl)cn3)c2c(CCCC(F)(F)F)c1C]

To a solution of 2,3-dimethyl-4-(4,4,4-trifluorobutyl) quinolin-5-ol (0.03 g, 0.1059 mmol) and potassium carbonate (0.03149 g, 0.3177 mmol) in N, N-dimethylformamide (1.059 mL) was added 5-chloro-2-methylsulfonyl-pyrimidine (0.02448 g, 0.1271 mmol) Then reaction mixture was heated at 50° C. for 4 h. Then quenched with aqueous solution of ammonium chloride (10 mL), extracted in ethyl acetate (3×10 mL), dried over anhydrous sodium sulfate, filtered and concentrated under reduced pressure. The residue was purified by (200-400) silica using the ethyl acetate and cyclohexane (20:80), obtained off white solid (7 mg, 16.17%).

¹H NMR (400 MHz, CHLOROFORM-d) δ ppm 8.52 (s, 2H) 7.99 (br d, 1H) 7.62 (t, 1H) 7.14-7.22 (m, 1H) 3.14-3.26 (m, 2H) 2.73 (s, 3H) 2.42 (s, 3H) 2.15-2.29 (m, 2H) 1.69-1.83 (m, 2H)

### EXAMPLE 20: 5-(5-CHLOROPYRIMIDIN-2-YL) OXY-2-METHYL-4-(4,4,4 TRIFLUOROBUTYL) QUINOLINE-3-CARBONITRILE (1.043)

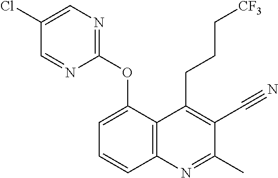 [SMILES: Cc1nc2cccc(Oc3ncc(Cl)cn3)c2c(CCCC(F)(F)F)c1C#N]

### Step 1: Synthesis of 5-fluoro-2-methyl-4-(4,4,4-trifluorobutyl) quinoline-3-carbonitrile

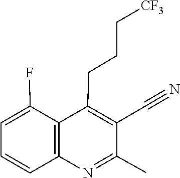 [SMILES: Cc1nc2cccc(F)c2c(CCCC(F)(F)F)c1C#N]

To a solution of 1-(2-amino-6-fluoro-phenyl)-5,5,5-trifluoro-pentan-1-one (200 mg, 0.80257 mmol) in N,N-dimethylformamide (2 mL) was added 3-oxobutanenitrile (0.066 g, 0.80 mmol) at 0° C. followed by choro(trimethyl)silane (0.52 mL, 4.01 mmol) dropwise then stirred the reaction mixture at room temperature overnight. The reaction mixture was slowly added in the crushed ice. The solid precipitated was filtered and dried over high vacuum to obtained yellow solid (200 mg, 84%)

¹H NMR (400 MHz, CHLOROFORM-d) δ ppm 7.89 (d, 1H), 7.76 (td, 1H), 7.27-7.33 (m, 1H), 3.47-3.54 (m, 2H), 2.92 (s, 3H), 2.26-2.39 (m, 2H), 1.98-2.06 (m, 2H)

### Step 2: Synthesis of 5-methoxy-2-methyl-4-(4,4,4-trifluorobutyl) quinoline-3-carbonitrile

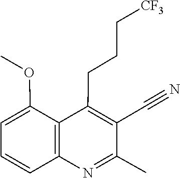 [SMILES: COc1cccc2nc(C)c(C#N)c(CCCC(F)(F)F)c12]

To a solution of 5-fluoro-2-methyl-4-(4,4,4-trifluorobutyl) quinoline-3-carbonitrile (200 mg, 0.6750 mmol) in methanol (2 mL) and dimethyl formamide (2 mL) was added sodium methoxide (0.4457 g, 2.025 mmol, 25 mass %) at room temperature. After complete addition, it was heated at 40° C. for 12 h. The reaction mass was quenched with saturated solution of ammonium chloride (20 mL), extracted in ethyl acetate (3×20 mL), dried over anhydrous sodium sulfate, filtered and concentrated under reduced pressure. The residue was purified by (200-400) silica using the ethyl acetate and cyclohexane (30:70), afforded yellow solid (120 mg, 57%).

LCMS: RT: 1.51 min; 309 (M+H)

### Step 3: Synthesis of 5-hydroxy-2-methyl-4-(4,4,4-trifluorobutyl) quinoline-3-carbonitrile

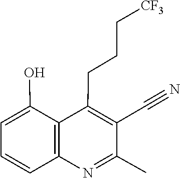 [SMILES: Cc1nc2cccc(O)c2c(CCCC(F)(F)F)c1C#N]

A mixture of 5-methoxy-2-methyl-4-(4,4,4-trifluorobutyl) quinoline-3-carbonitrile (100 mg, 0.32 mmol) and 4-methylbenzenesulfonic acid (0.282 g, 1.62 mmol) in N-methyl-2-pyrrolidone (2 mL) was added lithium chloride (0.068 g, 1.62 mmol), heated the reaction mass at 160° C. for 6 h and extracted in ethyl acetate (3×20 mL). The organic portion was dried over anhydrous sodium sulfate, filtered and concentrated under reduced pressure. The residue was purified by (200-400) silica using the ethyl acetate and cyclohexane (40:60), afforded yellow solid (40 mg, 42%)

¹H NMR (400 MHz, CHLOROFORM-d) δ ppm 7.64-7.72 (m, 1H), 7.58-7.63 (m, 1H), 6.92 (br d, 1H), 3.59-3.75 (m, 2H), 2.91 (s, 3H), 2.02-2.35 (m, 2H), 2.04 (br t, 2H)

### Step 4: Synthesis of 5-(5-chloropyrimidin-2-yl) oxy-2-methyl-4-(4,4,4-trifluorobutyl) quinoline-3-carbonitrile (1.043)

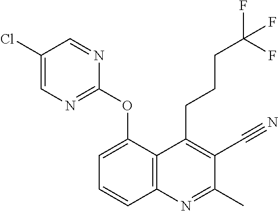 [SMILES: Cc1nc2cccc(Oc3ncc(Cl)cn3)c2c(CCCC(F)(F)F)c1C#N]

To a solution of 5-hydroxy-2-methyl-4-(4,4,4-trifluorobutyl) quinoline-3-carbonitrile (30 mg, 0.10 mmole) in dimethyl formamide (1.5 mL) was added Potassium Carbonate (0.043 g, 0.30 mmol) and 5-chloro-2-methylsulfonyl-pyrimidine (0.019 g, 0.11 mmol) then heated the reaction at 50° C. for 2 h. The reaction mixture was quenched with ice cold water and filtered to give precipitate which was dried over the high vacuum to give yellow solid (26 mg, 62.70%)

¹H NMR (400 MHz, CHLOROFORM-d) δ ppm 8.53 (s, 2H), 8.01 (d, 1H), 7.82 (t, 1H), 7.33 (d, 1H), 3.38-3.53 (m, 2H), 2.91 (s, 3H), 2.19-2.33 (m, 2H), 1.83-1.93 (m, 2H)

### EXAMPLE 21: 5-(5-CHLOROPYRIMIDIN-2-YL) OXY-2-CYCLOPROPYL-4-(4,4,4-TRIFLUOROBUTYL) QUINOLINE-3-CARBONITRILE (1.044)

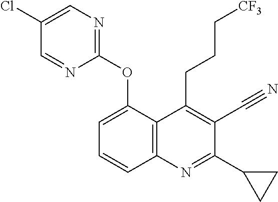 [SMILES: N#Cc1c(C2CC2)nc2cccc(Oc3ncc(Cl)cn3)c2c1CCCC(F)(F)F]

### Step 1: Synthesis of 2-cyclopropyl-5-fluoro-4-(4,4,4-trifluorobutyl) quinoline-3-carbonitrile

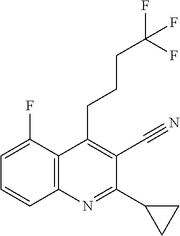 [SMILES: N#Cc1c(C2CC2)nc2cccc(F)c2c1CCCC(F)(F)F]

To a solution of 1-(2-amino-6-fluoro-phenyl)-5,5,5-trifluoro-pentan-1-one (0.5 g, 2.0064 mmol) and 3-cyclopropyl-3-oxo-propanenitrile (0.21896 g, 2.0064 mmol) in N, N-dimethylformamide (5 mL) was added choro(trimethyl)silane (1.3 mL, 10.03 mmol) at 0° C. The reaction mass was stirred at room temperature for 20 h. Then quenched with aqueous solution of ammonium chloride (20 mL), extracted in ethyl acetate (3×20 mL), dried over anhydrous sodium sulfate, filtered and concentrated under reduced pressure. The residue was purified by (200-400) silica using the ethyl acetate and cyclohexane (30:70), afforded yellow solid (400 mg, 61.80%).

¹H NMR (400 MHz, CHLOROFORM-d) δ ppm 7.67-7.80 (m, 1H) 7.57-7.66 (m, 1H) 7.06-7.16 (m, 1H) 3.44 (td, 2H) 2.54-2.74 (m, 1H) 2.20-2.38 (m, 2H) 1.90-2.03 (m, 2H) 1.27-1.31 (m, 2H) 1.12-1.16 (m, 2H)

### Step 2: Synthesis of 2-cyclopropyl-5-hydroxy-4-(4,4,4-trifluorobutyl) quinoline-3-carbonitrile

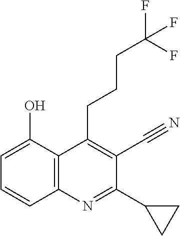 [SMILES: N#Cc1c(C2CC2)nc2cccc(O)c2c1CCCC(F)(F)F]

To a solution of 2-cyclopropyl-5-fluoro-4-(4,4,4-trifluorobutyl) quinoline-3-carbonitrile (0.19 g, 0.5895 mmol) in dimethyl sulfoxide (3.8 mL) was added ethane hydroxamic acid (0.1328 g, 1.769 mmol) and Potassium Carbonate (0.4074 g, 2.948 mmol). The reaction mixture was heated under microwave irradiation at 80° C. for 3 h. The reaction mixture was cooled to room temperature, acidified with 1 N hydrochloric acid and extracted with ethyl acetate (50 mL×4). The combined organic layer was dried over sodium sulphate and concentrated. The residue was purified by (200-400) silica using the ethyl acetate and cyclohexane (30:70), afforded white solid (400 mg, 61.80%).

¹H NMR (400 MHz, CHLOROFORM-d) δ ppm 7.48-7.65 (m, 2H) 6.80 (br d, 1H) 3.64-3.68 (m, 2H) 2.62 (tt, 1H) 2.27-2.46 (m, 2H) 1.97-2.12 (m, 2H) 1.29-1.37 (m, 2H) 1.07-1.22 (m, 2H)

### Step 3: Synthesis of 5-(5-chloropyrimidin-2-yl) oxy-2-cyclopropyl-4-(4,4,4-trifluorobutyl) quinoline-3-carbonitrile (1.044)

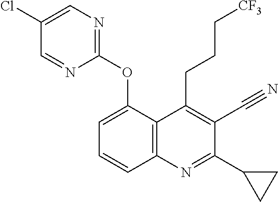 [SMILES: N#Cc1c(C2CC2)nc2cccc(Oc3ncc(Cl)cn3)c2c1CCCC(F)(F)F]

To a solution of 2-cyclopropyl-5-hydroxy-4-(4,4,4-trifluorobutyl) quinoline-3-carbonitrile (30 mg, 0.093 mmol), potassium carbonate (0.027 g, 0.28 mmol) and N, N-dimethylformamide (1.405 mL) was added 5-chloro-2-methylsulfonyl-pyrimidine (0.021 g, 0.11 mmol). The reaction mixture was stirred at 50° C. for 2 h, quenched with aqueous solution of ammonium chloride (20 mL), extracted in ethyl acetate (3×20 mL), dried over anhydrous sodium sulfate, filtered and concentrated under reduced pressure. The residue was purified by (200-400) silica using the ethyl acetate and cyclohexane (20:80), afforded yellow solid (14 mg, 34.54%).

¹H NMR (400 MHz, CHLOROFORM-d) δ ppm 8.57 (s, 2H) 7.91 (dd, 1H) 7.76 (t, 1H) 7.25 (dd, 1H) 3.43-3.51 (m, 2H) 2.60-2.68 (m, 1H) 2.17-2.34 (m, 2H) 1.85-1.95 (m, 2H) 1.34-1.39 (m, 2H) 1.17-1.24 (m, 2H)

### EXAMPLE 22: 4-BUT-3-ENYL-5-(5-CHLOROPYRIMIDIN-2-YL) OXY-2 (TRIFLUOROMETHYL) QUINAZOLINE (1.028)

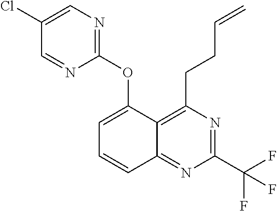 [SMILES: C=CCCc1nc(C(F)(F)F)nc2cccc(Oc3ncc(Cl)cn3)c12]

### Step 1: Synthesis of 4-but-3-enyl-5-methoxy-2-(trifluoromethyl)quinazoline

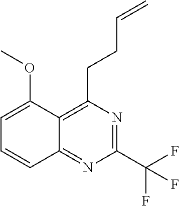 [SMILES: C=CCCc1nc(C(F)(F)F)nc2cccc(OC)c12]

To a solution of Lanthanum (III) trichloride bis (lithium chloride) complex (20 mL, 10.518 mmole) in tetrahydrofuran (20 mL) was added 5-methoxy-2-(trifluoromethyl) quinazoline (2 g, 8.76 mmol) in tetrahydrofuran (20 mL) under nitrogen at room temperature. This mixture was cooled to 0° C. and added bromo(but-3-enyl) magnesium (50 mL, 26.296 mmol) dropwise. This mixture was stirred at room temperature for overnight. The reaction was quenched with saturated solution of ammonium chloride (50 mL), extracted in ethyl acetate (3×50 mL), dried over anhydrous sodium sulfate, filtered and concentrated under reduced pressure to give 4-but-3-enyl-5-methoxy-2-(trifluoromethyl)-3,4-dihydroquinazoline (2.4 gm). The mixture (1.246 g, 3.944 mmol) was dissolved in anhydrous tetrahydrofuran (12 mL) then added 2,3-dichloro-5,6-dicyano-1,4-benzoquinone (2.74 g, 11.83 mmol) in one portion. The reaction mass was stirred for 2 h, then quenched with 2M solution of sodium hydroxide (20 mL), extracted in ethyl acetate (3×50 mL), dried over anhydrous sodium sulfate, filtered and concentrated under reduced pressure. The residue was purified by (200-400) silica using the ethyl acetate and cyclohexane (40:60), afforded (630 mg, 56.58%).

¹H NMR (400 MHz, CHLOROFORM-d) 7.88 (t, 1H) 7.75 (d, 1H) 7.09 (d, 1H) 5.85-6.12 (m, 1H) 5.09-5.16 (m, 1H) 5.04 (br d, 1H) 4.06 (s, 3H) 3.57-3.67 (m, 2H) 2.59 (br dd, 2H)

### Step 2: Synthesis of 4-but-3-enyl-2-(trifluoromethyl) quinazolin-5-ol

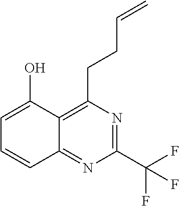 [SMILES: C=CCCc1nc(C(F)(F)F)nc2cccc(O)c12]

To a solution of 4-but-3-enyl-5-methoxy-2-(trifluoromethyl) quinazoline (230 mg, 0.81 mmol) in dichloromethane (2.3 mL) was added boron tribromide in dichloromethane (2.4 mL, 2.44 mmol) at 0° C. dropwise. The reaction mass was stirred at room temperature for 1 hour and quenched with aqueous solution of sodium bicarbonate solution (20 mL), extracted in ethyl acetate (3×20 mL), dried over anhydrous sodium sulfate, filtered and concentrated under reduced pressure. The residue was purified by (200-400) silica using the ethyl acetate and cyclohexane (40:60), afforded greenish black solid (179 mg, 81.92%).

¹H NMR (400 MHz, CHLOROFORM-d) δ ppm 7.68-7.80 (m, 2H) 7.10 (dd, 1H) 5.98 (d, 1H) 5.09 (dd, 1H) 4.99 (dd, 1H) 3.65-3.75 (m, 2H) 2.64 (br dd, 2H)

### Step 3: Synthesis of 4-but-3-enyl-5-(5-chloropyrimidin-2-yl) oxy-2 (trifluoromethyl)quinazoline (1.028)

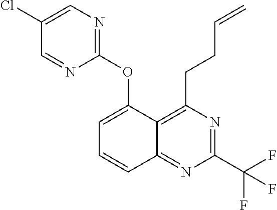 [SMILES: C=CCCc1nc(C(F)(F)F)nc2cccc(Oc3ncc(Cl)cn3)c12]

To a solution of 4-but-3-enyl-2-(trifluoromethyl) quinazolin-5-ol (288 mg, 1.074 mmol), in isopropyl alcohol (3 mL) was added 5-chloro-2-methylsulfonyl-pyrimidine (0.310 g, 1.611 mmol) and potassium carbonate (0.445 g, 3.221 mmol). The reaction mixture was then heated at 50° C. for 16 h, quenched with aqueous solution of ammonium chloride (20 mL), extracted in ethyl acetate (3×20 mL), dried over anhydrous sodium sulfate, filtered and concentrated under reduced pressure. The residue was purified by (200-400) silica using the ethyl acetate and cyclohexane (20:80), afforded yellow solid (150 mg, 36.68%).

¹H NMR (400 MHz, CHLOROFORM-d) δ ppm 8.53 (s, 2H), 8.14 (d, 1H), 7.99 (t, 1H), 7.48 (d, 1H), 5.86 (dd, 1H), 4.90-5.02 (m, 2H), 3.44-3.50 (m, 2H), 2.57 (td, 2H)

### EXAMPLE 23: 5-(5-CHLOROPYRIMIDIN-2-YL)OXY-2-ISOPROPYL-4-(4,4,4-TRIFLUOROBUTYL) QUINAZOLINE (1.050)

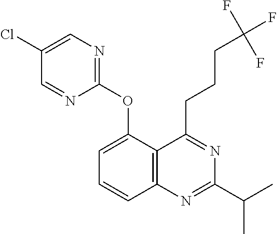 [SMILES: CC(C)c1nc(CCCC(F)(F)F)c2c(Oc3ncc(Cl)cn3)cccc2n1]

### Step 1: Synthesis of 5-methoxy-1H-quinazoline-2,4-dione

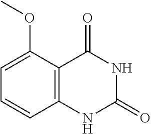

To a suspension of 2-amino-6-methoxy-benzoic acid (5.03 g, 30.1 mmol) in water (120 mL) and acetic acid (3.5 mL, 61 mmol) at 35° C. was added sodium cyanate (4.15 g, 61.3 mmol) and the resulting mixture was stirred for 0.5 hours. The mixture was treated with sodium hydroxide (50 mass % in water, 45 mL, 873 mmol) to give homogeneous solution which was warmed to 70° C. and stirred for 4 hours. On completion the mixture was cooled over ice, acidified with concentrated hydrochloric acid and the precipitate was collected and dried to give 5-methoxy-1H-quinazoline-2,4-dione (5.14 g, 84%) as an off-white solid. 1H NMR (400 MHz, DMSO-d6) 5=10.91 (br s, 2H), 7.51 (t, 1H), 6.72 (d, 1H), 6.70 (d, 1H), 3.81 (s, 3H)

### Step 2: Synthesis of 2,4-dichloro-5-methoxy-quinazoline

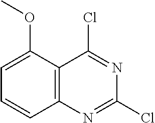

A suspension of 5-methoxy-1H-quinazoline-2,4-dione (5.136 g, 25.39 mmol) in acetonitrile (25 mL) and phosphoryl chloride (7.5 mL, 79 mmol) was stirred under an atmosphere of nitrogen at 90° C. for 17 hours. On completion, the mixture was concentrated and the residues subjected to flash column chromatography (ethyl acetate and cyclohexane) to give 2,4-dichloro-5-methoxy-quinazoline (3.45 g, 56%) as a white solid. 1H NMR (400 MHz, chloroform) 5=7.86 (t, 1H), 7.56 (dd, 1H), 7.03 (d, 1H), 4.04 (s, 3H)

### Step 3: Synthesis of 2-chloro-5-methoxy-4-(4,4,4-trifluorobutyl)quinazoline

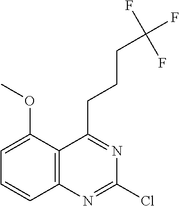 [SMILES: COc1cccc2nc(Cl)nc(CCCC(F)(F)F)c12]

To stirred magnesium turnings (250 mg, 10.29 mmol) in tetrahydrofuran (8.5 mL) was added a crystal of iodine. The reaction was heated to 50° C. and a small amount of 1-bromo-4,4,4-trifluorobutane in tetrahydrofuran was added. On activation the remaining 1-bromo-4,4,4-trifluorobutane (0.52 mL, 4.1 mmol) was added dropwise and the mixture was stirred for an hour at 50° C. The above solution was added dropwise to a solution 2,4-dichloro-5-methoxy-quinazoline (514 mg, 2.13 mmol) and copper(I) iodide (81 mg, 0.43 mmol) in tetrahydrofuran (8.5 mL) at 0° C. The mixture was stirred for 15 minutes, quenched with saturated aqueous ammonium chloride, extracted with ethyl acetate and concentrated. The residues were subjected to flash column chromatography (ethyl acetate and cyclohexane) to give 2-chloro-5-methoxy-4-(4,4,4-trifluorobutyl)quinazoline (514 mg, 71%) as an off-white solid.

1H NMR (400 MHz, chloroform) 5=7.80 (t, 1H), 7.55 (dd, 1H), 6.98 (d, 1H), 4.04 (s, 3H), 3.54-3.48 (m, 2H), 2.36-2.21 (m, 2H), 2.15-2.05 (m, 2H)

### Step 4: Synthesis of 2-isopropyl-5-methoxy-4-(4,4,4-trifluorobutyl)quinazoline

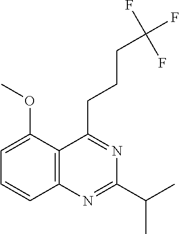 [SMILES: COc1cccc2nc(C(C)C)nc(CCCC(F)(F)F)c12]

A solution of 2-chloro-5-methoxy-4-(4,4,4-trifluorobutyl)quinazoline (149 mg, 0.46 mmol) and iron(III) acetylacetonate (19 mg, 0.05 mmol) in tetrahydrofuran (3.0 mL) and NMP (0.3 mL) was cooled over ice, treated with isopropylmagnesium chloride (2.0 M in THF, 0.70 mL, 1.4 mmol) and stirred for 5 minutes. The mixture was quenched with saturated aqueous ammonium chloride, extracted with ethyl acetate and concentrated. The residues were subjected to flash column chromatography (ethyl acetate and cyclohexane) to give 2-isopropyl-5-methoxy-4-(4,4,4-trifluorobutyl)quinazoline (97 mg, 64%) as a yellow oil. 1H NMR (400 MHz, chloroform) 5=7.70 (t, 1H), 7.55 (dd, 1H), 6.88 (d, 1H), 4.00 (s, 3H), 3.50 (t, 2H), 3.26 (spt, 1H), 2.36-2.21 (m, 2H), 2.18-2.08 (m, 2H), 1.40 (d, 6H)

### Step 5: Synthesis of 2-isopropyl-4-(4,4,4-trifluorobutyl)quinazolin-5-ol

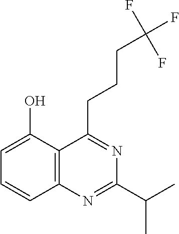 [SMILES: CC(C)c1nc(CCCC(F)(F)F)c2c(O)cccc2n1]

A solution of 2-isopropyl-5-methoxy-4-(4,4,4-trifluorobutyl)quinazoline (97 mg, 0.31 mmol) in NMP (0.8 mL) was treated with 1-dodecanethiol (190 μL, 0.78 mmol) and sodium hydroxide (50 mass % in water, 40 μL, 0.78 mmol) before being stirred at 100° C. for an hour. The mixture was quenched with saturated aqueous ammonium chloride, extracted with ethyl acetate and concentrated. The residues were subjected to flash column chromatography (ethyl acetate and cyclohexane) to give 2-isopropyl-4-(4,4,4-trifluorobutyl)quinazolin-5-ol (52 mg, 53%) as a beige solid. 1H NMR (400 MHz, chloroform) 5=7.57-7.46 (m, 2H), 6.92 (d, 1H), 3.71 (t, 2H), 3.35 (spt, 1H), 2.38-2.17 (m, 4H), 1.43 (d, 6H)

### Step 6: Synthesis of 5-(5-chloropyrimidin-2-yl)oxy-2-isopropyl-4-(4,4,4-trifluorobutyl)quinazoline (1.050)

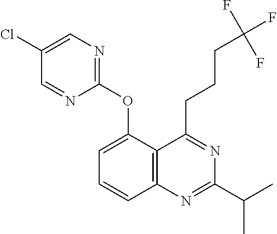 [SMILES: CC(C)c1nc(CCCC(F)(F)F)c2c(Oc3ncc(Cl)cn3)cccc2n1]

A mixture of 2-isopropyl-4-(4,4,4-trifluorobutyl)quinazolin-5-ol (52 mg, 0.17 mmol), 5-chloro-2-(methylsulfonyl)pyrimidine (106 mg, 0.55 mmol) and potassium carbonate (71 mg, 0.51 mmol) in propan-2-ol (1.0 mL) was stirred at 50° C. for 7 hours. On completion the mixture was diluted with water, extracted with ethyl acetate and concentrated. The residues were subjected to flash column chromatography (ethyl acetate and cyclohexane) to give 5-(5-chloropyrimidin-2-yl)oxy-2-isopropyl-4-(4,4,4-trifluorobutyl)quinazoline (51 mg, 71%) as a beige solid. 1H NMR (400 MHz, chloroform) 5=8.53 (s, 2H), 7.93 (d, 1H), 7.82 (t, 1H), 7.25 (d, 1H), 3.40-3.24 (m, 3H), 2.30-2.14 (m, 2H), 2.14-2.03 (m, 2H), 1.41 (d, 6H)

### EXAMPLE 24: 5-(5-CHLOROPYRIMIDIN-2-YL)OXY-2-METHOXY-4-(4,4,4-TRIFLUOROBUTYL) (1.014)

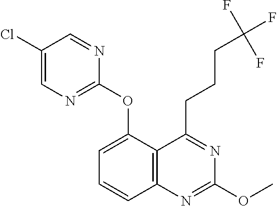 [SMILES: COc1nc(CCCC(F)(F)F)c2c(Oc3ncc(Cl)cn3)cccc2n1]

### Step 1: Synthesis of 2-bromo-4-(4,4,4-trifluorobutyl)quinazolin-5-ol

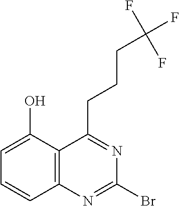 [SMILES: Oc1cccc2nc(Br)nc(CCCC(F)(F)F)c12]

A solution of 2-chloro-5-methoxy-4-(4,4,4-trifluorobutyl)quinazoline (1.03 g, 3.38 mmol) in chloroform (11 mL) was treated with boron tribromide (1.0 M in dichloromethane, 10 mL, 10 mmol) and stirred at 60° C. for 18 hours. The mixture was quenched with water, basified to pH=5 and extracted with ethyl acetate. The organics were concentrated and subjected to flash column chromatography (ethyl acetate and cyclohexane) to give 2-bromo-4-(4,4,4-trifluorobutyl)quinazolin-5-ol (889 mg, 71%) as an orange solid. 1H NMR (400 MHz, chloroform) 5=7.76-7.66 (m, 1H), 7.59-7.54 (m, 1H), 6.98-6.90 (m, 1H), 6.29 (s, 1H), 3.65-3.51 (m, 2H), 2.37-2.22 (m, 2H), 2.21-2.09 (m, 2H)

### Step 2: Synthesis of 2-[[2-bromo-4-(4,4,4-trifluorobutyl)quinazolin-5-yl]oxymethoxy]ethyl-trimethyl-silane

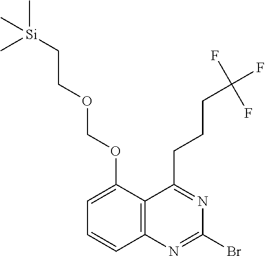 [SMILES: C[Si](C)(C)CCOCOc1cccc2nc(Br)nc(CCCC(F)(F)F)c12]

A mixture of 2-bromo-4-(4,4,4-trifluorobutyl)quinazolin-5-ol (889 mg, 2.3875 mmol), potassium carbonate (501 mg, 3.6249 mmol) and acetonitrile (6 mL) was treated with 2-(trimethylsilyl)ethoxymethyl chloride (550 μL, 2.95 mmol) and stirred at room temperature for an hour. The mixture was concentrated and subjected to flash column chromatography (ethyl acetate and cyclohexane) to give 2-[[2-bromo-4-(4,4,4-trifluorobutyl)quinazolin-5-yl]oxymethoxy]ethyl-trimethyl-silane (948 mg, 81%) as a yellow oil. 1H NMR (400 MHz, chloroform) 5=7.82-7.74 (m, 1H), 7.60 (dd, 1H), 7.33-7.28 (m, 1H), 5.45 (s, 2H), 3.86-3.77 (m, 2H), 3.56-3.47 (m, 2H), 2.38-2.22 (m, 2H), 2.19-2.07 (m, 2H), 1.05-0.95 (m, 2H), 0.00 (s, 9H)

### Step 3: Synthesis of 2-methoxy-4-(4,4,4-trifluorobutyl)quinazolin-5-ol

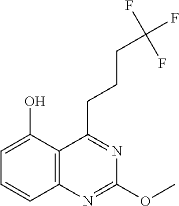 [SMILES: COc1nc(CCCC(F)(F)F)c2c(O)cccc2n1]

A solution of 2-[[2-bromo-4-(4,4,4-trifluorobutyl)quinazolin-5-yl]oxymethoxy]ethyl-trimethyl-silane (111 mg, 0.227 mmol) and sodium methoxide (0.5 M in MeOH, 1.0 mL, 0.5 mmol) in methanol (1.0 mL) was stirred at room temperature for 17 hours.

On completion the mixture was treated with 2 M hydrochloric acid (2 mL, 4.0 mmol) and was stirred at room temperature for 3 hours and then at 60° C. for a further 2.5 hours. The mixture was diluted with water, extracted with ethyl acetate and concentrated. The residues were subjected to flash column chromatography (ethyl acetate and cyclohexane) to give 2-methoxy-4-(4,4,4-trifluorobutyl)quinazolin-5-ol (40 mg, 59%) as a pale yellow solid. 1H NMR (400 MHz, chloroform) 5=7.60-7.53 (m, 1H), 7.4 (dd, 1H), 6.69 (dd, 1H), 5.75 (s, 1H), 4.10 (s, 3H), 3.51 (t, 2H), 2.33-2.20 (m, 2H), 2.20-2.09 (m, 2H)

### Step 4: Synthesis of 5-(5-chloropyrimidin-2-yl)oxy-2-methoxy-4-(4,4,4-trifluorobutyl)quinazoline (1.014)

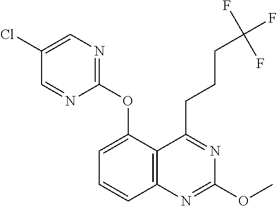 [SMILES: COc1nc(CCCC(F)(F)F)c2c(Oc3ncc(Cl)cn3)cccc2n1]

A mixture of 2-methoxy-4-(4,4,4-trifluorobutyl)quinazolin-5-ol (40 mg, 0.1328 mmol), 5-chloro-2-(methylsulfonyl)pyrimidine (79 mg, 0.41013 mmol) and potassium carbonate (56 mg, 0.40518 mmol) propan-2-ol (1.0 mL) was stirred at 50° C. for 5 hours. The mixture was diluted with water, extracted with ethyl acetate and concentrated. The residues were subjected to flash column chromatography (ethyl acetate and cyclohexane) to give 5-(5-chloropyrimidin-2-yl)oxy-2-methoxy-4-(4,4,4-trifluorobutyl)quinazoline (28 mg, 50%) as a beige solid.

TABLE 1
Examples of herbicidal compounds of the present invention.

| | |
|---|---|
| | (I) |
| 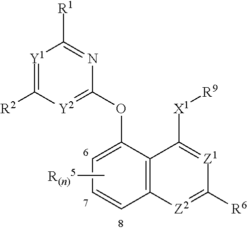 | |

| | | | | | | | | | | | 1H NMR (400 MHz, |
|---|---|---|---|---|---|---|---|---|---|---|---|
| Compound | R¹ | R² | Y¹ | Y² | R⁵ | R⁶ | X¹ | Z¹ | Z² | R⁹ | CDCl₃ unless stated) |
| 1.001 | H | H | C—Cl | N | — | H | O | CH | CH | 5- | 8.41 (s, 4H), 7.89 (dd, |
| | | | | | | | | | | chloropyrimidin- | 2H), 7.55 (t, 2H), 7.21 |
| | | | | | | | | | | 2-yl | (dd, 2H) |
| 1.002 | H | H | C—Cl | N | — | H | CH₂ | CH | CH | CH₂CH₂CF₃ | 8.50 (s, 2H), 7.85-7.78 |
| | | | | | | | | | | | (m, 2H), 7.48 (t, 1H), |
| | | | | | | | | | | | 7.40 (t, 1H), 7.30 (d, |
| | | | | | | | | | | | 1H), 7.20 (d, 1H), 3.10 |
| | | | | | | | | | | | (t, 2H), 2.20-2.07 (m, |
| | | | | | | | | | | | 2H), 1.92-1.83 (m, 2H) |
| 1.003 | H | H | C—Cl | N | — | H | CH₂ | N | N | CH₂CH₂CF₃ | 9.22 (s, 1H), 8.53 (s, |
| | | | | | | | | | | | 2H), 8.02 (dd, 1H), 7.90 |
| | | | | | | | | | | | (t, 1H), 7.37 (dd, 1H), |
| | | | | | | | | | | | 3.38 (t, 2H), 2.27-2.18 |
| | | | | | | | | | | | (m, 2H), 2.08-2.04 (m, |
| | | | | | | | | | | | 2H) |
| 1.004 | H | H | C—Cl | N | — | CF₃ | CH₂ | N | N | CH₂CH₂CF₃ | 8.54 (s, 2H), 8.16 (d, |
| | | | | | | | | | | | 1H), 8.02 (t, 1H), 7.51 |
| | | | | | | | | | | | (d, 1H), 3.47 (t, 2H), |
| | | | | | | | | | | | 2.28-2.21 (m, 2H), 2.12- |
| | | | | | | | | | | | 2.09 (m, 2H) |
| 1.005 | H | H | C—Cl | N | — | CF₃ | CH₂ | N | N | CH₂CH₂CH₃ | 8.53 (s, 2H), 8.14 (dd, |
| | | | | | | | | | | | 1H), 7.99 (t, 1H), 7.47 |
| | | | | | | | | | | | (dd, 1H), 3.35 (t, 2H), |
| | | | | | | | | | | | 1.76-1.70 (m, 2H), 1.41- |
| | | | | | | | | | | | 1.34 (m, 2H), 0.86 (t, |
| | | | | | | | | | | | 3H) |
| 1.006 | H | H | C—Cl | N | — | CF₃ | O | N | N | CH₂CH₂CF₃ | 8.47 (s, 2H), 8.08 (d, |
| | | | | | | | | | | | 1H), 8.00 (t, 1H), 7.48 |
| | | | | | | | | | | | (d, 1H), 4.67 (t, 2H), |
| | | | | | | | | | | | 2.48 (m, 2H) |
| 1.007 | H | H | C—Cl | N | — | CH₃ | O | N | N | CH₂CH₂CF₃ | 8.47 (s. 2H), 7.83 (m, |
| | | | | | | | | | | | 2H), 7.26 (m, 1H), 4.56 |
| | | | | | | | | | | | (t, 2H), 2.70 (s, 3H), 2.40 |
| | | | | | | | | | | | (m, 2H) |
| 1.008 | H | H | C—Cl | N | — | H | O | N | N | CH₂CH₂CF₃ | 8.79 (s, 1H), 8.47 (s, |
| | | | | | | | | | | | 2H), 7.95-7.88 (m, 2H), |
| | | | | | | | | | | | 7.35 (d, 1H), 4.58 (t, |
| | | | | | | | | | | | 2H), 2.49-2.35 (m, 2H) |
| 1.009 | H | H | C—Cl | N | — | H | O | N | CH | CH₂CH₂CF₃ | 8.45 (s, 2H), 7.98 (d, |
| | | | | | | | | | | | 1H), 7.70 (d, 2H), 7.33- |
| | | | | | | | | | | | 7.29 (m, 2H), 4.49 (t, |
| | | | | | | | | | | | 2H), 2.43-2.30 (m, 2H) |
| 1.010 | H | H | C—Cl | N | — | CH₃ | O | N | N | CH(CH₃)₂ | 8.45 (s, 2H), 7.79 (d, |
| | | | | | | | | | | | 2H), 7.23 (t, 1H), 5.52- |
| | | | | | | | | | | | 5.41 (m, 1H), 2.68 (s, |
| | | | | | | | | | | | 3H), 1.03 (d, 6H) |
| 1.011 | H | H | C—F | N | — | CF₃ | CH₂ | N | N | CH₂CH₂CF₃ | 8.47 (s, 2H), 8.14 (dd, |
| | | | | | | | | | | | 1H), 8.01 (t, 1H), 7.51 |
| | | | | | | | | | | | (dd, 1H), 3.48 (t, 2H), |
| | | | | | | | | | | | 2.27-2.21 (m, 2H), 2.12- |
| | | | | | | | | | | | 2.08 (m, 2H) |
| 1.012 | H | H | C—Cl | N | — | H | CH₂ | N | CH | CH₂CH₂CF₃ | 8.52 (s, 2H), 8.45 (d, 1H), |
| | | | | | | | | | | | 7.77 (dd, 1H), 7.68 (t, |
| | | | | | | | | | | | 1H), 7.58 (d, 1H), 7.30 |
| | | | | | | | | | | | (dd, 1H), 3.33-3.39 (m, |
| | | | | | | | | | | | 2H), 2.11-2.23 (m, 2H), |
| | | | | | | | | | | | 1.96-2.06 (m, 2H) |
| 1.013 | H | H | C—Cl | N | — | CH₃ | CH₂ | N | N | CH₂CH₂CF₃ | 8.53 (s, 2 H) 7.91 (dd, 1 |
| | | | | | | | | | | | H) 7.85 (t, 1 H) 7.29 (s, 1 |
| | | | | | | | | | | | H) 3.32 (t, 2 H) 2.85 (s, 3 |
| | | | | | | | | | | | H) 2.14-2.26 (m, 2 H) |
| | | | | | | | | | | | 1.97-2.07 (m, 2 H) |
| 1.014 | H | H | C—Cl | N | — | OCH₃ | CH₂ | N | N | CH₂CH₂CF₃ | 8.53 (s, 2H), |
| | | | | | | | | | | | 7.81-7.75 (m, 2H), |
| | | | | | | | | | | | 7.13 (dd, 1H), 4.13 (s, |
| | | | | | | | | | | | 3H), 3.31 (t, 2H), |
| | | | | | | | | | | | 2.28-2.12 (m, 2H), |
| | | | | | | | | | | | 2.10-2.00 (m, 2H) |
| 1.015 | H | H | C—Cl | N | — | CF₃ | CH₂ | N | N | CH₂CH₂CF₃ | 8.52 (s, 2 H), 8.21 (d, 1 |
| | | | | | | | | | | | H), 7.82 (t, 1 H), 7.53 (s, |
| | | | | | | | | | | | 1 H) 7.42 (dd, 1 H), 3.18- |
| | | | | | | | | | | | 3.25 (m, 2 H), 2.14- |
| | | | | | | | | | | | 2.27 (m, 2 H), 1.87- |
| | | | | | | | | | | | 1.98 (m, 2 H) |
| 1.016 | H | H | C—Cl | N | 7-CH₃ | CH₃ | CH₂ | N | N | CH₂CH₂CF₃ | 8.55 (s, 2H), 7.70 (s, 1H), |
| | | | | | | | | | | | 7.12 (s, 1H) 3.28 (t, 2H), |
| | | | | | | | | | | | 2.84 (s, 3H), 2.57 (s, 3H) |
| | | | | | | | | | | | 1.97-2.06 (m, 2H), |
| | | | | | | | | | | | 1.45 (s, 1H), 1.28 (s, 2H) |
| 1.017 | H | H | C—Cl | N | 8-CH₃ | CH₃ | CH₂ | N | N | CH₂CH₂CF₃ | 8.51 (s, 2 H) 7.68 (d, 1 |
| | | | | | | | | | | | H) 7.16 (d, 1 H) 3.29 (t, |
| | | | | | | | | | | | 2 H) 2.85 (s, 3 H) 2.75 |
| | | | | | | | | | | | (s, 3 H) 2.12-2.25 (m, 2 |
| | | | | | | | | | | | H) 1.95-2.04 (m, 2 H) |
| 1.018 | H | H | C—Cl | N | 7-CN | CH₃ | CH₂ | N | N | CH₂CH₂CF₃ | 8.55 (s, 2 H), 8.20 (d, 1 |
| | | | | | | | | | | | H), 7.47 (d, 1 H), 3.36 (t, |
| | | | | | | | | | | | 2 H), 2.86 (s, 3 H), 2.15- |
| | | | | | | | | | | | 2.27 (m, 2 H), 1.99- |
| | | | | | | | | | | | 2.08 (m, 2 H) |
| 1.019 | H | H | C—Cl | N | 8-Cl | CH₃ | CH₂ | N | N | CH₂CH₂CF₃ | 8.53 (s, 2 H) 7.96 (d, 1 |
| | | | | | | | | | | | H) 7.22-7.25 (d, 1 H) |
| | | | | | | | | | | | 3.33 (t, 2 H) 2.92 (s, 3 H) |
| | | | | | | | | | | | 2.14-2.26 (m, 2 H) 1.97- |
| | | | | | | | | | | | 2.08 (m, 2 H) |
| 1.020 | H | H | C—CH₃ | N | — | CF₃ | CH₂ | N | N | CH₂CH₂CF₃ | 8.49 (s, 2H), 8.11 (d, 1H), |
| | | | | | | | | | | | 7.99 (t, 1H), 7.50 (d, |
| | | | | | | | | | | | 1H), 3.48 (t, 2H), 2.31 (s, |
| | | | | | | | | | | | 3H), 2.29-2.13 (m, 2H), |
| | | | | | | | | | | | 2.11-2.01 (m, 2H) |
| 1.021 | H | H | C—Cl | N | — | CF₃ | CH₂ | N | N | CH₂CH₂CH₂CF₃ | 8.52 (s, 2H), 8.12 (d, |
| | | | | | | | | | | | 1H), 8.00 (t, 1H), 7.49 |
| | | | | | | | | | | | (d, 1H), 3.39 (t, 2H), |
| | | | | | | | | | | | 2.17-2.01 (m, 2H), 1.92- |
| | | | | | | | | | | | 1.82 (m, 2H), 1.68-1.58 |
| | | | | | | | | | | | (m, 2H) |
| 1.022 | H | H | C—Cl | N | — | CF₃ | CH₂ | N | N | CH₂CF₃ | 8.53 (s, 2H), 8.14 (d, |
| | | | | | | | | | | | 1H), 8.01 (t, 1H), 7.50 |
| | | | | | | | | | | | (d, 1H), 3.69-3.61 (m, |
| | | | | | | | | | | | 2H), 2.86-2.71 (m, 2H) |
| 1.023 | H | H | C—Cl | C—F | — | CF₃ | CH₂ | N | N | CH₂CH₂CF₃ | 8.09 (d, 1H), 7.98 (d, |
| | | | | | | | | | | | 1H), 7.97-7.91 (m, 1H), |
| | | | | | | | | | | | 7.67 (dd, 1H), 7.40 (dd, |
| | | | | | | | | | | | 1H), 3.52 (t, 2H), 2.31- |
| | | | | | | | | | | | 2.11 (m, 4H). |
| 1.024 | H | OCH₃ | N | C—H | — | CF₃ | CH₂ | N | N | CH₂CH₂CF₃ | 8.36 (s, 1H), 8.05 (d, |
| | | | | | | | | | | | 1H), 7.91 (t, 1H), 7.34 |
| | | | | | | | | | | | (d, 1H), 6.31 (s, 1H), |
| | | | | | | | | | | | 3.98 (s, 3H), 3.33 (t, 2H), |
| | | | | | | | | | | | 2.22-2.01 (m, 4H) |
| 1.025 | H | H | C—Br | N | — | CF₃ | CH₂ | N | N | CH₂CH₂CF₃ | 8.51 (s, 2H), 8.14 (d, |
| | | | | | | | | | | | 1H), 8.00 (t, 1H), 7.50 |
| | | | | | | | | | | | (d, 1H), 3.45 (t, 2H), |
| | | | | | | | | | | | 2.30-2.15 (m, 2H), 2.15- |
| | | | | | | | | | | | 2.03 (m, 2H) |
| 1.026 | H | H | C—OCH₃ | N | — | CF₃ | CH₂ | N | N | CH₂CH₂CF₃ | 8.26 (s, 2H), 8.10 (d, |
| | | | | | | | | | | | 1H), 7.98 (t, 1H), 7.46 |
| | | | | | | | | | | | (d, 1H), 3.91 (s, 3H), |
| | | | | | | | | | | | 3.51 (t, 2H), 2.30-2.14 |
| | | | | | | | | | | | (m, 2H), 2.13-2.02 (m, |
| | | | | | | | | | | | 2H) |
| 1.027 | H | H | C—OCHF₂ | N | — | CF₃ | CH₂ | N | N | CH₂CH₂CF₃ | 8.48 (s, 2H), 8.15 (d, |
| | | | | | | | | | | | 1H), 8.01 (t, 1H), 7.52 |
| | | | | | | | | | | | (d, 1H), 6.58 (t, 1H), |
| | | | | | | | | | | | 3.47 (t, 2H), 2.31-2.17 |
| | | | | | | | | | | | (m, 2H), 2.13-2.02 (m, |
| | | | | | | | | | | | 2H) |
| 1.028 | H | H | C—Cl | N | — | CF₃ | CH₂ | N | N | —CH₂CH═CH₂ | 8.53 (s, 2 H), 8.14 (d, 1 |
| | | | | | | | | | | | H), 7.99 (t, 1 H), 7.48 (d, |
| | | | | | | | | | | | 1 H), 5.86 (dd, 1 H), 4.90- |
| | | | | | | | | | | | 5.02 (m, 2 H), 3.44- |
| | | | | | | | | | | | 3.50 (m, 2 H), 2.57 (td, 2 |
| | | | | | | | | | | | H) |
| 1.029 | H | H | C—Cl | N | — | Br | CH₂ | N | N | CH₂CH₂CF₃ | 8.54 (s, 2H), 7.98-7.88 |
| | | | | | | | | | | | (m, 2H), 7.39 (dd, 1H), |
| | | | | | | | | | | | 3.40-3.32 (m, 2H), 2.29- |
| | | | | | | | | | | | 2.13 (m, 2H), 2.09-1.98 |
| | | | | | | | | | | | (m, 2H) |
| 1.030 | H | H | C—Cl | N | — | OCH₂CF₃ | CH₂ | N | N | CH₂CH₂CF₃ | 8.53 (s, 2H), 7.86-7.81 |
| | | | | | | | | | | | (m, 1H), 7.79 (dd, 1H), |
| | | | | | | | | | | | 7.21 (dd, 1H), 4.95 (q, |
| | | | | | | | | | | | 2H), 3.35 (t, 2H), 2.27- |
| | | | | | | | | | | | 2.12 (m, 2H), 2.12-1.98 |
| | | | | | | | | | | | (m, 2H) |
| 1.031 | H | H | C—Cl | N | — | c-pentyl | CH₂ | N | N | CH₂CH₂CF₃ | 8.52 (s, 2H), 7.91 (dd, |
| | | | | | | | | | | | 1H), 7.82 (t, 1H), 7.24 |
| | | | | | | | | | | | (dd, 1H), 3.45 (quin, 1H), |
| | | | | | | | | | | | 3.33 (t, 2H), 2.27-1.96 |
| | | | | | | | | | | | (m, 8H), 1.94-1.82 (m, |
| | | | | | | | | | | | 2H), 1.79-1.66 (m, 2H) |
| 1.032 | H | H | C—Cl | N | — | n-hexyl | CH₂ | N | N | CH₂CH₂CF₃ | 8.53 (s, 2H), 7.92 (dd, |
| | | | | | | | | | | | 1H), 7.83 (t, 1H), 7.26 |
| | | | | | | | | | | | (dd, 1H), 3.33 (t, 2H), |
| | | | | | | | | | | | 3.07-3.02 (m, 2H), 2.28- |
| | | | | | | | | | | | 2.13 (m, 2H), 2.10-1.98 |
| | | | | | | | | | | | (m, 2H), 1.94-1.83 (m, |
| | | | | | | | | | | | 2H), 1.48-1.26 (m, 6H), |
| | | | | | | | | | | | 0.92-0.85 (m, 3H) |
| 1.033 | H | H | C—Cl | N | — | n-propyl | CH₂ | N | N | CH₂CH₂CF₃ | 8.53 (s, 2H), 7.92 (dd, |
| | | | | | | | | | | | 1H), 7.83 (t, 1H), 7.27 |
| | | | | | | | | | | | (dd, 1H), 3.33 (t, 2H), |
| | | | | | | | | | | | 3.06-3.00 (m, 2H), 2.27- |
| | | | | | | | | | | | 2.13 (m, 2H), 2.10-1.99 |
| | | | | | | | | | | | (m, 2H), 1.98-1.87 (m, |
| | | | | | | | | | | | 2H), 1.04 (t, 3H) |
| 1.034 | H | H | C—Cl | N | 7-Br | CH₃ | CH₂ | N | N | CH₂CH₂CF₃ | 8.55 (s, 2 H), 8.08 (d, 1 |
| | | | | | | | | | | | H), 7.41 (d, 1 H) 3.29 |
| | | | | | | | | | | | (t, , 2H) 2.83 (s, |
| | | | | | | | | | | | 3 H) 2.13-2.25 (m, 2 H) |
| | | | | | | | | | | | 1.96-2.05 (m, 2 H) |
| 1.035 | H | H | C—Cl | N | — | CH₃ | CH₂ | C | N | CH₂CH₂CF₃ | 8.50 (s, 2 H), 7.99 (d, 1 |
| | | | | | | | | | | | H), 7.67 (t, 1 H), 7.21 |
| | | | | | | | | | | | (d, 1 H), 7.08 (s, 1 H), |
| | | | | | | | | | | | 3.02-3.11 (m, 2 H), |
| | | | | | | | | | | | 2.70 (s, 3 H), 2.08-2.22 |
| | | | | | | | | | | | (m, 2 H), 1.81-1.96 (m, |
| | | | | | | | | | | | 2 H) |
| 1.036 | H | H | C—Cl | N | — | c-propyl | CH₂ | N | N | CH₂CH₂CF₃ | 8.52 (s, 2H), 7.86 (dd, |
| | | | | | | | | | | | 1H), 7.79 (t, 1H), 7.19 |
| | | | | | | | | | | | (dd, 1H), 3.28 (t, 2H), |
| | | | | | | | | | | | 2.37-2.29 (m, 1H), 2.24- |
| | | | | | | | | | | | 2.10 (m, 2H), 2.08-1.97 |
| | | | | | | | | | | | (m, 2H), 1.29-1.22 (m, |
| | | | | | | | | | | | 2H), 1.15-1.08 (m, 2H) |
| 1.037 | H | H | C—Cl | N | — | H | CH₂ | N | N | CH₂CH₂CF₃ | 9.20 (s, 1H), 7.94 (m, |
| | | | | | | | | | | | 2H), 7.85 (t, 1H), 7.65 |
| | | | | | | | | | | | (d, 1H), 7.28 (m, 1H), |
| | | | | | | | | | | | 3.45 (t, 2H), 2.10-2.28 |
| | | | | | | | | | | | (m, 4H) |
| 1.038 | H | H | C—OCHF₂ | N | — | H | CH₂ | N | N | CH₂CH₂CF₃ | 9.21 (s, 1H), 8.48 (s, |
| | | | | | | | | | | | 2H), 8.02 (d, 1H), 7.91 |
| | | | | | | | | | | | (t, 1H), 7.40 (d, 1H), |
| | | | | | | | | | | | 6.58 (t, 1H), 3.42 (t, 2H), |
| | | | | | | | | | | | 2.24 (m, 2H), 2.08 (m, |
| | | | | | | | | | | | 2H) |
| 1.039 | H | H | C—CN | N | — | H | CH₂ | N | N | CH₂CH₂CF₃ | 9.24 (s, 1H), 8.87 (s, |
| | | | | | | | | | | | 2H), 8.08 (d, 1H), 7.94 |
| | | | | | | | | | | | (t, 1H), 7.40 (d, 1H), |
| | | | | | | | | | | | 3.34 (t, 2H), 2.24 (m, |
| | | | | | | | | | | | 2H), 2.08 (m, 2H) |
| 1.040 | H | H | C—Cl | N | — | CH₃ | CH₂ | C—Cl | N | CH₂CH₂CF₃ | 8.51 (s, 2 H), 7.97 (dd, 1 |
| | | | | | | | | | | | H), 7.67 (dd, 1 H), 7.21- |
| | | | | | | | | | | | 7.25 (m, 1 H), 3.32- |
| | | | | | | | | | | | 3.44 (m, 2 H), 2.82 (s, 3 |
| | | | | | | | | | | | H), 2.12-2.29 (m, 2 H), |
| | | | | | | | | | | | 1.70-1.83 (m, 2 H) |
| 1.041 | H | H | C—Cl | N | — | CH₃ | CH₂ | C—Br | N | CH₂CH₂CF₃ | 8.51 (s, 2 H), 7.98 (d, 1 |
| | | | | | | | | | | | H), 7.69 (t, 1 H), 7.24 (d, |
| | | | | | | | | | | | 1 H), 3.36-3.49 (m, 2 |
| | | | | | | | | | | | H), 2.89 (s, 3 H), 2.12- |
| | | | | | | | | | | | 2.33 (m, 2 H), 1.74-1.82 |
| | | | | | | | | | | | (m, 2 H) |
| 1.042 | H | H | C—Cl | N | — | CH₃ | CH₂ | C—CH₃ | N | CH₂CH₂CF₃ | 8.52 (s, 2 H) 7.99 (br d, |
| | | | | | | | | | | | 1 H) 7.62 (t, 1 H) 7.14- |
| | | | | | | | | | | | 7.22 (m, 1 H) 3.14-3.26 |
| | | | | | | | | | | | (m, 2 H) 2.73 (s, 3 H) |
| | | | | | | | | | | | 2.42 (s, 3 H) 2.15-2.29 |
| | | | | | | | | | | | (m, 2 H) 1.69-1.83 (m, |
| | | | | | | | | | | | 2 H) |
| 1.043 | H | H | C—Cl | N | — | CH₃ | CH₂ | C—CN | N | CH₂CH₂CF₃ | 8.53 (s, 2 H), 8.01 (d, 1 |
| | | | | | | | | | | | H), 7.82 (t, 1 H), 7.33 (d, |
| | | | | | | | | | | | 1 H), 3.38-3.53 (m, 2 |
| | | | | | | | | | | | H), 2.91 (s, 3 H), 2.19- |
| | | | | | | | | | | | 2.33 (m, 2 H), 1.83- |
| | | | | | | | | | | | 1.93 (m, 2 H) |
| 1.044 | H | H | C—Cl | N | — | C₃H₅ | CH₂ | C—CN | N | CH₂CH₂CF₃ | 8.46-8.57 (m, 2 H) 7.76 |
| | | | | | | | | | | | (t, 1 H) 7.91 (dd, 1 H) |
| | | | | | | | | | | | 7.25 (dd, 1 H) 3.43-3.51 |
| | | | | | | | | | | | (m, 2 H) 2.60-2.68 (m, |
| | | | | | | | | | | | 1 H) 2.17-2.34 (m, 2 H) |
| | | | | | | | | | | | 1.85-1.95 (m, 2 H) |
| | | | | | | | | | | | 1.34-1.39 (m, 2 H) |
| | | | | | | | | | | | 1.17-1.24 (m, 2 H) |
| 1.045 | H | H | C—Cl | N | — | CF₃ | CH₂ | C—CN | N | CH₂CH₂CF₃ | 8.55 (s, 2 H), 8.24 (dd, |
| | | | | | | | | | | | 1 H), 7.99 (t, 1 H), 7.57 |
| | | | | | | | | | | | (dd, 1 H), 3.56-3.64 |
| | | | | | | | | | | | (m, 2 H), 2.24-2.38 (m, |
| | | | | | | | | | | | 2 H), 1.87-1.97 (m, 2 |
| | | | | | | | | | | | H) |
| 1.046 | H | H | C—Cl | N | 8-CH₃ | CF₃ | CH₂ | N | N | CH₂CH₂CF₃ | 8.53 (s, 2 H) 7.86 (dd, 1 |
| | | | | | | | | | | | H) 7.40 (d, 1 H) 3.44 (t, |
| | | | | | | | | | | | 2 H) 2.83 (s, 3 H) 2.17- |
| | | | | | | | | | | | 2.30 (m, 2 H) 2.05-2.14 |
| | | | | | | | | | | | (m, 2 H) |
| 1.047 | H | H | C—Cl | N | 8-CN | CF₃ | CH₂ | N | N | CH₂CH₂CF₃ | 8.59 (s, 2 H) 8.39 (d, 1 |
| | | | | | | | | | | | H) 7.61 (d, 1 H) 3.55 (t, |
| | | | | | | | | | | | 2 H) 2.20- |
| | | | | | | | | | | | 2.32 (m, 2 H) 2.08-2.20 |
| | | | | | | | | | | | (m, 2 H) |
| 1.048 | H | H | C—Cl | N | 8-Br | CF₃ | CH₂ | N | N | CH₂CH₂CF₃ | 8.54 (s, 2 H) 8.32 (m, 1 |
| | | | | | | | | | | | H) 7.41 (m, 1 H) 3.48 (t, |
| | | | | | | | | | | | 2 H) 2.04-2.28 (m, 4 H) |
| 1.049 | H | H | C—CN | N | — | CF₃ | CH₂ | N | N | CH₂CH₂CF₃ | 8.79 (s, 2H), 8.19 (d, |
| | | | | | | | | | | | 1H), 7.97 (t, 1H), 7.47 |
| | | | | | | | | | | | (d, 1H), 3.32 (t, 2H), |
| | | | | | | | | | | | 2.25-2.09 (m, 2H), 2.08- |
| | | | | | | | | | | | 1.98 (m, 2H) |
| 1.050 | H | H | C—Cl | N | — | C₃H₇ | CH₂ | N | N | CH₂CH₂CF₃ | 8.53 (s, 2H), 7.93 (d, |
| | | | | | | | | | | | 1H), 7.82 (t, 1H), 7.25 |
| | | | | | | | | | | | (d, 1H), 3.40-3.24 (m, |
| | | | | | | | | | | | 3H), 2.30-2.14 (m, 2H), |
| | | | | | | | | | | | 2.14-2.03 (m, 2H), 1.41 |
| | | | | | | | | | | | (d, 6H) |
| 1.051 | H | OCH₃ | N | C—H | — | H | CH₂ | N | N | CH₂CH₂CF₃ | 9.20 (s, 1H), 8.45 (s, |
| | | | | | | | | | | | 1H), 8.00 (d, 1H), 7.90 |
| | | | | | | | | | | | (t, 1H), 7.30 (d, 1H), |
| | | | | | | | | | | | 6.32 (s, 1H), 4.04 (s, |
| | | | | | | | | | | | 3H), 3.35 (t, 2H), 2.20 |
| | | | | | | | | | | | (m, 2H), 2.10 (m, 2H) |

### Biological Examples

Seeds of a variety of test species are sown in standard soil in pots Amaranthus retoflexus (AMARE), Echinochloa crus-galli (ECHCG), Setaria faberi (SETFA)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| Compound | AMARE | ECHCG | SETFA |
|---|---|---|---|
| 1.001 | 3 | 4 | 4 |
| 1.002 | 3 | 2 | 3 |
| 1.003 | 5 | 3 | 3 |
| 1.005 | 5 | 5 | 5 |
| 1.006 | 5 | 3 | 3 |
| 1.007 | 3 | 1 | 1 |
| 1.008 | 3 | 1 | 1 |
| 1.009 | 1 | 1 | 1 |
| 1.011 | 5 | 5 | 5 |
| 1.012 | 4 | 4 | 4 |
| 1.013 | 3 | 4 | 4 |
| 1.014 | 5 | 5 | 5 |
| 1.015 | 5 | 4 | 4 |
| 1.019 | 4 | 3 | 2 |
| 1.020 | 4 | 5 | 5 |
| 1.021 | 5 | 4 | 4 |
| 1.022 | 4 | 4 | 4 |
| 1.025 | 4 | 5 | 4 |
| 1.026 | 4 | 3 | 4 |
| 1.027 | 4 | 1 | 1 |
| 1.028 | 5 | 4 | 4 |
| 1.029 | 4 | 1 | 2 |
| 1.031 | 4 | 3 | 3 |
| 1.032 | 5 | 3 | 3 |
| 1.033 | 4 | 3 | 3 |
| 1.036 | 4 | 3 | 2 |
| 1.037 | 5 | 3 | 3 |
| 1.043 | 2 | 1 | 2 |
| 1.046 | 4 | 3 | 2 |
| 1.050 | 5 | 2 | 3 |

TABLE B2
Pre-emergence Test

| Compound | AMARE | ECHCG | SETFA |
|---|---|---|---|
| 1.001 | 3 | 3 | 4 |
| 1.002 | 1 | 1 | 1 |
| 1.003 | 5 | 5 | 5 |
| 1.005 | 5 | 5 | 5 |
| 1.006 | 5 | 3 | 5 |
| 1.007 | 2 | 1 | 3 |
| 1.008 | 5 | 1 | 5 |
| 1.009 | 1 | 1 | 1 |
| 1.011 | 5 | 5 | 5 |
| 1.012 | 4 | 5 | 5 |
| 1.013 | 3 | 4 | 5 |
| 1.014 | 5 | 5 | 5 |
| 1.015 | 5 | 5 | 5 |
| 1.019 | 2 | 4 | 4 |
| 1.020 | 5 | 5 | 5 |
| 1.021 | 5 | 5 | 5 |
| 1.022 | 5 | 5 | 5 |
| 1.025 | 4 | 5 | 5 |
| 1.026 | 2 | 4 | 4 |
| 1.027 | 3 | 2 | 0 |
| 1.028 | 4 | 5 | 5 |
| 1.029 | 1 | 1 | 4 |
| 1.031 | 1 | 5 | 5 |
| 1.032 | 4 | 4 | 4 |
| 1.033 | 2 | 5 | 5 |
| 1.036 | 2 | 4 | 5 |
| 1.037 | 2 | 2 | 4 |
| 1.043 | 0 | 1 | 2 |
| 1.046 | 2 | 2 | 3 |
| 1.050 | 5 | 5 | 5 |

1. A compound of Formula (I): 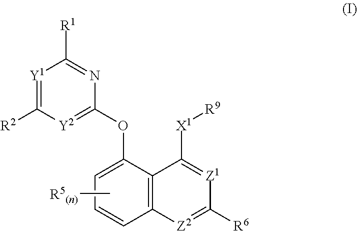
or an agronomically acceptable salt thereof,
wherein
Y¹ is N or CR³;
Y² is N or CR⁴;
with the proviso that Y¹ and Y² are not both N;
R¹ is selected from the group consisting of hydrogen, halogen, C₁-C₃alkyl and C₁-C₃haloalkyl;
R² is selected from the group consisting of hydrogen, halogen, C₁-C₃alkyl, C₁-C₃alkoxy-, C₁-C₃haloalkoxy- and C₁-C₃haloalkyl;
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
each R⁵ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁶ is independently selected from the group consisting of hydrogen, hydroxy, C₁-C₆alkyl, C₁-C₆haloalkyl-, C₁-C₆alkoxy-, C₁-C₆haloalkoxy- and C₃-C₆cycloalkyl-;
X¹ is CH₂ or O;
Z¹ is N or CR⁷;
Z² is N or CR⁸;
R⁷ is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁸ is is selected from the group consisting of hydrogen, C₁-C₄alkyl, halogen, —CN, nitro, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R⁹ is selected from the group consisting of C₁-C₆alkyl, C₁-C₆haloalkyl- and pyrimidin-2-yl wherein the pyrimidin-2-yl is optionally substituted by 1 or 2 substituents independently selected from the group consisting of halogen, CN, C₁-C₂ alkyl, C₁-C₂ alkoxy- and C₁-C₂ haloalkoxy-;
n=0, 1 or 2; and
p=0, 1 or 2.

2. A compound according to claim 1, wherein R¹ and R² are both hydrogen.

3. A compound according to claim 1, wherein Y¹ is CR³ and Y² is N.

4. A compound according to claim 3, wherein R³ is chloro.

5. A compound according to claim 1, wherein n=0.

6. A compound according to claim 1, wherein R⁶ is selected from the group consisting of hydrogen, methyl and CF₃.

7. A compound according to claim 1, wherein Z¹ is CH and Z² is CH.

8. A compound according to claim 1, wherein Z¹ is N and Z² is N.

9. A compound according to claim 1, wherein X¹ is O.

10. A compound according to claim 1, wherein R⁹ is C₁-C₆alkyl or C₁-C₆haloalkyl-.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as a herbicide.
